(19) United States
(12) Patent Application Publication
(10) Pub. No.: US 2021/0341371 A1
(43) Pub. Date: Nov. 4, 2021

(54) CELL SCANNING TECHNOLOGIES AND METHODS OF USE THEREOF

(71) Applicants: Thomas Adam Shine, Brookline, MA (US); Ian Basil Shine, Swampscott, MA (US)

(72) Inventors: Thomas Adam Shine, Brookline, MA (US); Ian Basil Shine, Swampscott, MA (US)

(21) Appl. No.: 17/311,089

(22) PCT Filed: Dec. 4, 2019
(86) PCT No.: PCT/US19/64543

§ 371 Date: Jun. 4, 2021

Related U.S. Application Data
(60) Provisional application No. 62/775,703, filed on Dec. 5, 2018.

(51) Int. Cl. G01N 15/08; G01N 15/06; G01N 33/50

(52) U.S. Cl. CPC G01N 15/08; G01N 2015/149; G01N 33/5094; G01N 15/06

(57) ABSTRACT

Diagnostic and screening technologies, therapy recommendations, and computer systems based on red blood cell membrane permeability characteristics are provided herein.

### CROSS REFERENCE TO RELATED APPLICATIONS

The present application claims priority to U.S. Provisional Patent Application No. 62/775,703, filed Dec. 5, 2018, the entire contents of which are hereby incorporated by reference.

### BACKGROUND

Early diagnosis of disease has numerous benefits for patient care and treatment outcomes. For example, an extensive worldwide analysis of cancer survival rates concluded that survival trends are attributable to differences in early diagnosis and the corresponding available treatments. See Allemani, C. et al., The Lancet 385(9972), 977-1010.

### SUMMARY

The present disclosure provides technologies for screening and/or diagnosing subjects. Among other things, the present disclosure provides the recognition that certain cell characteristics (e.g., red blood cell (RBC) characteristics), and in particular certain RBC membrane permeability characteristics, can reveal important feature(s) relevant to health of human subjects. The present disclosure demonstrates that certain cell membrane permeability parameters (e.g., RBC membrane permeability parameters) provided herein are useful for detecting and/or diagnosing many different diseases, disorders, and conditions. The present disclosure also provides the recognition that changes in an individual's RBC membrane permeability characteristics over time are useful for monitoring health and/or response to administered therapy.

Provided technologies can be used for identifying and/or characterizing subjects in need of diagnostic assessment or therapeutic intervention (e.g., by determining one or more RBC permeability parameters and comparing them to a reference control parameter). In some embodiments, the present disclosure provides technologies for monitoring a subject over time, e.g., while receiving therapy, and optionally initiating, terminating, or adjusting therapy based on monitoring results.

Provided technologies can be used for identifying and/or characterizing agents as RBC Permeability Modulating Agents (e.g., by contacting a sample of RBCs with an agent, determining one or more RBC permeability parameters and comparing them to a reference control parameter).

Also provided herein are technologies for monitoring viability of blood (e.g., donated blood).

### BRIEF DESCRIPTION OF THE DRAWINGS

FIG. 1, comprising panels a-f, shows an exemplary cell permeability analysis of a healthy individual. FIG. 1a is a graph of data collected in a cell-by-cell analysis showing the voltage recorded for individual red blood cells of a healthy individual over decreasing osmolality (in a range from 280 mOsm/kg to 54 mOsm/kg). Population density is represented by color, with zero density corresponding to white, the lowest nonzero density corresponding to the darkest points (e.g., blue), and, as density progressively increases, color of the points lightens (e.g., from green to yellow to orange to red to black to aqua). FIG. 1b is a graph of change in cell volume with respect to change in osmolality of a test sample (“Cell Scan Plot”). FIG. 1c is a fluid flux curve (FFC) plotting the percent change of rate of fluid flux with respect to changes in osmolality of a test sample. FIG. 1d is a frequency distribution graph of three “cuts” of the cell-by-cell curve of FIG. 1a. The “cuts” correspond to three osmolality ranges: the solid thin line 107 being isotonic (resting) cells (i.e., 280 mOsm/kg), bold line 109 being spherical cells (i.e., 142 mOsm/kg), and dotted line 108 being ghost cells (i.e., 110 mOsm/kg). FIG. 1e is an illustrative embodiment of the cell size and shape at the isotonic osmolality. FIG. if shows superimposed graphs of mean voltage 111 and cell count 110 for the test against osmolality.

FIG. 2, comprising panels a-d, shows varying degrees of severity of cell fragmentation. FIG. 2a is an example of a cell-by-cell graph with a low degree of cell fragmentation. FIG. 2b is an example of a cell-by-cell graph with a moderate degree of cell fragmentation. FIG. 2c is an example of a cell-by-cell graph with a severe degree of cell fragmentation. FIG. 2d is an example of a cell-by-cell graph with a very severe degree of cell fragmentation.

FIG. 3, comprising panels a-c, shows exemplary methods for determining scattering of a RBC permeability analysis (e.g., heterogeneity of the cell population). Scattering (i.e., cell diversity or cell scattering) can be determined, e.g., from a cell-by-cell graph (FIG. 3a), from a frequency distribution curve (FIG. 3b), and/or from a fluid flux curve (FIG. 3c).

FIG. 4A, comprising panels a-f, shows an exemplary cell permeability analysis of an unhealthy individual suffering from cancer of unknown primary origin. FIG. 4A-a is a graph of data collected in a cell-by-cell analysis showing the voltage recorded for individual red blood cells of the unhealthy individual over decreasing osmolality (in a range from 280 mOsm/kg to 54 mOsm/kg). Population density is represented by color, with zero density corresponding to white, the lowest nonzero density corresponding to the darkest points (e.g., blue), and, as density progressively increases, color of the points lightens (e.g., from green to yellow to orange to red to black to aqua). FIG. 4A-b is a graph of percentage volume change of red blood cells with respect to changes in osmolality of a test sample (“Cell Scan Plot”). FIG. 4A-c is a fluid flux curve (FFC) plotting the percent change of rate of fluid flux with respect to changes in osmolality of a test sample. FIG. 4A-d is a frequency distribution graph of three “cuts” of the cell-by-cell curve of FIG. 4A-a. The “cuts” correspond to three osmolality ranges: the solid thin line 107 being isotonic (resting) cells (i.e., approx. 280 mOsm/kg), bold line 109 being spherical cells (i.e., approx. 142 mOsm/kg), and bold line 108 being ghost cells (i.e., approx. 110 mOsm/kg). FIG. 4A-e is an illustrative embodiment of the cell size and shape at the isotonic osmolality. FIG. 4A-f shows superimposed graphs of mean voltage 111 and cell count 110 for the test, respectively, against osmolality.

FIG. 4B, comprising panels a-f, shows an exemplary cell permeability analysis of an unhealthy individual suffering from cirrhosis. FIG. 4B-a is a graph of data collected in a cell-by-cell analysis showing the voltage recorded for individual red blood cells of the unhealthy individual over decreasing osmolality (in a range from 280 mOsm/kg to 54 mOsm/kg). Population density is represented by color, with zero density corresponding to white, the lowest nonzero density corresponding to the darkest points (e.g., blue), and, as density progressively increases, color of the points lightens (e.g., from green to yellow to orange to red to black to aqua). FIG. 4B-b is a graph of percentage volume change of red blood cells with respect to changes in osmolality of a test sample (“Cell Scan Plot”). FIG. 4B-c is a fluid flux curve (FFC) plotting the percent change of rate of fluid flux with respect to changes in osmolality of a test sample. FIG. 4B-d is a frequency distribution graph of three “cuts” of the cell-by-cell curve of FIG. 4B-a. The “cuts” correspond to three osmolality ranges: the solid thin line 107 being isotonic (resting) cells (i.e., approx. 280 mOsm/kg), bold line 109 being spherical cells (i.e., approx. 142 mOsm/kg), and dotted line 108 being ghost cells (i.e., approx. 110 mOsm/kg). FIG. 4B-e is an illustrative embodiment of the cell size and shape at the isotonic osmolality. FIG. 4B-f shows superimposed graphs of mean voltage 111 and cell count 110 for the test, respectively, against osmolality.

FIG. 4C, comprising panels a-f, shows an exemplary cell permeability analysis of an unhealthy individual suffering from malignancy of unknown origin. FIG. 4C-a is a graph of data collected in a cell-by-cell analysis showing the voltage recorded for individual red blood cells of the unhealthy individual over decreasing osmolality (in a range from 280 mOsm/kg to 54 mOsm/kg). Population density is represented by color, with zero density corresponding to white, the lowest nonzero density corresponding to the darkest points (e.g., blue), and, as density progressively increases, color of the points lightens (e.g., from green to yellow to orange to red to black to aqua). FIG. 4C-b is a graph of percentage volume change of red blood cells with respect to changes in osmolality of a test sample (“Cell Scan Plot”). FIG. 4C-c is a fluid flux curve (FFC) plotting the percent change of rate of fluid flux with respect to changes in osmolality of a test sample. FIG. 4C-d is a frequency distribution graph of three “cuts” of the cell-by-cell curve of FIG. 4C-a. The “cuts” correspond to three osmolality ranges: the solid thin line 107 being isotonic (resting) cells (i.e., approx. 280 mOsm/kg), bold line 109 being spherical cells (i.e., approx. 142 mOsm/kg), and dotted line 108 being ghost cells (i.e., approx. 110 mOsm/kg). FIG. 4C-e is an illustrative embodiment of the cell size and shape at the isotonic osmolality. FIG. 4C-f shows superimposed graphs of mean voltage 111 and cell count 110 for the test, respectively, against osmolality.

FIG. 5 shows exemplary Cell Scan shapes characteristic of particular diseases, disorders, and conditions. Cell Scan shapes are labeled as follows: normal (N); leukemia/lymphoma (L); pancreatic/lung cancer (P); gastrointestinal tract malignancies (G); preleukemic myelodysplasia (MF); beta thalassemia heterozygotes/hemoglobin S homozygotes/hemoglobin C homozygotes (T); hereditary spherocytosis/hemolytic anemias (HS); liver disease/cirrhosis (C).

FIG. 6, comprising panels A-E, shows exemplary Fluid Flux Curve (FFC) shapes characteristics of particular diseases, disorders, and conditions obtained by overlaying patient scans. FIG. 6A is FFC Shape N, characteristic of normal (healthy) subjects. FIG. 6B is FFC Shape L, characteristic of subjects suffering from leukemia/lymphoma. FIG. 6C is FFC Shape P, characteristic of subjects suffering from pancreatic/lung cancer. FIG. 6D is FFC Shape G, characteristic of subjects suffering from gastrointestinal tract malignancies. FIG. 6E is FFC Shape T, characteristic of subjects suffering from beta thalassemia heterozygotes/hemoglobin S homozygotes/hemoglobin C homozygotes.

FIG. 7A shows a graph plotting number of months patients survived after Cell Scan vs. Pk0 of subjects for whom a date of death was confirmed (N=1586). Each data point in FIG. 7A represents mean duration of life for patients with that Pk0 value.

FIG. 7B shows a graph plotting number of months patients survived after Cell Scan vs. Pk0 of subjects for whom a date of death was confirmed and who were pregnant at the time of the Cell Scan. Each data point in FIG. 7B represents mean duration of life for patients with that Pk0 value.

FIG. 7C shows a graph plotting the number of months patients survived after Cell Scan vs. ∂ dynes of subjects for whom a date of death was confirmed and for whom all fourteen parameters were recorded (N=922). Each data point in FIG. 7C represents mean duration of life for patients with that 0 dynes value.

FIG. 8 shows a graph of number of viable units of stored blood over time.

FIG. 9A shows a Cell Scan Plot of a blood sample before and after exposure to HgCl₂ solution. FIG. 9B shows a Fluid Flux Curve of a blood sample after exposure to HgCl₂ solution.

FIG. 10 shows schematically an instrument used to sample and test blood cells.

FIG. 11 shows velocity profiles for the discharge of fluids from fluid delivery syringes of a gradient generator section of the instrument of FIG. 10.

FIG. 12 shows a block diagram illustrating the data processing steps used in the instrument of FIG. 10.

FIG. 13 shows an example of a three-dimensional plot of osmolality against measured voltage for cells of a blood sample analyzed in accordance with the WO 97/24598 disclosure.

FIG. 14 shows another example of a three-dimensional plot of osmolality against measured voltage which illustrates the frequency distribution of blood cells at intervals.

FIG. 15 shows a series of three-dimensional plots for a sample tested at hourly intervals.

FIG. 16 shows superimposed plots of osmolality (x-axis) against measured voltage and true volume, respectively.

FIGS. 17A-17D show the results for a blood sample. FIG. 17A shows a three-dimensional plot of measured voltage against osmolality. FIG. 17B shows a graph of osmolality against percentage change in measured voltage for a series of tests of a sample. FIG. 17C shows the results in a tabulated form. FIG. 17D shows superimposed graphs of mean voltage and cell count for the test, respectively, against osmolality.

FIG. 18 shows Price-Jones (frequency distribution) curves of the results shown in FIGS. 17A-17D.

FIG. 19 shows a graph of osmolality against cell volume and indicates a number of different measures of cell permeability.

FIG. 20 shows a graph of osmolality against net fluid flow.

### DETAILED DESCRIPTION

### Definitions

The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

As used herein, the term “administration” typically refers to the administration of a composition to a subject or system. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e.g. intrahepatic), mucosal, nasal, oral, rectal, subcutaneous, sublingual, topical, tracheal (e.g., by intratracheal instillation), vaginal, vitreal, etc. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

In general, the term “agent”, as used herein, may be used to refer to a compound or entity of any chemical class including, for example, a polypeptide, nucleic acid, saccharide, lipid, small molecule, metal, or combination or complex thereof. In appropriate circumstances, as will be clear from context to those skilled in the art, the term may be utilized to refer to an entity that is or comprises a cell or organism, or a fraction, extract, or component thereof. Alternatively or additionally, as context will make clear, the term may be used to refer to a natural product in that it is found in and/or is obtained from nature. In some instances, again as will be clear from context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents may be provided as collections or libraries, for example that may be screened to identify or characterize active agents within them. In some cases, the term “agent” may refer to a compound or entity that is or comprises a polymer; in some cases, the term may refer to a compound or entity that comprises one or more polymeric moieties. In some embodiments, the term “agent” may refer to a compound or entity that is not a polymer and/or is substantially free of any polymer and/or of one or more particular polymeric moieties. In some embodiments, the term may refer to a compound or entity that lacks or is substantially free of any polymeric moiety.

As used herein “cell membrane permeability” refers to a property of a cell or population of cells (e.g., RBCs) that describes the ability of one or more molecule(s) or entities to pass through the cell membrane. In some embodiments, cell membrane permeability may be quantified or characterized by reference to Pk0. Alternatively or additionally, in some embodiments, cell membrane permeability may be quantified or characterized by reference to one or more of a cell-by-cell color map, fluid flux curve, Pymax, and/or Pymin. Still further alternatively or additionally, in some embodiments, cell membrane permeability may be quantified or characterized using technology such as that described herein, in, e.g., Example 1, and/or in the Prior Shine Technologies. Cells with lesser cell membrane permeability may be described as “resistant” or in a “resistant state,” i.e., the cells are more resistant to the intake of the one or more molecule(s) or entities, such as water. In many embodiments described herein, a relevant cell membrane permeability is that of cell membrane permeability to water.

As used herein, the term “comparable” refers to two or more agents, entities, situations, sets of conditions, circumstances, individuals, or populations, etc., that may not be identical to one another but that are sufficiently similar to permit comparison there between so that one skilled in the art will appreciate that conclusions may reasonably be drawn based on differences or similarities observed. In some embodiments, comparable agents, entities, situations, sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, circumstances, individuals, or populations, etc. to be considered comparable. For example, those of ordinary skill in the art will appreciate that sets of circumstances, agents, entities, situations, individuals, or populations are comparable to one another when characterized by a sufficient number and type of substantially identical features to warrant a reasonable conclusion that differences in results obtained or phenomena observed under or with different agents, entities, situations sets of circumstances, individuals, or populations are caused by or indicative of the variation in those features that are varied.

As used herein, the term “reference” describes a standard or control relative to which a comparison is performed. For example, in some embodiments, an agent, individual, population, sample, sequence or value of interest is compared with a reference or control agent, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

As used herein, the term “subject” refers an organism, typically a mammal (e.g., a human). In some embodiments, a subject is suffering from a relevant disease, disorder or condition. In some embodiments, a human subject is an adult, adolescent, or pediatric subject. In some embodiments, a subject is at risk of (e.g., susceptible to), e.g., at elevated risk of relative to an appropriate control individual or population thereof, a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is an individual to whom diagnosis and/or therapy and/or prophylaxis is and/or has been administered. The terms “subject” and “patient” are used interchangeably herein.

### Cell Scanning Technologies

The present disclosure encompasses the recognition that cell (e.g., RBC) membrane permeability is an important indicator of an individual's health. The present disclosure further appreciates that a convenient and accurate method of analyzing RBC membrane permeability is desirable for assessing the status of an individual's health. The present disclosure also encompasses the recognition that the provided technologies are particularly applicable to cells without a nucleus (e.g., making provided technologies universally applicable to a variety of organisms). In some embodiments, technologies for assessing membrane permeability are provided herein.

In some embodiments, the present disclosure describes application of and/or utilizes existing membrane permeability assessment technologies in a new context and use (e.g., with respect to particular individuals and/or populations), and documents that such application can achieve remarkable and unexpected results, particularly including diagnosis and/or determination of malarial susceptibility state for such individual(s) and/or population(s). In some embodiments, RBC membrane permeability can be measured using the devices and/or methods described in WO 97/24598, WO 97/24529, WO 97/24599, WO 97/24600, WO 97/24601, WO 00/39559, and WO 00/39560 (“Prior Shine Technologies”), each of which is hereby incorporated by reference in its entirety. Certain aspects of WO 97/24598 and WO 97/24601 are reproduced in Appendices A and B, respectively, and are contemplated in some embodiments of the present disclosure, both singly and in combination.

Alternatively or additionally, in some embodiments, the present disclosure describes and/or utilizes newly developed and/or improved membrane permeability assessment technologies, for example as described herein and/or in copending application titled “DEVICE” and filed by the same inventors on the same day as the instant application. In some embodiments, cell scanning technologies comprise mechanical pumps and/or fluid delivery systems (e.g., high resolution syringe pumps and syringes) that allow for achievement and/or maintenance of a desired cell concentration of a sample being passed to a sensor of an apparatus as the environment (e.g., pH, osmolality, agent concentration) of the sample is changed. In some embodiments, a uniform cell concentration within a tested sample passed to a sensor of a device is achieved by making an initial, standard fixed dilution of a biological sample with a diluent, counting a number of cells within a portion of the diluted sample by flowing the diluted sample and a diluent to a sensor (e.g., using computer-controlled, digital syringe pumps), and then adjusting the dilution ratio between the diluent and biological sample to achieve a desired cell concentration. In some embodiments, a concentration of cells in a biological sample is adjusted to a desired value by altering relative flow rates of biological sample and at least two other streams of liquid (e.g., one or more diluents), e.g., using a computer-controlled digital syringe. In some embodiments, cell scanning technologies comprise methods and apparatus to improve the throughput of samples by, for example, multiplexing the preparation and measurements of said samples. In some embodiments, cell scanning technologies comprise delivery of arbitrary gradients of one or more agents to a sensor of a device while maintaining a desired cell concentration of said sample being flowed to the sensor (e.g., using computer-controlled digital syringes). In some embodiments, cell scanning technologies comprise methods and apparatus for calibrating an apparatus, e.g., using one or more markers (e.g., fluorescent markers) or nanoparticles (e.g., latex beads), or e.g., using a sample (e.g., blood) from a healthy subject or population thereof (e.g., from one or more subjects previously determined and/or otherwise known not to be suffering from a condition or otherwise in a state that is associated with an “abnormal” reading as described herein). In some embodiments, cell scanning technologies comprise certain improvements and/or strategies that can achieve reduction(s) in mechanical and/or electrical noise, for example that might otherwise be transmitted through gradient generating systems (e.g., through an osmotic gradient generating system). In some embodiments, cell scanning technologies comprise technologies that can reduce and/or dampen one or more effects of mechanical noise, for example through incorporation of flexible tubing elements into the fluid flow path. In some embodiments, cell scanning technologies comprise systems in which a sensor is mechanically isolated. In some embodiments, cell scanning technologies comprise systems that include one or more electrically conducting components arranged and constructed, and/or otherwise associated with other components of the system, so that electrical noise experienced by the system is reduced and/or one or more components is shielded and/or grounded. In some embodiments, cell scanning technologies comprise two or more similar sample syringes are present and connected in parallel to one another at a substantially similar location in the fluid delivery path, e.g., in order to minimize refill and/or wash time of sample syringes between samples being tested. In some embodiments, cell scanning technologies comprise removing a blockage by temporarily reversing pressure within a sensor and/or expelling fluid from a syringe creating a reversal of fluid flow through the sensor. In some embodiments, a pressure across a sensor is constant and/or very well regulated (e.g., using digitally controlled syringes). In some embodiments, cell scanning technologies comprise methods and apparatus to allow for even mixing of a diluent and samples containing cells (e.g., by mixing at one or multiple locations within a fluid path).

In some embodiments, samples for use in cell scanning technologies described herein can be prepared according to standard procedures. Alternatively or additionally, in some embodiments, samples are prepared and/or analyzed as described in copending application titled “DEVICE” and filed by the same inventors on the same day as the instant application, for example ensuring uniform cell density and/or assessment of a plurality of dilutions of an obtained sample (e.g., a primary blood sample)

In some embodiments, a sample is a blood sample. In some embodiments, additional components (e.g., preservatives and/or anticoagulants) can be added to a blood sample. Additional components can include, but are not limited to, heparin, ACD, EDTA, and sodium citrate. Addition of typical preservatives and/or anticoagulants are not expected to significantly affect the output of cell scanning technologies provided herein. In some embodiments, if samples are compared, the samples are prepared and/or stored under comparable conditions.

In some embodiments, a blood sample may be a primary blood sample. In some embodiments, a blood sample may have been processed through one or more purification and/or separation steps. Alternatively or additionally, in some embodiments, a blood sample may have been processed through one or more dilution steps.

In some embodiments, a blood sample can be stored for a period of time prior to testing without significantly affecting the output of the cell scanning technologies provided herein. For example, a blood sample can be stored for up to about 1 hour, about 2 hours, about 3 hours, about 4 hours, about 5 hours, about 6 hours, about 12 hours, about 24 hours, about 48 hours, about 1 week, about 2 weeks, about 1 month, about 2 months, about 6 months, about 1 year, about 2 years, about 3 years, or longer without significantly affecting the output of the cell scanning technologies provided herein. In some embodiments, a blood sample can be stored at a particular temperature prior to testing without significantly affecting the output of the cell scanning technologies provided herein. For example, in some embodiments, a blood sample can be stored at about −80° C., about −20° C., about 0° C., about 10° C., about 20° C., or about 30° C. without significantly affecting the output of the cell scanning technologies provided herein.

### RBC Membrane Permeability Parameters

The present disclosure provides certain RBC membrane permeability parameters, obtainable using cell scanning technologies described herein, that are useful in provided methods (e.g., screening, diagnosing, and monitoring subjects, etc.).

In some embodiments, a RBC membrane permeability parameter is coefficient of permeability (Cp or Cpₙₑₜ). Cp represents the volume of water that passes through the cell membrane per unit area at maximum pressure. Cp can be calculated as described herein, e.g., in Appendix A. In some embodiments, a Cp of from about 2.7 mL/m² to about 5.1 mL/m², from about 3.1 mL/m² to about 4.7 mL/m², or from about 3.5 mL/m² to about 4.3 mL/m² is considered normal. In some embodiments, a Cp of about 3.1 mL/m², about 3.3 mL/m², about 3.5 mL/m², about 3.7 mL/m², about 3.9 mL/m², about 4.0 mL/m², about 4.1 mL/m², or about 4.3 mL/m² is considered normal. In some embodiments, a Cp of less than about 3.5 mL/m², about 3.1 mL/m², or about 2.7 mL/m², or greater than about 4.3 mL/m², about 4.7 mL/m², or about 5.1 mL/m² is considered abnormal. In some embodiments, a Cp of from about 0 mL/m² to about 2.7 mL/m², from about 0 mL/m² to about 3.1 mL/m², from about 0 mL/m² to about 3.5 mL/m², from about 4.3 mL/m² to about 10 mL/m², from about 4.7 mL/m² to about 10 mL/m², or from about 5.1 mL/m² to about 10 mL/m² is considered abnormal.

In some embodiments, a RBC membrane permeability parameter is Pk0. Pk0 represents the osmotic pressure at which a cell reaches maximum volume (e.g., before bursting). Pk0 can be calculated as described herein, e.g., in Appendix A, and/or from the peak of the Cell Scan Plot, e.g., as described in Example 1. In some embodiments, a Pk0 from about, 126.4 mOsm/kg to about 161.8 mOsm/kg, from about 132.3 mOsm/kg to about 155.9 mOsm/kg, or from about 138.2 mOsm/kg to about 150 mOsm/kg is considered normal. In some embodiments, a Pk0 of about 132 mOsm/kg, about 138 mOsm/kg, about 144 mOsm/kg, about 150 mOsm/kg, or about 156 mOsm/kg is considered normal. In some embodiments, a Pk0 of less than about 138 mOsm/kg, about 132 mOsm/kg, or about 126 mOsm/kg, or greater than about 150 mOsm/kg, about 150 mOsm/kg, or about 162 mOsm/kg is considered abnormal. In some embodiments, a Pk0 of from about 70 mOsm/kg to about 126 mOsm/kg, from about 70 mOsm/kg to about 132 mOsm/kg, from about 70 mOsm/kg to about 138 mOsm/kg, from about 150 mOsm/kg to about 275 mOsm/kg, from about 156 mOsm/kg to about 275 mOsm/kg, or from about 162 mOsm/kg to about 275 mOsm/kg is considered abnormal. In some embodiments, a Pk0 of from about 132 mOsm/kg to about 164 mOsm/kg, from about 137 mOsm/kg to about 159 mOsm/kg, or from about 142 mOsm/kg to about 153 mOsm/kg is considered normal. In some embodiments, a Pk0 of about 137 mOsm/kg, about 142 mOsm/kg, about 148 mOsm/kg, about 153 mOsm/kg, or about 159 mOsm/kg is considered normal. In some embodiments, a Pk0 of less than about 142 mOsm/kg, about 137 mOsm/kg, or about 132 mOsm/kg, or greater than about 153 mOsm/kg, about 159 mOsm/kg, or about 164 mOsm/kg is considered abnormal. In some embodiments, a Pk0 of from about 50 mOsm/kg to about 132 mOsm/kg, from about 50 mOsm/kg to about 137 mOsm/kg, from about 50 mOsm/kg to about 142 mOsm/kg, from about 153 mOsm/kg to about 290 mOsm/kg, from about 159 mOsm/kg to about 290 mOsm/kg, or from about 164 mOsm/kg to about 290 mOsm/kg is considered abnormal.

In some embodiments, a RBC membrane permeability parameter is isotonic volume (IsoV or Volumeᵢₛₒ). IsoV represents cell volume under isotonic conditions. IsoV can be determined as described herein, e.g., in Appendix A. In some embodiments, an IsoV of from about 77 fL to about 106 fL, from about 82 fL to about 101 fL, or from about 87 fL to about 96 fL is considered normal. In some embodiments, an IsoV of about 82 fL, about 87 fL, about 92 fL, about 96 fL, or about 101 fL is considered normal. In some embodiments, an IsoV of less than about 87 fL, about 82 fL, or about 77 fL, or greater than about 96 fL, about 101 fL, or about 106 fL is considered abnormal. In some embodiments, an IsoV of from about 50 fL to about 77 fL, from about 50 fL to about 82 fL, from about 50 fL to about 87 fL, from about 96 fL to about 150 fL, from about 101 fL to about 150 fL, or from about 106 fL to about 150 fL is considered abnormal.

In some embodiments, a RBC membrane permeability parameter is spherical volume (SphV or Volumeₛₚₕ). SphV represents maximum cell volume (i.e., spherical volume). In some embodiments, SphV is calibrated against spherical latex particles. SphV can be determined as described herein, e.g., in Appendix A. In some embodiments, a SphV of from about 136 fL to about 202 fL, from about 147 fL to about 191 fL, or from about 158 fL to about 180 fL is considered normal. In some embodiments, a SphV of about 147 fL, about 158 fL, about 169 fL, about 180 fL, or about 191 fL is considered normal. In some embodiments, a SphV of less than about 158 fL, about 147 fL, or about 136 fL, or greater than about 180 fL, about 191 fL, or about 202 fL is considered abnormal. In some embodiments, a SphV of from about 90 fL to about 136 fL, from about 90 fL to about 147 fL, from about 90 fL to about 158 fL, from about 180 fL to about 280 fL, from about 191 fL to about 280 fL, or from about 202 fL to about 280 fL is considered abnormal. In some embodiments, a SphV of from about 126 fL to about 201 fL, from about 138 fL to about 189 fL, or from about 151 fL to about 176 fL is considered normal. In some embodiments, a SphV of about 138 fL, about 151 fL, about 164 fL, about 176 fL, or about 189 fL is considered normal. In some embodiments, a SphV of less than about 151 fL, about 138 fL, or about 126 fL, or greater than about 176 fL, about 189 fL, or about 201 fL is considered abnormal. In some embodiments, a SphV of from about 90 fL to about 126 fL, from about 90 fL to about 138 fL, from about 90 fL to about 151 fL, from about 176 fL to about 280 fL, from about 189 fL to about 280 fL, or from about 201 fL to about 280 fL is considered abnormal.

In some embodiments, a RBC membrane permeability parameter is maximum % change in volume (Inc %). Inc % represents maximum % change in cell volume, i.e., the % change at Pk0. Inc % can be determined as described herein, e.g., from the Cell Scan Plot of Example 1. In some embodiments, an Inc % of from about 61% to about 108%, from about 69% to about 100%, or from about 77% to about 93% is considered normal. In some embodiments, an Inc % of about 69%, about 77%, about 85%, about 93%, or about 100% is considered normal. In some embodiments, an Inc % of less than about 61%, about 69%, or about 77%, or greater than about 93%, about 100%, or about 108% is considered abnormal. In some embodiments, an Inc % of from about 0% to about 61%, from about 0% to about 69%, from about 0% to about 77%, from about 93% to about 200%, from about 100% to about 200%, or from about 108% to about 200% is considered abnormal.

In some embodiments, a RBC membrane permeability parameter is peak width of Cell Scan Plot at 10% below maximum height (W10). W10 is indicative of cell homogeneity and cell diversity and can be determined from the Cell Scan Plot of Example 1. In some embodiments, a W10 of from about 15 mOsm/kg to about 22 mOsm/kg, from about 16 mOsm/kg to about 21 mOsm/kg, or from about 17 mOsm/kg to about 20 mOsm/kg is considered normal. In some embodiments, a W10 of about 16 mOsm/kg, about 17 mOsm/kg, about 18 mOsm/kg, about 19 mOsm/kg, about 20 mOsm/kg, or about 21 mOsm/kg is considered normal. In some embodiments, a W10 of less than about 15 mOsm/kg, about 16 mOsm/kg, or about 17 mOsm/kg, or greater than about 20 mOsm/kg, about 21 mOsm/kg, or about 22 mOsm/kg is considered abnormal. In some embodiments, a W10 of from about 5 mOsm/kg to about 15 mOsm/kg, from about 5 mOsm/kg to about 16 mOsm/kg, from about 5 mOsm/kg to about 17 mOsm/kg, from about 20 mOsm/kg to about 50 mOsm/kg, from about 21 mOsm/kg to about 50 mOsm/kg, or from about 22 mOsm/kg to about 50 mOsm/kg is considered abnormal. In some embodiments, a W10 of from about 13 mOsm/kg to about 21 mOsm/kg, from about 15 mOsm/kg to about 20 mOsm/kg, or from about 16 mOsm/kg to about 20 mOsm/kg is considered normal. In some embodiments, a W10 of about 15 mOsm/kg, about 16 mOsm/kg, about 17 mOsm/kg, about 18 mOsm/kg, about 19 mOsm/kg, or about 20 mOsm/kg is considered normal. In some embodiments, a W10 of less than about 13 mOsm/kg, about 15 mOsm/kg, or about 16 mOsm/kg, or greater than about 19 mOsm/kg, about 20 mOsm/kg, or about 21 mOsm/kg is considered abnormal. In some embodiments, a W10 of from about 5 mOsm/kg to about 13 mOsm/kg, from about 5 mOsm/kg to about 15 mOsm/kg, from about 5 mOsm/kg to about 16 mOsm/kg, from about 19 mOsm/kg to about 50 mOsm/kg, from about 20 mOsm/kg to about 50 mOsm/kg, or from about 21 mOsm/kg to about 50 mOsm/kg is considered abnormal.

In some embodiments, a RBC membrane permeability parameter is Pxmax (i.e., Cpmax). Pxmax is the osmolality at which the Fluid Flux Curve (e.g., of Example 1) is at maximum % fluid flux. In some embodiments, a Pxmax of from about 149 mOsm/kg to about 180 mOsm/kg, from about 154 mOsm/kg to about 175 mOsm/kg, or from about 159 mOsm/kg to about 170 mOsm/kg is considered normal. In some embodiments, a Pxmax of about 154 mOsm/kg, about 159 mOsm/kg, about 165 mOsm/kg, about 170 mOsm/kg, or about 175 mOsm/kg is considered normal. In some embodiments, a Pxmax of less than about 159 mOsm/kg, about 154 mOsm/kg, or about 149 mOsm/kg, or greater than about 170 mOsm/kg, about 175 mOsm/kg, or about 180 mOsm/kg is considered abnormal. In some embodiments, a Pxmax of from about 50 mOsm/kg to about 149 mOsm/kg, from about 50 mOsm/kg to about 154 mOsm/kg, from about 50 mOsm/kg to about 159 mOsm/kg, from about 170 mOsm/kg to about 290 mOsm/kg, from about 175 mOsm/kg to about 290 mOsm/kg, or from about 180 mOsm/kg to about 290 mOsm/kg is considered abnormal.

In some embodiments, a RBC membrane permeability parameter is Pxmin (i.e., Cpmin). Pxmin is the osmolality at which the Fluid Flux Curve (e.g., of Example 1) is at minimum % fluid flux. In some embodiments, a Pxmin of from about 111 mOsm/kg to about 149 mOsm/kg, from about 118 mOsm/kg to about 143 mOsm/kg, or from about 124 mOsm/kg to about 137 mOsm/kg is considered normal. In some embodiments, a Pxmin of about 118 mOsm/kg, about 124 mOsm/kg, about 130 mOsm/kg, about 137 mOsm/kg, or about 143 mOsm/kg is considered normal. In some embodiments, a Pxmin of less than about 124 mOsm/kg, about 118 mOsm/kg, or about 111 mOsm/kg, or greater than about 137 mOsm/kg, about 143 mOsm/kg, or about 149 mOsm/kg is considered abnormal. In some embodiments, a Pxmin of from about 50 mOsm/kg to about 111 mOsm/kg, from about 50 mOsm/kg to about 118 mOsm/kg, from about 50 mOsm/kg to about 124 mOsm/kg, from about 137 mOsm/kg to about 290 mOsm/kg, from about 143 mOsm/kg to about 290 mOsm/kg, or from about 149 mOsm/kg to about 290 mOsm/kg is considered abnormal.

In some embodiments, a RBC membrane permeability parameter is Pymax. Pymax is the maximum fluid flux on the Fluid Flux Curve (e.g., of Example 1). In some embodiments, a Pymax of from about 9 (fL·10⁻¹)/mOsm/kg to about 16 (fL·10⁻¹)/mOsm/kg, from about 10 (fL·10⁻¹)/mOsm/kg to about 15 (fL·10⁻¹)/mOsm/kg, or from about 12 (fL·10⁻¹)/mOsm/kg to about 14 (fL·10⁻¹)/mOsm/kg is considered normal. In some embodiments, a Pymax of about 10 (fL·10⁻¹)/mOsm/kg, about 12 (fL·10⁻¹)/mOsm/kg, about 13 (fL·10⁻¹)/mOsm/kg, about 14 (fL·10⁻¹)/mOsm/kg, or about 15 (fL·10⁻¹)/mOsm/kg is considered normal. In some embodiments, a Pymax of less than about 12 (fL·10⁻¹)/mOsm/kg, about 10 (fL·10⁻¹)/mOsm/kg, or about 9 (fL·10⁻¹)/mOsm/kg, or greater than about 14 (fL·10⁻¹)/mOsm/kg, about 15 (fL·10⁻¹)/mOsm/kg, or about 16 (fL·10⁻¹)/mOsm/kg is considered abnormal. In some embodiments, a Pymax of from about 1 (fL·10⁻¹)/mOsm/kg to about 9 (fL·10⁻¹)/mOsm/kg, from about 1 (fL·10⁻¹)/mOsm/kg to about 10 (fL·10⁻¹)/mOsm/kg, from about 1 (fL·10⁻¹)/mOsm/kg to about 12 (fL·10⁻¹)/mOsm/kg, from about 14 (fL·10⁻¹)/mOsm/kg to about 50 (fL·10⁻¹)/mOsm/kg, from about 15 (fL·10⁻¹)/mOsm/kg to about 50 (fL·10⁻¹)/mOsm/kg, or about 16 (fL·10⁻¹)/mOsm/kg to about 50 (fL·10⁻¹)/mOsm/kg is considered abnormal.

In some embodiments, a RBC membrane permeability parameter is Pymin. Pymin is the minimum fluid flux on the Fluid Flux Curve (e.g., of Example 1). In some embodiments, a Pymin of from about −11 (fL·10⁻¹)/mOsm/kg to about −28 (fL·10⁻¹)/mOsm/kg, from about −14 (fL·10⁻¹)/mOsm/kg to about −25 (fL·10⁻¹)/mOsm/kg, or from about −17 (fL·10⁻¹)/mOsm/kg to about −22 (fL·10⁻¹)/mOsm/kg is considered normal. In some embodiments, a Pymin of about −14 (fL·10⁻¹)/mOsm/kg, about −17 (fL·10⁻¹)/mOsm/kg, about −20 (fL·10⁻¹)/mOsm/kg, about −22 (fL·10⁻¹)/mOsm/kg, or about −25 (fL·10⁻¹)/mOsm/kg is considered normal. In some embodiments, a Pymin of less than about −17 (fL·10⁻¹)/mOsm/kg, about −14 (fL·10⁻¹)/mOsm/kg, or about −11 (fL·10⁻¹)/mOsm/kg, or greater than about −22 (fL·10⁻¹)/mOsm/kg, about −25 (fL·10⁻¹)/mOsm/kg, or about −28 (fL·10⁻¹)/mOsm/kg is considered abnormal. In some embodiments, a Pymin of from about −1 (fL·10⁻¹)/mOsm/kg to about −11 (fL·10⁻¹)/mOsm/kg, from about −1 (fL·10⁻¹)/mOsm/kg to about −14 (fL·10⁻¹)/mOsm/kg, from about −1 (fL·10⁻¹)/mOsm/kg to about −17 (fL·10⁻¹)/mOsm/kg, from about −22 (fL·10⁻¹)/mOsm/kg to about −50 (fL·10⁻¹)/mOsm/kg, from about −25 (fL·10⁻¹)/mOsm/kg to about −50 (fL·10⁻¹)/mOsm/kg, or about −28 (fL·10⁻¹)/mOsm/kg to about −50 (fL·10⁻¹)/mOsm/kg is considered abnormal.

In some embodiments, a RBC membrane permeability parameter is Py ratio. Py ratio is the ratio of Pymax:Pymin in absolute values. In some embodiments, a Py ratio of from about 0.4 to about 1.0, from about 0.5 to about 0.9, or from about 0.6 to about 0.8 is considered normal. In some embodiments, a Py ratio of about 0.5, about 0.6, about 0.7, about 0.8, or about 0.9 is considered normal. In some embodiments, a Py ratio of less than about 0.4, about 0.5, or about 0.6, or greater than about 0.8, about 0.9, or about 1.0 is considered abnormal. In some embodiments, a Py ratio of from about 0.01 to about 0.4, from about 0.01 to about 0.5, from about 0.01 to about 0.6, from about 0.8 to about 10, from about 0.9 to about 10, or from about 1.0 to about 10 is considered abnormal.

In some embodiments, a RBC membrane permeability parameter is sphericity index (SI). Sphericity index can be determined as described herein, e.g., in Appendix A. In some embodiments, a sphericity index of from about 1.42 to about 1.72, from about 1.47 to about 1.67, or from about 1.52 to about 1.62 is considered normal. In some embodiments, a sphericity index of about 1.47, about 1.52, about 1.57, about 1.62, or about 1.67 is considered normal. In some embodiments, a sphericity index of less than about 1.42, about 1.47, or about 1.52, or greater than about 1.62, about 1.67, or about 1.72 is considered abnormal. In some embodiments, a sphericity index of from about 1.0 to about 1.42, from about 1.0 to about 1.47, from about 1.0 to about 1.52, from about 1.62 to about 3.0, from about 1.67 to about 3.0, or from about 1.72 to about 3.0 is considered abnormal.

In some embodiments, a RBC membrane permeability parameter is scaled sphericity index (sSI). sSI is sphericity index (SI) multiplied by a scaling factor of 10. In some embodiments, a sSI of from about 14.2 to about 17.2, from about 14.7 to about 16.7, or from about 15.2 to about 16.2 is considered normal. In some embodiments, a sphericity index of about 14.7, about 15.2, about 15.7, about 16.2, or about 16.7 is considered normal. In some embodiments, a sphericity index of less than about 14.2, about 14.7, or about 15.2, or greater than about 16.2, about 16.7, or about 17.2 is considered abnormal. In some embodiments, a sphericity index of from about 10.0 to about 14.2, from about 10.0 to about 14.7, from about 10.0 to about 15.2, from about 16.2 to about 30.0, from about 16.7 to about 30.0, or from about 17.2 to about 30.0 is considered abnormal.

In some embodiments, a RBC membrane permeability parameter is slope between maximum and minimum points of the Fluid Flux Curve (slope_{FFC}). Slope_{FFC} is a measure of cell diversity and can be determined as described herein, e.g., from the Fluid Flux Curve of Example 1. In some embodiments, a slope_{FFC} of from about −1.7 (fL·10⁻¹)/(mOsm/kg)² to about 3.1 (fL·10⁻¹)/(mOsm/kg)², from about −0.9 (fL·10⁻¹)/(mOsm/kg)² to about 2.3 (fL·10⁻¹)/(mOsm/kg)², or from about −0.1 (fL·10⁻¹)/(mOsm/kg)² to about 1.5 (fL·10⁻¹)/(mOsm/kg)² is considered normal. In some embodiments, a slope_{FFC} of about −0.9 (fL·10⁻¹)/(mOsm/kg)², about −0.1 (fL·10⁻¹)/(mOsm/kg)², about 0.7 (fL·10⁻¹)/(mOsm/kg)², about 1.5 (fL·10⁻¹)/(mOsm/kg)², or about 2.3 (fL·10⁻¹)/(mOsm/kg)² is considered normal. In some embodiments, a slope_{FFC} of less than about −0.1 (fL·10⁻¹)/(mOsm/kg)², about −0.9 (fL·10⁻¹)/(mOsm/kg)², or about −1.7 (fL·10⁻¹)/(mOsm/kg)², or greater than about 1.5 (fL·10⁻¹)/(mOsm/kg)², about 2.3 (fL·10⁻¹)/(mOsm/kg)², or about 3.1 (fL·10⁻¹)/(mOsm/kg)² is considered abnormal. In some embodiments, a slope_{FFC} of from about −10 (fL·10⁻¹)/(mOsm/kg)² to about −1.7 (fL·10⁻¹)/(mOsm/kg)², from about −10 (fL·10⁻¹)/(mOsm/kg)² to about −0.9 (fL·10⁻¹)/(mOsm/kg)², from about −10 (fL·10⁻¹)/(mOsm/kg)² to about −0.1 (fL·10⁻¹)/(mOsm/kg)², from about 1.5 (fL·10⁻¹)/(mOsm/kg)² to about 10 (fL·10⁻¹)/(mOsm/kg)², from about 2.3 (fL·10⁻¹)/(mOsm/kg)² to about 10 (fL·10⁻¹)/(mOsm/kg)², or from about 3.1 (fL·10⁻¹)/(mOsm/kg)² to about 10 (fL·10⁻¹)/(mOsm/kg)² is considered abnormal.

In some embodiments, a RBC membrane permeability parameter is δ dynes. δ dynes is a measure of the force necessary to convert intact cells at their spherical volume to ghost cells at their spherical volume. In some embodiments, δ dynes is determined by measuring the difference between the most common cell size in the intact cell population at a particular osmolality and the most common cell size in the ghost cell population at a particular osmolality. In some embodiments, a δ dynes of from about 25 dynes to about 44 dynes, from about 28 dynes to about 41 dynes, or from about 31 dynes to about 38 dynes is considered normal. In some embodiments, a δ dynes of about 28 dynes, about 31 dynes, about 35 dynes, about 38 dynes, or about 41 dynes is considered normal. In some embodiments, a δ dynes of less than about 25 dynes, about 28 dynes, or about 31 dynes, or greater than about 38 dynes, about 41 dynes, or about 44 dynes is considered abnormal. In some embodiments, a δ dynes of from about 1 dynes to about 25 dynes, from about 1 dynes to about 28 dynes, from about 1 dynes to about 31 dynes, from about 38 dynes to about 100 dynes, from about 41 dynes to about 100 dynes, or from about 44 dynes to about 100 dynes is considered abnormal.

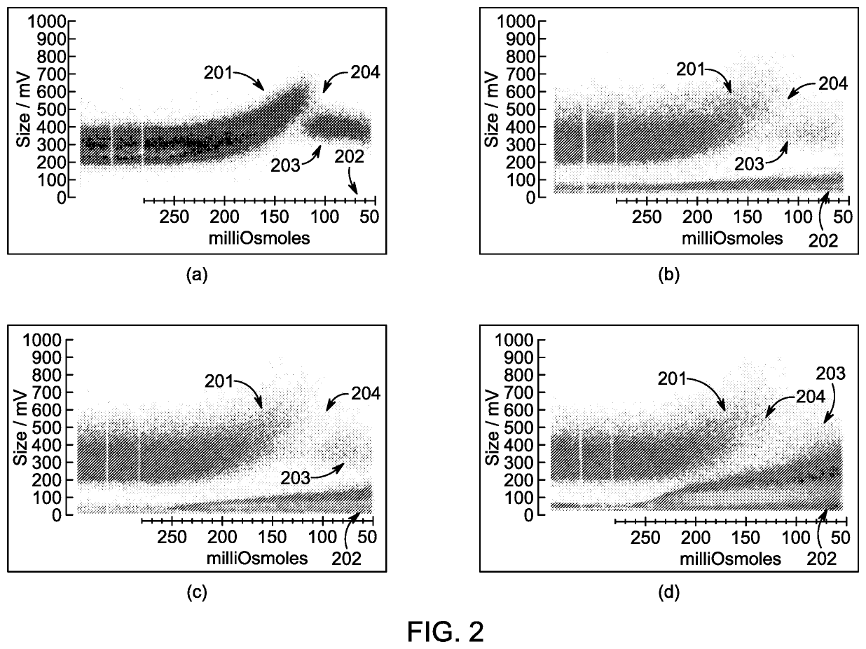

In some embodiments, a RBC membrane permeability parameter is fragmentation grade. Fragmentation grade is assigned on a scale of 0-3 as described in Example 1 and FIG. 2. In some embodiments, a fragmentation grade of from about 0 to about 1 or from about 0 to about 0.5 is considered normal. In some embodiments, a fragmentation grade of about 0, about 0.5, or about 1 is considered normal. In some embodiments, a fragmentation grade of greater than about 0.5, greater than about 1, or greater than about 1.5 is considered abnormal. In some embodiments, a fragmentation grade of from about to 0.5 to about 3, from about 1 to about 3, or from about 1.5 to about 3 is considered abnormal.

In some embodiments, a RBC membrane permeability parameter is fragmentation grade. Fragmentation grade is assigned on a scale of 0-6 as described in Example 10 and Table 9. In some embodiments, a fragmentation grade of from about 0 to about 2 or from about 0 to about 1 is considered normal. In some embodiments, a fragmentation grade of about 2, about 1, or about 0 is considered normal. In some embodiments, a fragmentation grade of greater than about 1, greater than about 2, or greater than about 3 is considered abnormal. In some embodiments, a fragmentation grade of from about 1 to about 6, from about to 2 to about 6, or from about 3 to about 6 is considered abnormal.

In some embodiments, a RBC membrane permeability parameter is Cell Scan shape. In some embodiments, Cell Scan shape is determined qualitatively. In some embodiments, Cell Scan shape is determined based on the number of features in common with a reference Cell Scan (e.g., a normal Cell Scan or an abnormal Cell Scan). In some embodiments, a qualitative determination of Cell Scan shape can comprise assigning a value from 1-20 based on the degree of variability from normal according to the scale described in Example 3. In some embodiments, a Cell Scan shape value of from about 1 to about 2 or from about 1 to about 1.5 is considered normal. In some embodiments, a Cell Scan shape value of about 1, about 1.5, or about 2 is considered normal. In some embodiments, a Cell Scan shape value of greater than about 1, about 2, about 3, about 4, or about 5, or more is considered abnormal. In some embodiments, a Cell Scan shape value of from about 1.5 to about 20, from about 2 to about 20, or from about 3 to about 20 is considered abnormal. In some embodiments, Cell Scan shape is determined quantitatively. For example, in some embodiments, the shape of the Cell Scan is fit using an appropriate function, such as a polynomial function, using e.g., a computer-implemented algorithm. In some such embodiments, the RBC membrane permeability parameter can be one or more coefficients of a polynomial function. Such coefficients can be compared to reference control parameters as described herein.

In some embodiments, Cell Scan shape provides additional information about a patient's health state and/or a patient's potential diagnosis. The present disclosure encompasses the recognition that one or more features of Cell Scan shape correspond with one or more particular diseases, disorders or conditions. It will be appreciated that Cell Scan shape is suggestive, though not necessarily definitive, of a particular health state. Nevertheless, this disclosure provides valuable insight related to Cell Scan shape. For example, while a normal curve shape is comparable to Cell Scan Shape N in FIG. 5, patients with a malignancy often exhibit some distortion and/or deviation from a normal Cell Scan shape. In some embodiments, a Cell Scan shape comparable to Cell Scan Shape L in FIG. 5 is suggestive of leukemia and/or lymphoma. In some embodiments, a Cell Scan shape comparable to Cell Scan Shape P in FIG. 5 is suggestive of pancreatic cancer and/or lung cancer. In some embodiments, a Cell Scan shape comparable to Cell Scan Shape G in FIG. 5 is suggestive of gastrointestinal tract malignancies, e.g., adenocarcinomas of the GI tract. In some embodiments, a Cell Scan shape comparable to Cell Scan Shape MF in FIG. 5 is suggestive of preleukemic stage myelodysplasia. In some embodiments, a Cell Scan shape comparable to Cell Scan Shape T in FIG. 5 is suggestive of beta thalassemia heterozygotes, hemoglobin S homozygotes, and/or hemoglobin C homozygotes. In some embodiments, a Cell Scan shape comparable to Cell Scan Shape HS in FIG. 5 is suggestive of hereditary spherocytosis and/or hemolytic anemias. In some embodiments, a Cell Scan shape comparable to Cell Scan Shape C in FIG. 5 is suggestive of liver disease and/or cirrhosis.

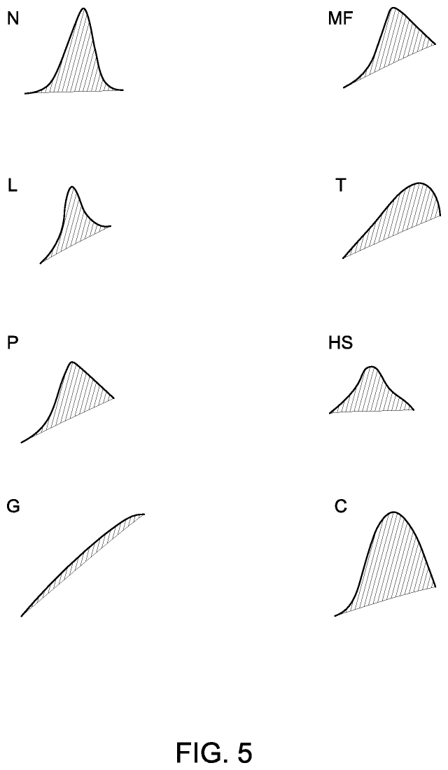

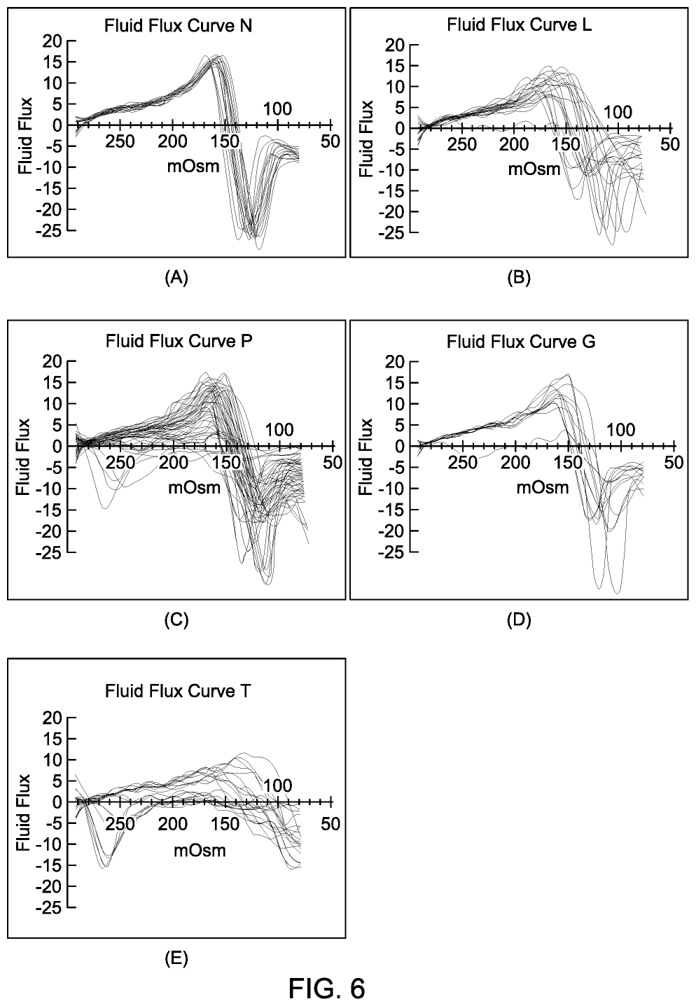

In some embodiments, Fluid Flux Curve (FFC) shape provides additional information about a patient's health state and/or a patient's potential diagnosis. The present disclosure encompasses the recognition that one or more features of FFC shape correspond with one or more particular diseases, disorders or conditions. It will be appreciated that FFC shape is suggestive, though not necessarily definitive, of a particular health state. Nevertheless, this disclosure provides valuable insight related to FFC shape. For example, while a normal curve shape is comparable to that of FIG. 6A, patients with a malignancy often exhibit some distortion and/or deviation from a normal FFC shape. In some embodiments, a Cell Scan shape comparable to that of FIG. 6B (i.e., FFC shape L) is suggestive of leukemia and/or lymphoma. In some embodiments, a FFC shape comparable to that of FIG. 6C (i.e., FFC shape P) is suggestive of pancreatic cancer and/or lung cancer. In some embodiments, a FFC shape comparable to that of FIG. 6D (i.e., FFC shape G) is suggestive of gastrointestinal tract malignancies, e.g., adenocarcinomas of the GI tract. In some embodiments, a FFC shape comparable to that of FIG. 6E (i.e., FFC shape T) is suggestive of beta thalassemia heterozygotes, hemoglobin S homozygotes, and/or hemoglobin C homozygotes.

In some embodiments, a RBC membrane permeability parameter is combined probability profile (CPP). CPP is an additive likelihood that a sample is normal or abnormal, calculated by adding together [(mean-value)/SD]² for each of the following parameters: Cp, Pk0, IsoV, SphV, Inc %, W10, Pxmin, Pxmax, Pymin, Pymax, Py ratio, sSI, slope_{FFC}, and ∂ dynes. In some embodiments, a CPP of from about 5.8 to about 15, from about 6.5 to about 12, or from about 7.0 to about 10 is considered normal. In some embodiments, a CPP of about 6.5, about 7.0, about 8.5, about 10, or about 12 is considered normal. In some embodiments, a CPP of less than about 7.0, about 6.5, or about 5.8, or greater than about 10, about 12, or about 15 is considered abnormal. In some embodiments, a CPP of from about 0 to about 5.8, from about to 0 to about 6.5, from about 0 to about 7.0, from about 10 to about 30, from about 12 to about 30, or from about 15 to about 30 is considered abnormal. In some embodiments, a CPP of from about 0.5 to about 8.5, from about 2.6 to about 5.4, or from about 2.5 to about 6.5 is considered normal. In some embodiments, a CPP of about 2.6, about 2.5, about 4.0, about 4.5, about 5.4, or about 6.5 is considered normal. In some embodiments, a CPP of less than about 2.6, about 2.5, or about 0.5, or greater than about 6.5, about 5.4, or about 8.4 is considered abnormal. In some embodiments, a CPP of from about 0 to about 0.5, from about to 0 to about 2.6, from about 0 to about 2.5, from about 8.5 to about 30, from about 5.4 to about 30, or from about 6.5 to about 30 is considered abnormal.

### Screening & Diagnosing Subject(s)

The present disclosure also provides methods of screening and/or diagnosing subjects using cell scanning technologies described herein.

In some embodiments, the present disclosure provides methods of identifying a subject in need of diagnostic assessment or therapeutic intervention. In some embodiments, a method of identifying a subject in need of diagnostic assessment or therapeutic intervention comprises steps of:
determining one or more RBC membrane permeability parameters from a sample of the subject's blood;
comparing the determined parameter to a reference control parameter selected from the group consisting of a positive reference control parameter, a negative reference control parameter, or both; and
identifying the subject as in need of when the determined parameter is not comparable to the negative reference control parameter and/or is comparable to the positive reference control parameter.

In some embodiments, the present disclosure provides methods of identifying a subject in no need of diagnostic assessment nor therapeutic intervention. In some embodiments, a method of identifying a subject in no need of diagnostic assessment nor therapeutic intervention comprises steps of:
determining one or more RBC membrane permeability parameters from a sample of the subject's blood;
comparing the determined parameter to a reference control parameter selected from the group consisting of a positive reference control parameter, a negative reference control parameter, or both; and
identifying the subject as not in need of when the determined parameter is not comparable to the negative reference control parameter and/or is comparable to the positive reference control parameter.

In some embodiments, a reference control parameter is a negative reference control parameter. For example, in some embodiments, a negative reference control parameter is obtained from a healthy individual or population of healthy individuals. In some embodiments, a negative reference control parameter is obtained from a population of healthy blood donors.

In some embodiments, a subject is identified as in need of diagnostic assessment or therapeutic intervention when the determined parameter is not comparable to the negative reference control parameter. In some embodiments, a determined parameter is not comparable to the negative reference control parameter when the determined parameter has a value that is at least 2%, 3%, 4%, 5%, 6%, 7%, 8%, 9%, 10%, 11%, 12%, 13%, 14%, 15%, 16%, 17%, 18%, 19%, or 20% different from the negative reference control parameter. In some embodiments, the determined parameter is not comparable to the negative reference control parameter when the determined parameter has a value that is 1, 2, 3, 4, 5, or more standard deviations away from the negative reference control parameter. In some embodiments, a determined parameter is not comparable to the negative reference control parameter when the determined parameter comprises one or more features that are not substantially similar to the negative reference control parameter.

In some embodiments, a reference control parameter is a positive reference control parameter. For example, a positive reference control parameter can be obtained from a subject or population of subjects suffering from a disease, disorder, or condition. In some embodiments, a positive reference control parameter is obtained from a subject or population of subjects suffering from a disease, disorder, or condition that is the same disease, disorder, or condition for which the subject is being screened.

In some embodiments, a subject is identified as in need of diagnostic assessment or therapeutic intervention when the determined parameter is comparable to the positive reference control parameter. In some embodiments, a determined parameter is comparable to the positive reference control parameter when the determined parameter has a value that is within 2%, 3%, 4%, 5%, 6%, 7%, 8%, 9%, 10%, 11%, 12%, 13%, 14%, 15%, 16%, 17%, 18%, 19%, or 20% of the positive reference control parameter. In some embodiments, the determined parameter is comparable to the positive reference control parameter when the determined parameter has a value that is within 1, 2, 3, 4, or 5 standard deviations of the positive reference control parameter. In some embodiments, a determined parameter is comparable to the positive reference control parameter when the determined parameter comprises one or more features that are substantially similar to the positive reference control parameter.

In some embodiments, identification of a subject as in need of diagnostic assessment or therapeutic invention can inform recommendations from medical professionals for further diagnostic assessment and/or therapeutic intervention. Accordingly, in some embodiments, provided methods further comprise performing diagnostic assessment and/or determining one or more clinical variables (e.g., when a subject is identified as in need of). In some embodiments, provided methods further comprise taking a medical history. In some embodiments, provided methods further comprise performing a physical examination. In some embodiments, provided methods further comprise performing one or more blood tests (e.g., CBC, blood protein testing such as haptoglobin levels, lactate dehydrogenase levels or hemoglobin electrophoresis, reticulocyte count, Coombs test, red cell survival test, liver function tests, circulating tumor cell tests, and tests for tumor markers such as prostate-specific antigen, cancer antigen 125, calcitonin, alpha-fetoprotein, and human chorionic gonadotropin). In some embodiments, provided methods further comprise performing one or more urine tests. In some embodiments, provided methods further comprise performing one or more genetic tests (e.g., to determine if a subject is likely to develop a particular inherited disease). In some embodiments, provided methods further comprise performing imaging (e.g., X-ray, CT scan, MRI, PET scan, etc.). In some embodiments, provided methods further comprise performing a biopsy (e.g., of bone, bone marrow, breast, esophagus, stomach, duodenum, rectum, colon, ileum, lung, liver, prostate, brain, nerve, meningeal, renal, endometrial, cervical, lymph node, muscle, or skin).

The present disclosure encompasses the recognition that one or more medications that the subject has taken or is taking may affect the output of the cell scanning technologies described herein. The effect of a particular medication on the output of the cell scanning technologies may vary by medication, e.g., vary in magnitude and/or in length of effect, and in some cases, a particular medication may have no effect at all. For example, phenobarbitone, penicillamine, phenytoin, and amphotericin B are medications that have an effect on the output of the cell scanning technologies described herein. Accordingly, in some embodiments, provided methods further comprise determining whether or not the subject has taken or is taking a particular medication. In some embodiments, if the subject has taken a medication known to have a particular effect on the output of the cell scanning technologies, then a determined RBC membrane permeability parameter should be compared to a suitable reference control parameter (e.g., a reference control parameter determined in the presence or absence of the particular medication). In some embodiments, if the subject has taken a medication known to have a particular effect on the output of the cell scanning technologies, then further diagnostic assessment is warranted.

The present disclosure also provides methods of diagnosing subjects (e.g., differentially diagnosing subjects) using RBC membrane permeability parameters described herein. In some embodiments, the present disclosure provides methods of diagnosing subjects who have been identified as in need of diagnostic assessment using a method described herein (e.g., a screening method described herein). In some embodiments, a method of diagnosing a subject with a disease, disorder, or condition comprises steps of:
determining one or more RBC membrane permeability parameters from a sample of the subject's blood;
comparing the determined parameters to a reference data set; and
calculating a probability that the subject has the disease, disorder, or condition.

In some embodiments, provided methods of diagnosing further comprise determining one or more clinical variables (e.g., age, gender, medical history, etc.) from the subject. In some embodiments, the determined clinical variables can be compared with a reference data set, separately or in combination with the determined RBC membrane permeability parameters. Suitable clinical variables will be known to those of skill in the art and may include those described herein.

In some embodiments, a reference data set comprises RBC membrane permeability parameters and/or clinical variables obtained from a plurality of subjects (e.g., healthy subjects and/or subjects for whom diagnosis of a particular disease, disorder, or condition has been confirmed). In some embodiments, a reference data set is organized by indication (e.g., organized so that the mean and/or median value for each parameter and/or variable is reported for each indication). In some embodiments, a reference data set is organized by indication and further by a range of values for a particular parameter and/or variable (e.g., organized so that the number of subjects with a value for the parameter and/or variable that falls within each range is reported). For example, suitable reference data sets are shown in Table 1-5 of Example 10.

In some embodiments, provided methods of diagnosing comprise calculating a probability (e.g., a quantitative probability) that a subject has a particular disease, disorder, or condition. Such a probability can be calculated by any suitable means apparent to those of skill in the art. In some embodiments, a probability is calculated using latent class analysis. Latent class analysis is described at http://www.john-uebersax.com/stat/. Tools for latent class analysis include Latent GOLD and CorExpress, are available from Statistical Innovations (https://www.statisticalinnovations.com).

In some embodiments, provided methods of diagnosing can be computer-implemented. Accordingly, in some embodiments, the present disclosure provides a computer system for implementing the methods provided herein. In some embodiments, the present disclosure provides a computer system for determining a probability (e.g., a quantitative probability) that a subject has a particular disease, disorder, or condition, the computer system (i) being adapted to receive input related to one or more RBC membrane permeability parameters determined from a sample of the subject's blood; (ii) optionally being further adapted to receive input relating to other clinical variables; (iii) comprising a processor for processing the received inputs by comparing them to a reference data set; and (iv) being adapted to display or transmit the probability.

In some embodiments, provided methods further comprise administering suitable therapy (e.g., when a subject is identified as in need of). Suitable therapy will depend on a subject's diagnosis and can be determined by a medical professional according to standard medical practices.

In some embodiments, provided methods are particularly suitable for identifying subjects who are suffering from cancer (e.g., bladder cancer, bone cancer, breast cancer, carcinoid cancer, common bile duct cancer, bronchial cancer, colon cancer, endometrial cancer, gall bladder cancer, ileum carcinoid carcinoma, leukemia, lung cancer, lymphoma such as Hodgkins and non-Hodgkins lymphoma, malignant melanoma, multiple myeloma, mycosis fungoides, ovarian cancer, pancreatic cancer, prostate cancer, rectal cancer, renal cancer, sarcoma, stomach cancer, testicular cancer, thyroid cancer, or uterine cancer). In some embodiments, provided methods are particularly suitable for identifying subjects who are suffering from pancreatic, lung, or brain cancer. In some embodiments, provided methods are particularly suitable for identifying subjects who are suffering from a hematological disease, disorder, or malignancy (e.g., anemias, hemoglobinopathies, sickle cell disease, or beta-thalassemia). In some embodiments, provided methods are particularly suitable for identifying subjects who are pregnant. In some embodiments, provided methods are particularly suitable for identifying subjects who are suffering from a disease, disorder, or condition selected from Table 7. In some embodiments, provided methods are particularly suitable for identifying subjects who are suffering from thalassemias, including subjects who are homozygotes or heterozygotes. In some embodiments, provided methods are particularly suitable for identifying subjects who are suffering from chronic renal failure (CRF), e.g., subjects who are suffering from CRF and are undergoing dialysis.

In some embodiments, provided methods are particularly suitable for subjects who are susceptible to a particular disease, disorder, or condition. In some embodiments, susceptibility to a particular disease, disorder, or condition is based on a variety of factors (e.g., risk factors, etc.) that would be apparent to a medical professional. In some embodiments, provided methods are particularly suitable for subjects who have a history of a particular disease, disorder, or condition (e.g., are in remission from one or more cancers). Alternatively or additionally, provided methods are particularly suitable for subjects who have a family history of a particular disease, disorder, or condition. Alternatively or additionally, provided methods are particularly suitable for subjects in which a genetic mutation and/or biomarker for a particular disease, disorder, or condition has been detected.

### Monitoring Subjects

The present disclosure also provides methods of monitoring subjects and/or samples (e.g., blood samples) using the cell scanning technologies described herein. Such methods may be useful for, e.g., monitoring health state over time and/or monitoring therapy and/or prophylaxis. The present disclosure also encompasses the recognition that provided methods may be particularly useful for monitoring a single subject over time. In such cases, increased accuracy and/or decreased variability is expected.

In some embodiments, a method comprises steps of:
determining one or more RBC membrane permeability parameters from each of a plurality of blood samples obtained at different time points from a single subject; and
comparing the determined one or more parameters from a first time point with that from at least one later time point;
wherein a significant change in the determined one or more parameters over time indicates a material change in the subject's health state.

In some embodiments, a method comprises steps of:
determining one or more RBC membrane permeability parameters from a blood sample obtained from a subject for whom the one or more RBC membrane permeability parameters has previously been obtained at least once; and
comparing the determined one or more parameters with the previously obtained one or more parameters,
wherein a significant change in the determined one or more parameters compared to the previously obtained one or more parameters indicates a material change in the subject's health state.

In some embodiments, a significant change in a determined RBC membrane permeability parameter is a change of 1%, 2%, 3%, 4%, 5%, 6%, 7%, 8%, 9%, 10%, 11%, 12%, 13%, 14%, 15%, 16%, 17%, 18%, 19%, or 20%, or greater. In some embodiments, a significant change in a determined RBC membrane permeability parameter is a change of 1, 2, 3, 4, or 5, or greater standard deviations. In some embodiments, a significant change in a determined RBC membrane permeability parameter is evident from a lack of substantial similarity in one or more features of the RBC membrane permeability parameter. It will be appreciated that if more than one RBC membrane permeability parameters are determined, a significant change in just one of those RBC membrane permeability parameters is sufficient to establish a significant change.

In some embodiments, a subject and/or sample is monitored at regular intervals, such as every day, every week, every month, every two months, every 6 months, every 12 months, etc. In some embodiments, the different time points are separated from one another by a reasonably consistent interval. In some embodiments, the different time points are separated from one another by a day, a week, a month, two months, six months, a year, or longer. In some embodiments, the previously obtained one or more RBC membrane permeability parameters were obtained, e.g., a day, a week, a month, two months, six months, a year, or longer before the determined one or more RBC membrane permeability parameters.

In some embodiments, a subject may be monitored before, during, and/or after a particular event (e.g., an event that increases or decreases the subject's susceptibility to a particular disease, disorder, or condition). For example, in some embodiments, a subject may be monitored before and after travel to a geographical area where there is an increased risk of contracting a particular disease, disorder or condition (e.g., travel to parts of Africa, Asia, Central America, South America, Haiti, Dominican Republic, and some Pacific islands increasing an individual's risk of contracting malaria, or travel to certain parts of the United States increasing an individual's risk of lead poisoning).

In some embodiments, methods provided herein may be useful for monitoring therapy and/or prophylaxis status and/or efficacy. In some embodiments, a subject may be monitored before and after initiation of therapy and/or prophylaxis. In some embodiments, therapy and/or prophylaxis is continued or discontinued based on the outcome of monitoring with provided methods. For example, in some embodiments, if a significant change is observed in one or more RBC membrane permeability parameters compared to a parameter obtained prior to initiation of therapy, then the therapy may be considered effective and continued or discontinued based on the recommendation of a medical professional. In some embodiments, if a significant change is not observed in one or more RBC membrane permeability parameters compared to a parameter obtained prior to initiation of therapy, then the therapy may be considered ineffective and continued or discontinued based on the recommendation of a medical professional. In some embodiments, if a significant change is observed in one or more RBC membrane permeability parameters compared to a parameter obtained prior to initiation of prophylaxis, then the prophylaxis may be considered not effective and continued or discontinued based on the recommendation of a medical professional. In some embodiments, if a significant change is not observed in one or more RBC membrane permeability parameters compared to a parameter obtained prior to initiation of prophylaxis, then the prophylaxis may be considered effective and continued or discontinued based on the recommendation of a medical professional.

In some embodiments, monitoring subjects and/or samples using the cell scanning technologies provided herein can inform recommendations from medical professionals for further diagnostic assessment and/or therapeutic intervention. Accordingly, in some embodiments, provided methods further comprise performing diagnostic assessment and/or determining one or more clinical variables (e.g., when a significant change is or is not observed). Suitable diagnostic assessments are described above.

In some embodiments, provided methods further comprise administering suitable therapy (e.g., when a significant change is or is not observed). Suitable therapy will depend on a subject's diagnosis and can be determined by a medical professional according to standard medical practices. Suitable therapy is described above.

### Monitoring Blood Samples

In some embodiments, provided methods are particularly useful for evaluating viability of RBCs (e.g., stored blood samples). Blood that has been donated is typically stored for a defined period of time (e.g., 6 weeks) before being considered unfit for use. The present disclosure encompasses the recognition that the methods provided herein may be used to identify stored blood samples that are viable (e.g., viable beyond the standard expiration date), thereby extending how long a particular blood sample may be used and avoiding unnecessary waste of blood samples (e.g., donated blood samples). In some embodiments, provided methods may also be used to identify samples with reduced viability before the standard expiration date, thereby preventing administration of blood with reduced viability. In some embodiments, a method comprises steps of:
determining one or more RBC membrane permeability parameters from a sample of RBCs;
comparing the determined parameter to a reference control parameter selected from the group consisting of a positive reference control parameter, a negative reference control parameter, or both; and
identifying the sample of RBCs as not viable when the determined parameter is not comparable to the negative reference control parameter and/or is comparable to the positive reference control parameter.

In some embodiments, a reference control parameter is a negative reference control parameter. For example, in some embodiments, a negative reference control parameter is obtained from a viable sample or a plurality of viable samples of RBCs.

In some embodiments, a sample of RBCs is identified as not viable when the determined parameter is not comparable to the negative reference control parameter. In some embodiments, a determined parameter is not comparable to the negative reference control parameter when the determined parameter has a value that is at least 2%, 3%, 4%, 5%, 6%, 7%, 8%, 9%, 10%, 11%, 12%, 13%, 14%, 15%, 16%, 17%, 18%, 19%, or 20% different from the negative reference control parameter. In some embodiments, the determined parameter is not comparable to the negative reference control parameter when the determined parameter has a value that is 1, 2, 3, 4, 5, or more standard deviations away from the negative reference control parameter. In some embodiments, a determined parameter is not comparable to the negative reference control parameter when the determined parameter comprises one or more features that are not substantially similar to the negative reference control parameter.

In some embodiments, a reference control parameter is a positive reference control parameter. For example, a positive reference control parameter can be obtained from a sample or plurality of samples of RBCs that are not viable.

In some embodiments, a sample of RBCs is identified as not viable when the determined parameter is comparable to the positive reference control parameter. In some embodiments, a determined parameter is comparable to the positive reference control parameter when the determined parameter has a value that is within 2%, 3%, 4%, 5%, 6%, 7%, 8%, 9%, 10%, 11%, 12%, 13%, 14%, 15%, 16%, 17%, 18%, 19%, or 20% of the positive reference control parameter. In some embodiments, the determined parameter is comparable to the positive reference control parameter when the determined parameter has a value that is within 1, 2, 3, 4, or 5 standard deviations of the positive reference control parameter. In some embodiments, a determined parameter is comparable to the positive reference control parameter when the determined parameter comprises one or more features that are substantially similar to the positive reference control parameter.

In some embodiments, a sample of RBCs has been stored for a period of time (e.g., about 1 day, about 1 week, about 2 weeks, about 3 weeks, about 4 weeks, about 5 weeks, about 6 weeks, about 10 weeks, about 14 weeks, about 6 months, etc.). In some embodiments, a sample of RBCs was obtained from a blood donor (e.g., a healthy blood donor).

In some embodiments, provided methods further comprise repeated evaluation of a sample of RBCs over time (e.g., in order to monitor when a sample of blood expires, i.e., is no longer viable). In some embodiments, a sample of RBCs is evaluated every day, every week, every 2 weeks, every 3 weeks, or every month.

In some embodiments, provided methods further comprise administering a sample of RBCs that has been identified as viable to a subject in need thereof. In some embodiments, provided methods further comprising not administering a sample of RBCs that has been identified as not viable or as having reduced viability to a subject in need thereof. In some embodiments, provided methods further comprise disposing of a sample of RBCs that has been identified as not viable.

### Predicting Life Expectancy

The present disclosure also provides methods of predicting life expectancy (e.g., likelihood that a subject will die within a particular time period) using the cell scanning technologies described herein. In some embodiments, a method comprises steps of:
determining one or more RBC membrane permeability parameters from a sample of a subject's blood;
comparing the determined parameter to a reference control parameter selected from the group consisting of a positive reference control parameter, a negative reference control parameter, or both; and
identifying a subject as likely to die within a time period when the determined parameter is not comparable to the negative reference control parameter and/or is comparable to the positive reference control parameter.

In some embodiments, a reference control parameter is a negative reference control parameter. For example, in some embodiments, a negative reference control parameter is obtained from a healthy individual or population of healthy individuals. In some embodiments, a negative reference control parameter is obtained from a population of healthy blood donors.

In some embodiments, a subject is identified as likely to die within a time period when the determined parameter is not comparable to the negative reference control parameter. In some embodiments, a determined parameter is not comparable to the negative reference control parameter when the determined parameter has a value that is at least 2%, 3%, 4%, 5%, 6%, 7%, 8%, 9%, 10%, 11%, 12%, 13%, 14%, 15%, 16%, 17%, 18%, 19%, or 20% different from the negative reference control parameter. In some embodiments, the determined parameter is not comparable to the negative reference control parameter when the determined parameter has a value that is 1, 2, 3, 4, 5, or more standard deviations away from the negative reference control parameter. In some embodiments, a determined parameter is not comparable to the negative reference control parameter when the determined parameter comprises one or more features that are not substantially similar to the negative reference control parameter.

In some embodiments, a reference control parameter is a positive reference control parameter. For example, a positive reference control parameter can be obtained from a subject or population of subjects who have died within a particular time period.

In some embodiments, a subject is identified as not likely to die within a time period when the determined parameter is comparable to the positive reference control parameter. In some embodiments, a determined parameter is comparable to the positive reference control parameter when the determined parameter has a value that is within 2%, 3%, 4%, 5%, 6%, 7%, 8%, 9%, 10%, 11%, 12%, 13%, 14%, 15%, 16%, 17%, 18%, 19%, or 20% of the positive reference control parameter. In some embodiments, the determined parameter is comparable to the positive reference control parameter when the determined parameter has a value that is within 1, 2, 3, 4, or 5 standard deviations of the positive reference control parameter. In some embodiments, a determined parameter is comparable to the positive reference control parameter when the determined parameter comprises one or more features that are substantially similar to the positive reference control parameter.

In some embodiments, a subject is identified as likely to die within a time period of about 36 months, about 48 months, about 60 months, about 72 months, about 84 months, about 96 months, about 108 months, about 120 months, or longer.

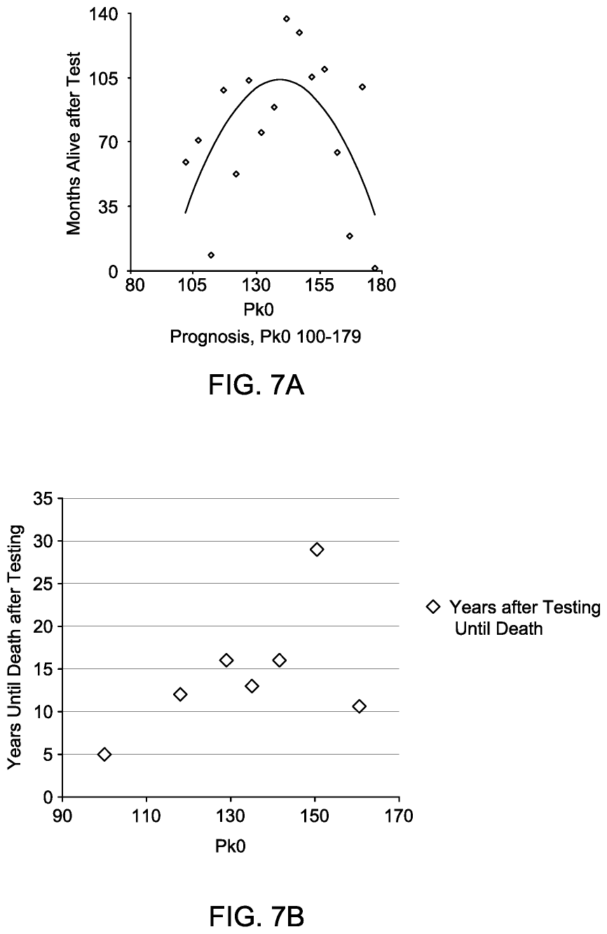

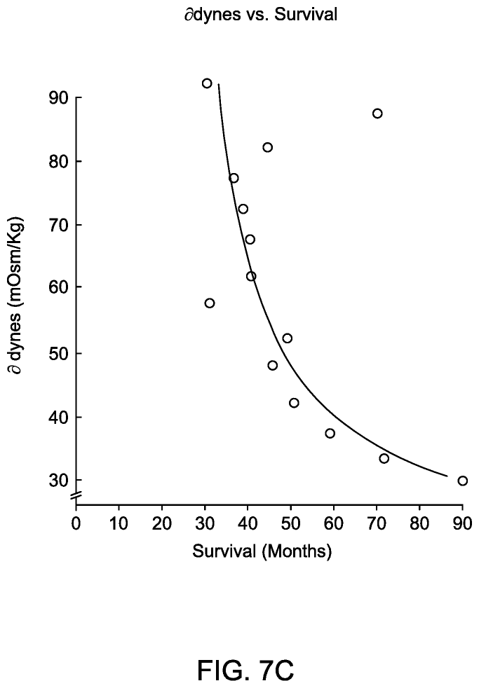

The present disclosure also encompasses the recognition that a RBC membrane permeability parameter (e.g., Pk0, ∂ dynes, or CPP) may, in some embodiments, be particularly useful for predicting life expectancy. In some embodiments, a plot of a RBC membrane permeability parameter vs. months alive after test is useful as a standard curve for predicting life expectancy. In some embodiments, provided methods comprise comparing a determined Pk0 value to a standard curve (e.g., FIG. 7A or FIG. 7B) and identifying a subject as likely to die within a particular time period. In some embodiments, provided methods comprise comparing a determined CPP value to a standard curve and identifying a subject as likely to die within a particular time period. In some embodiments, provided methods comprise comparing a determined ∂ dynes value to a standard curve (e.g., FIG. 7C) and identifying a subject as likely to die within a particular time period.

In some embodiments, provided methods useful for predicting life expectancy may be computer-implemented. Accordingly, in some embodiments, the present disclosure provides a computer system for implementing the methods provided herein. In some embodiments, the present disclosure provides a computer system for determining a probability (e.g., a quantitative probability) that a subject is likely to die within a time period, the computer system (i) being adapted to receive input related to one or more RBC membrane permeability parameters determined from a sample of the subject's blood; (ii) optionally being further adapted to receive input relating to other clinical variables; (iii) comprising a processor for processing the received inputs by comparing them to a reference data set; and (iv) being adapted to display or transmit the probability.

Identification and/or Characterization of RBC Permeability Modulating Agents and/or Compositions

The present disclosure also provides technologies for assessing (e.g., identifying and/or characterizing) agents and/or other compositions that modulate RBC membrane permeability (collectively, “RBC Permeability Modulating Agents”). Provided technologies may be useful for identifying RBC Permeability Modulating Agents. In some instances, a RBC Permeability Modulating Agent is expected to effect the health state of a subject exposed it (e.g., a beneficial or adverse effect). For example, provided methods allow for the evaluation of agents and/or compositions intended for use in humans (e.g., a drug candidate or prosthetic material). In some embodiments, a method comprises:
contacting a sample of blood from a healthy subject with an agent or composition;
determining one or more RBC membrane permeability parameters from the sample of blood;
comparing the determined parameter to a reference control parameter selected from the group consisting of a positive reference control parameter, a negative reference control parameter, or both; and
identifying the agent or composition as a RBC Permeability Modulating Agent when the determined parameter is not comparable to the negative reference control parameter and/or is comparable to the positive reference control parameter.

In some embodiments, a reference control parameter is a negative reference control parameter. For example, in some embodiments, a negative reference control parameter is obtained from a healthy individual or population of healthy individuals. In some embodiments, a negative reference control parameter is obtained from a population of healthy blood donors.

In some embodiments, a sample of RBCs is identified as a RBC Permeability Modulating Agent when the determined parameter is not comparable to the negative reference control parameter. In some embodiments, a determined parameter is not comparable to the negative reference control parameter when the determined parameter has a value that is at least 2%, 3%, 4%, 5%, 6%, 7%, 8%, 9%, 10%, 11%, 12%, 13%, 14%, 15%, 16%, 17%, 18%, 19%, or 20% different from the negative reference control parameter. In some embodiments, the determined parameter is not comparable to the negative reference control parameter when the determined parameter has a value that is 1, 2, 3, 4, 5, or more standard deviations away from the negative reference control parameter. In some embodiments, a determined parameter is not comparable to the negative reference control parameter when the determined parameter comprises one or more features that are not substantially similar to the negative reference control parameter.

In some embodiments, a reference control parameter is a positive reference control parameter. For example, a positive reference control parameter can be obtained from a sample or plurality of samples of RBCs with modulated RBC membrane permeability.

In some embodiments, a sample of RBCs is identified as RBC Permeability Modulating Agent when the determined parameter is comparable to the positive reference control parameter. In some embodiments, a determined parameter is comparable to the positive reference control parameter when the determined parameter has a value that is within 2%, 3%, 4%, 5%, 6%, 7%, 8%, 9%, 10%, 11%, 12%, 13%, 14%, 15%, 16%, 17%, 18%, 19%, or 20% of the positive reference control parameter. In some embodiments, the determined parameter is comparable to the positive reference control parameter when the determined parameter has a value that is within 1, 2, 3, 4, or 5 standard deviations of the positive reference control parameter. In some embodiments, a determined parameter is comparable to the positive reference control parameter when the determined parameter comprises one or more features that are substantially similar to the positive reference control parameter.

In some embodiments, a sample is analyzed within a particular time period after being subjected to an agent or composition (e.g., within about 5 minutes, about 10 minutes, about 30 minutes, about 1 hour, about 2 hours, or about 5 hours). In some embodiments, a method further comprises evaluating dose response of an agent or composition (e.g., by subjecting each of a plurality of samples to varying concentrations of agent or composition).

In some embodiments, a method provided herein further comprises utilizing (e.g., administering or contacting) an agent and/or composition that has been identified as not a RBC Permeability Modulating Agent. In some embodiments, a method provided herein further comprises not utilizing (e.g., not administering or not contacting) an agent and/or composition that has been identified as a RBC Permeability Modulating Agent. In some embodiments, a method provided herein further comprises additional assessment of an agent and/or composition identified as a RBC Permeability Modulating Agent (e.g., further safety assessment). It will be appreciated that an appropriate risk-benefit analysis will be warranted when an agent and/or composition is identified as a RBC Permeability Modulating Agent.

### EXAMPLES

### Example 1. Cell Scan for Cell Membrane Permeability

A sample of whole blood from a healthy volunteer was drawn into ACD anticoagulant. The unwashed sample was divided into aliquots and was analyzed using the Prior Shine Technology and/or the Provided Cell Scanning Technologies. The following outputs were obtained from the sample:

### Cell-by-Cell Color Map

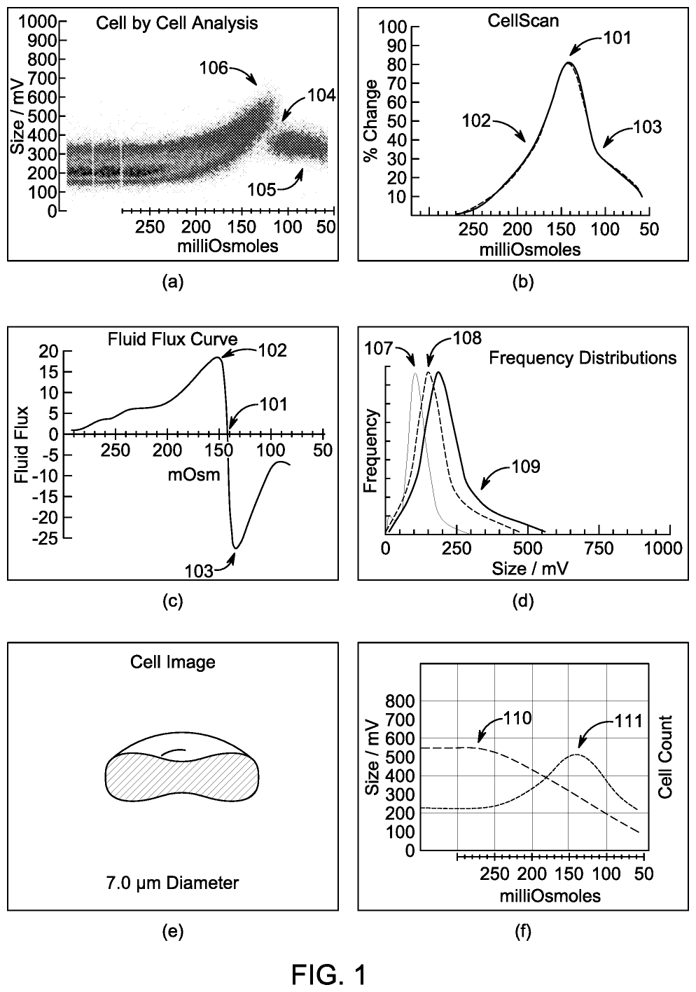

Cell membrane permeability recorded on a cell-by-cell basis is shown in FIG. 1a. The number of blood cells within each aliquot were counted (typically, e.g., at least 1000), and the cell-by-cell data was then used to produce an exact frequency distribution of cell permeability. Frequency distributions of each sample are conveniently displayed using different colors (e.g., a color map), as shown in FIG. 1a. In a cell-by-cell graph, population density is represented by color, with zero density corresponding to white, the lowest nonzero density corresponding to the darkest points (e.g., blue), and, as density progressively increases, color of the points lightens (e.g., from green to yellow to orange to red to black to aqua).

One feature of the cell-by-cell graph is the portion of the graph associated with intact cells (e.g., from about 300 mOsm/kg to about 70 mOsm/kg); during this period, the size of the cell population does not change, and thereafter, the cell population increases in volume, and then falls. The static initial period is the result of cell's exposure to isotonic fluid, and the remainder is the result of exposure to progressive increase in osmotic stress.

“Pk0” coincided with the minimum absolute osmotic pressure (e.g., most hypotonic pressure) to which a cell can be subjected without loss of integrity. Pk0 can be identified by determining the right-most extent of the intact cell population in the cell-by-cell graph, i.e., the point of osmolality immediately preceding the point at which the cells ruptured. In FIG. 1a, this minimum pressure is the “peak” 106. As the osmolality of the surrounding solution was reduced, the red blood cell ruptures and forms a ghost cell, which releases its contents into the surrounding medium.

In the cell-by-cell graph, there typically appears to the right of the expanding intact cell (EIC) population, a second and smaller cluster. This smaller cluster comprises “ghost cells,” which are cells that have ruptured and thereafter resealed themselves (labeled 105 in FIG. 1a). Between the EIC population and the ghost cell cluster appears a relatively colorless or cell free area, termed the “ghost gap” (labeled 104 in FIG. 1a). The presence of a ghost gap is normal for cells of healthy individuals and is diminished or absent for individuals with certain types of conditions.

Another feature in the cell-by-cell graph is a region associated with the presence of cell fragments, which have a smaller volume (e.g., an average volume of about 20 fL) and therefore appear at the bottom of the graph, above the baseline (202 in FIG. 2) and toward the right. Cell fragments (i.e., schistocytes) are differentiated by their relatively small size and response to osmotic stress (e.g., increase in size and/or number under osmotic stress). As the osmolality of the surrounding solution was reduced, fragments appeared to increase in size by about 70% and increased in number by about 200%. For a healthy individual, the cell-by-cell graph showed few, if any, cell fragments. For unhealthy individuals, the cell-by-cell graph displayed a larger population of cell fragments, which increased in size with the increase in osmotic stress. In some embodiments, severity of cell fragmentation can be ranked on a scale of zero (no fragments) through 3 (most severe), or from low to moderate to severe as shown in FIG. 2. In some embodiments, an actual count of cell fragments is provided.

A third feature of the cell-by-cell graph is a region associated with the presence of platelets, located below the standard curve and immediately above the baseline. Platelets are characterized by their smaller size (e.g., a mean volume of about 10 fL). In some embodiments, platelets do not increase significantly when subjected to decreasing osmolality, and the population size of platelets does not increase with osmotic stress. For a healthy individual, the cell-by-cell graph showed a normal platelet population just above the baseline. A larger population of platelets was observed, though, in individuals with, for example, certain infections, hemoglobinopathies, tuberculosis, rheumatoid arthritis, and cancers.

Percent Cell Volume Change Vs. Osmolality (“Cell Scan Plot”)

Using the technologies described herein, a cell-by-cell analysis was converted into a plot of percent change of cell volume vs. osmolality (“Cell Scan Plot”) by converting the individual peak voltage into a cell volume, then calculating a mean volume for each 100 cells, and plotting the means to generate the Cell Scan Plot. The percentage change of cell volume at each osmolality is calculated and compared to the mean cell volume of an isotonic cell (e.g., FIG. 1b). On such a plot, Pk0 (see 101) is the osmotic pressure at which the net water flow is zero (i.e., when a cell achieved its maximum volume, i.e., when it is a perfect sphere). As described herein, in some embodiments, Pk0 can be used as an indicator of an individual's health status.

### Fluid Flux Curve (FFC)

The Fluid Flux Curve (FFC) was determined by taking the first order derivative (with respect to osmolality) of Cell Scan Plot (FIG. 1c). In an FFC, Pk0 occurred at the zero crossing (101), which was where the slope of the Cell Scan Plot changes from positive to negative. A positive value on the FFC represented a net flow of fluid into the cell, while negative rates represented a net flow of fluid out of the cell. In the FFC, the positive peak 102 and negative peak 103 corresponded to the maximum and minimum, respectively, on the FFC. As used herein, “Pymax” is the magnitude of fluid flux at the maximum, and “Pymin” is the magnitude of fluid flux at the minimum.

From the values of Pk0, Pymax, and Pymin, a cell size and shape were estimated, as shown in FIG. 1e. In FIG. 1e, the depiction of a red blood cell at the isotonic osmolality is scaled to size.

### Frequency Distribution of Cell-by-Cell Analysis

The frequency distribution of the cell-by-cell analysis, as shown in FIG. 1d, was determined from the cell-by-cell plot of FIG. 1a. The frequency distribution is a classical density distribution of red blood cell population and was examined at different osmolalities to calculate statistical parameters including the mean, the standard deviation, coefficient of variation, normality, skewness, kurtosis, and the number of inflection points. As shown in FIG. 1d, three distributions are depicted, which correspond to the three “cuts” on the cell-by-cell curve (FIG. 1a). These “cuts” correspond to the distribution at three osmolality values: the solid thin line 107 being isotonic (resting) cells (i.e., 280 mOsm/kg), bold line 109 being spherical cells (i.e., 142 mOsm/kg), and dotted line 108 being ghost cells (i.e., 110 mOsm/kg). It will be appreciated that the “cuts” can be made at any point along the cell-by-cell plot, and a frequency distribution plotted for each of them.

### Raw Data Curve

An exemplary “Raw Data Curve” is shown in FIG. 1f, which shows superimposed graphs of mean voltage 111 and cell count 110 for a scan against osmolality. As shown, the cell count, which was initially relatively high at the beginning of the scan, reduced throughout the test due to the dilution of the sample using the cell scanning technologies described herein. The mean voltage rose to a maximum at a critical osmolality, where the red blood cells achieved a spherical shape, and then reduced. In some embodiments, a Raw Data Curve, such as the one in FIG. 1f, can be used to confirm that a suitable osmolality gradient was achieved during the course of the RBC permeability measurement. In some embodiments, a suitable osmolality gradient is substantially linear.

### Scattering

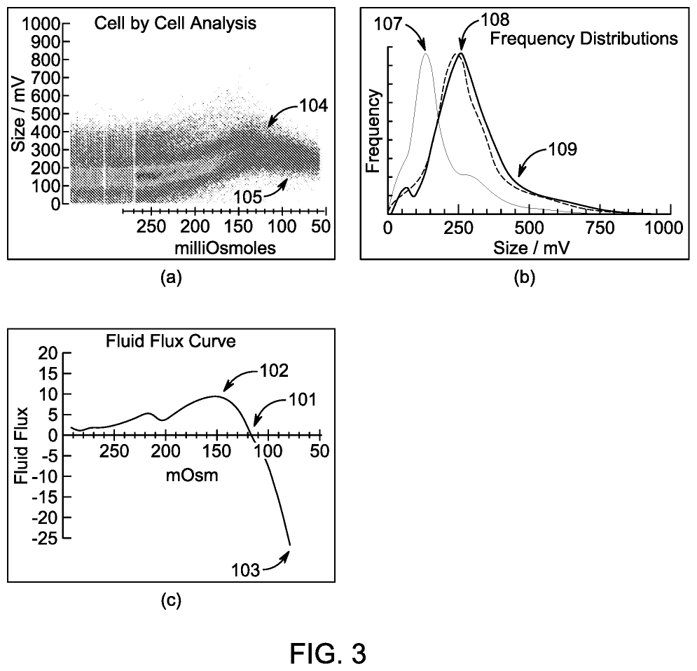

Scattering (i.e., cell heterogeneity or cell diversity) was measured in at least six ways, including intensity of color on the cell-by-cell graph (FIG. 3a), size of the ghost gap (FIG. 3a), standard deviation on the Frequency Distribution Curve (FIG. 3b), number of inflection points (jaggedness) on any of the Frequency Distribution Curves (FIG. 3b), the wobbliness of the FFC (FIG. 3c), and peak width at 10% below maximum peak height (W10) of the Cell Scan Plot.

### Sphericity Index

Sphericity index is measured as described in WO 97/24601. In some embodiments, sphericity index is multiplied by a scaling factor (e.g., a scaling factor of 10). A sphericity index multiplied by a scaling factor of 10 is referred to herein as a scaled sphericity index (sSI).

### Example 2. Exemplary Cell Scans of a Patient in an Unhealthy State

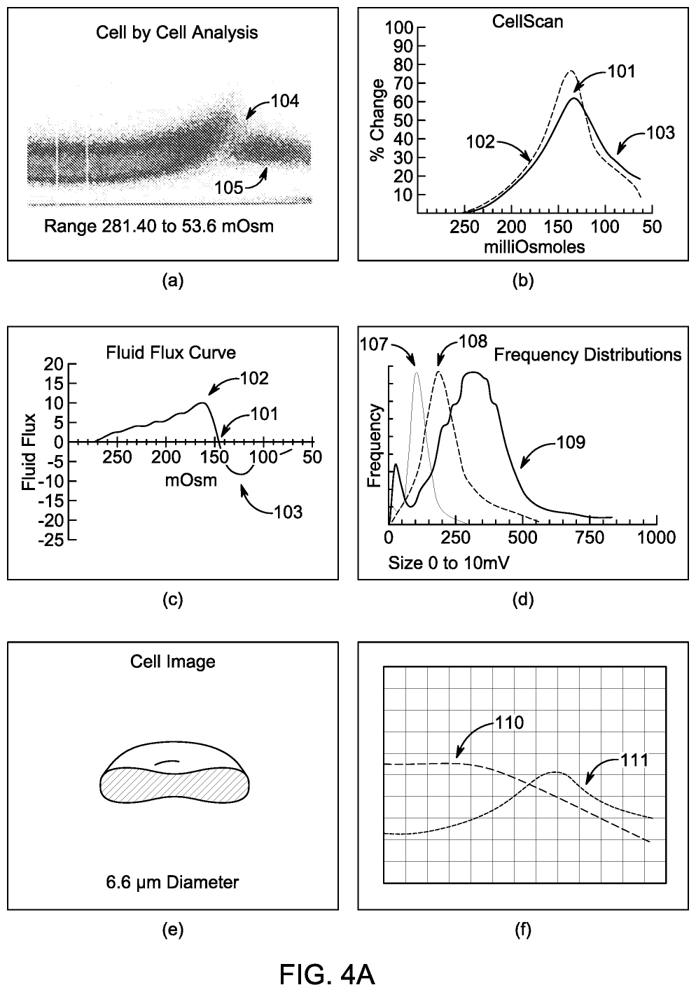

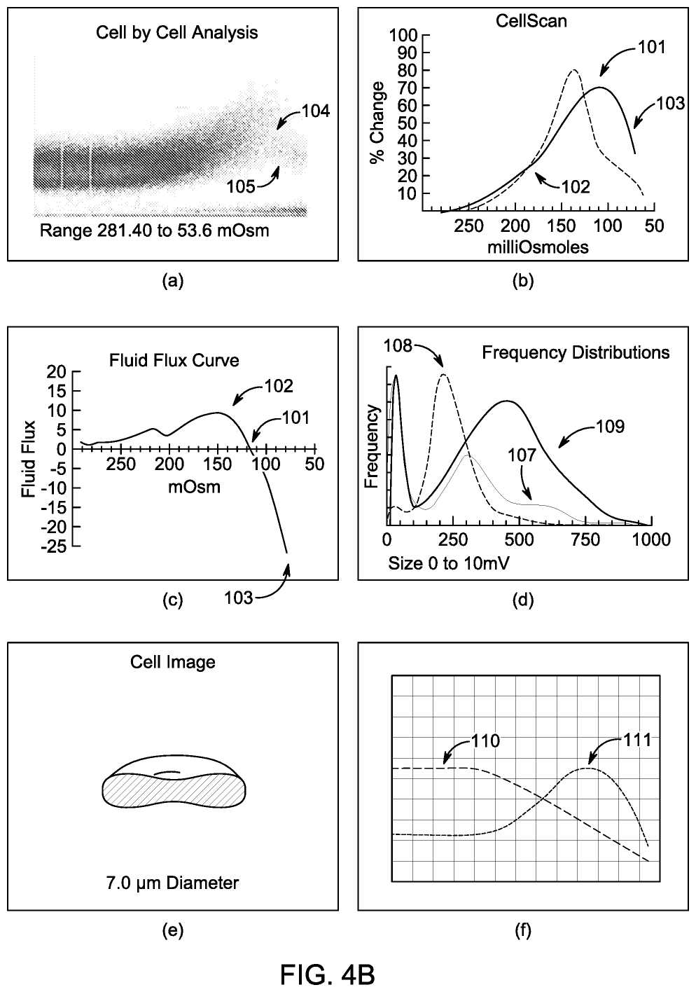

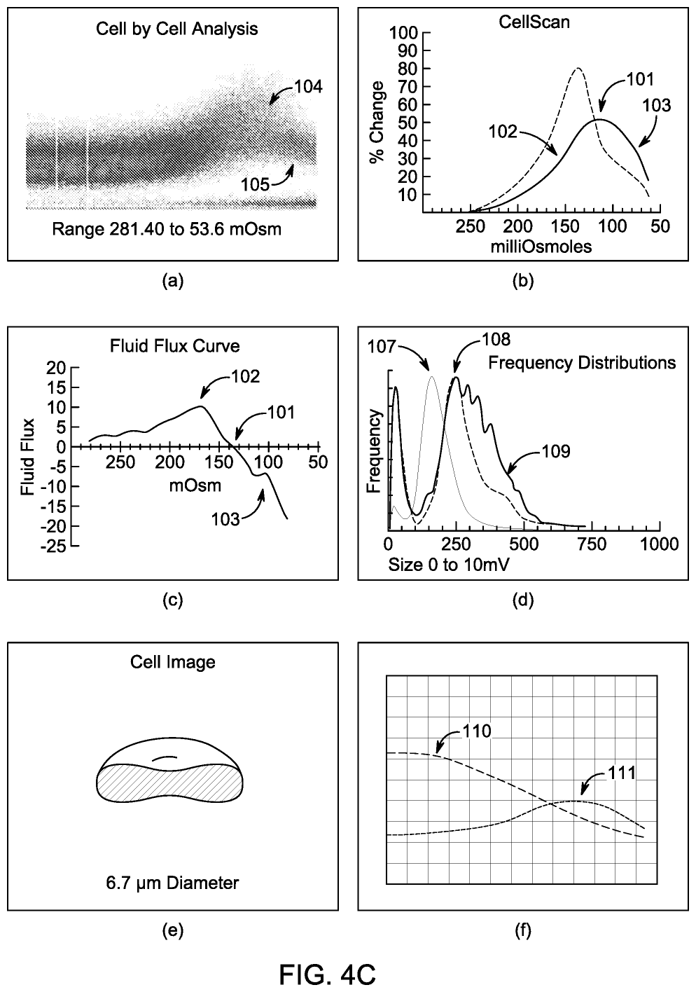

Any or all of the parameters described in Example 1 can be used to evaluate the health status of a patient. In some embodiments, a shift in one or more of the parameters described in Example 1 is indicative of an unhealthy state in said patient. FIGS. 4A-4D are exemplary cell scanner outputs from patients in an unhealthy state. When compared to FIG. 1, which depicts cell scanner outputs from a healthy individual, several differences were observed in FIGS. 4A-4D. It will be appreciated that FIGS. 4A-4D are merely representative of cell scanner outputs from patients in an unhealthy state and are not intended to be limiting in any way. In fact, the present disclosure encompasses the recognition that a shift in any one of the parameters described herein (e.g., Pk0, Pymin, Pymax, scattering, sphericity index, shape of Cell Scan curve, platelet count, fragment count, percentage size increase, slope of fluid flux curve, etc.) may be indicative of an unhealthy state of the patient. In some embodiments, certain parameters may be particularly indicative of an unhealthy state of a patient in the early stages of disease, such as Pymin, Pymax, percentage size increase, slope of fluid flux curve, etc.).

FIG. 4A depicts a cell scanner output from a patient diagnosed with cancer of unknown primary origin. As can be seen in FIG. 4A, in comparison to the sample from a healthy patient shown in FIG. 1, the FFC was compressed (i.e., the magnitude of Pymin and Pymax is reduced), some scattering was observed in the cell-by-cell plot, and the frequency distribution was jagged (e.g., 109).

FIG. 4B depicts a cell scanner output from a patient diagnosed with cirrhosis. As can be seen in FIG. 4B, in comparison to the sample from a healthy patient shown in FIG. 1, the cell-by-cell graph displays very few ghost cells (105), Pk0 (101) is shifted to approx. 118 mOsm/kg, and the curve shapes of the Cell Scan Plot, the FFC, and the frequency distribution are all abnormal.

FIG. 4C depicts a cell scanner output from a patient diagnosed with malignancy of unknown origin. As can be seen in FIG. 4C, in comparison to the sample from a healthy patient shown in FIG. 1, the cell-by-cell graph does not display a ghost gap (104), Pk0 (101) is shifted to approx. 135 mOsm/kg, and the curve shapes of the Cell Scan Plot, the FFC, and the frequency distribution are all abnormal.

FIGS. 4A-4C clearly demonstrate that even small deviations in any one of the cell permeability parameters described herein are considered significant to an evaluation of a patient's health status. Deviations, particularly between samples from the same patient, e.g., over the course of time, are almost always indicative of development of an unhealthy state for the patient.

### Example 3. Diagnostic Screening Technology Based on Cell Membrane Permeability

Based on the results of, e.g., Example 2, a statistical analysis was performed on a larger data set to validate the diagnostic value of the insights provided herein. First, a control set was used to establish normal ranges for four parameters using blood from healthy volunteers. Then, the normal ranges were verified using a test set, comprising samples of blood from patients with a prior diagnosis of disease. The results from the test set were positive and confirmed that at least the four parameters evaluated were suitable for use in a diagnostic screening system, as provided herein.

### Control Set—Healthy Volunteers

A group of 275 consecutive blood donors was used as a control set for the purpose of evaluating the provided diagnostic screening technologies. Blood donors are generally considered representative of a healthy population. For each sample in the control set, four parameters were compared: Pk0, SphV, IsoV, and CS Shape. It was noted that inclusion of two additional parameters (presence of fragments and presence of platelets) did not change the outcome of the analysis.

Pk0 was determined as described in Example 1.

The spherical volume (SphV) was derived from the voltage measured using provided cell scanning technologies at Pk0.

The isotonic volume (IsoV) was calculated as derived from the voltage measured using provided cell scanning technologies at the initial osmolality.

The shape of the Cell Scan curve (CS shape) was assigned a number from 1-20 based on the degree of variability from normal according to the following scale:

| | |
|---|---|
| 1 | Normal, based on compilation of data from healthy blood donors |
| 2-5 | Pk0 within normal range, CS shape slightly wider and/or shorter |
| | than normal (e.g., FIG. 4A) |
| 6-10 | Pk0 shifted, CS shape moderately abnormal (e.g., FIG. 4C) |
| 10-20 | Pk0 greatly shifted, CS shape grossly abnormal (e.g., FIG. 4B) |

The following results were obtained from the control set of samples which were drawn into ACD, and are considered normal values for the purposes of this Example:
Pk0: mean=146.33 mOsm/kg, SD=5.6
SphV: mean=170.06 femtoliters, SD=11.776
IsoV: mean=91.13 femtoliters, SD=5.149
CS Shape: 1

The following results were obtained from the control set of samples which were drawn into EDTA:
Pk0: mean=144.1 mOsm/kg, SD=5.9
SphV: mean=163.8 femtoliters, SD=12.6
IsoV: mean=89.8 femtoliters, SD=6.1
CS Shape: 1

Among other things, the present disclosure establishes control reference values for relevant parameter(s) (e.g., for one or more RBC membrane permeability parameters).

Test Set—Patients with Prior Diagnosis

A test set of 793 patients diagnosed with a malignancy via other methods was then compiled for comparison with the control set. This set of 793 samples was tested blindly using provided cell scanning technologies and compared to the control set of samples from normal, healthy volunteers. A binary classification was used to mark samples from the test set as “normal” or “abnormal”. If any one of the four parameters (i.e., Pk0, SphV, IsoV, or CS Shape) fell outside of the normal range, the sample was considered “abnormal”. A sample was considered “abnormal” if it met any one of the following:
Pk0<mean−q*SD
SphV<mean−q*SD
IsoV>mean+q*SD
CS shape>1

Using the data from the control and test sets, the sensitivity and specificity were calculated to evaluate the provided technologies as a screening tool. For this analysis, a normal population of 275 subjects and a test population of 793 subjects with a malignancy were used. The results are shown below in Table 1 and demonstrate that the provided technologies successfully differentiate samples from healthy individuals and those with a malignancy:

TABLE 1

| q | Sensitivity | Specificity |
|---|---|---|
| 0.84 | 87.8% | 57.8% |
| 1.28 | 81.8% | 78.2% |
| 1.64 | 74.5% | 87.3% |
| 2 | 71.5% | 94.5% |

Various subsets of the test set were also evaluated, compared to the control set. In particular, three subsets of patients were analyzed using the statistical analysis described above: those with pancreatic malignancy, lung malignancy, and brain malignancy. Notably, reliable and convenient screening tests do not currently exist for any of these types of malignancy. Provided cell scanning technologies were shown to successfully detect each type of malignancy compared to the control set. Results are summarized in Table 2 below:

TABLE 2

| Malignancy | N | q | Sensitivity | Specificity |
|---|---|---|---|---|
| Pancreas | 19 | 2 | 84.2% | 94.5% |
| Lung | 110 | 2 | 61.8% | 94.5% |
| Brain | 19 | 2 | 64.3% | 94.5% |

The results described herein, e.g., in Example 3, indicate that the provided cell scanning technologies are relevant for use a diagnostic screening tool. The provided diagnostic screening technologies are as good, if not better, than other routine screening technologies. For example, Table 3 summarizes the sensitivity and specificity of representative routine screening technologies:

TABLE 3

| Routine Screen | Sensitivity | Specificity |
|---|---|---|
| Provided Technology¹ | ~61-84% | 94.5% |
| Mammogram² | 79% | 95% |
| Fecal Occult³ | 92% | 87% |
| Pap Smear⁴ | 68% | 78% |

¹Calculated using data from three subsets of patients, as described in Table 2.
²https://www.cancer.gov/types/breast/hp/breast-screening-pdq, accessed on 2019 Oct. 28.
³https://www.cologuardtest.com/hcp, accessed on 2019 Oct. 28 https://www.cancer.gov/types/cervical/hp/cervical-screening-pdq, accessed on 2019 Dec. 1.

### Diagnosis of Patients of Unknown Status

Based on the success of the analysis of the control and test sets described above, blood donors of unknown status were screened. In one experiment, 1500 volunteer blood donors were screened, all of whom reported no symptoms and were presumed healthy. Of the 1500 patients, 99.5% returned normal cell scanner outputs. The remaining patients, however, upon further investigation by clinicians, were determined to have a malignancy or other serious pathology. Thus, the provided diagnostic screening technologies allowed for the early diagnosis of a disease state, which may have otherwise gone unnoticed.

In another experiment, individuals who had been given a relatively benign diagnosis from a physician were evaluated using the provided diagnostic screening technologies. In several cases, the provided technologies indicated that a sample was “abnormal”. Upon further testing of patients with an “abnormal” sample, such patients were found to indeed have a more serious disease/pathology, which would have gone undetected for a longer period of time in the absence of the provided cell scanning technologies. Table 4 provides representative examples of early detection using the provided technologies but is not intended to be limiting in any way:

TABLE 4

| | Eventual Dx after having been |
|---|---|
| Original Dx by other clinicians | flagged by the scanner |
| perforation of gut | malignancy of pancreas |
| abdo mass | malignancy of endometrium |
| hematuria and duodenal ulcer | lymphoma |
| Blood clotting problem | malignancy of colon |
| obstructive jaundice | malignancy of gall bladder |
| pelvic abscess perhaps* | malignancy of colon |
| no dx | malignancy of colon |
| probable lymphoma | lymphoma |
| obstructive jaundice | malignancy of gall bladder |
| R flank pain & fever | malignancy of bladder |
| jaundice secondary to gallstones | cancer of UKP |
| no dx | cancer of UKP |
| PUO (fever of unknown origin) | malignancy of prostate |
| for arteriogram | |
| rectovescicle fistula | malignancy of bladder |
| bleeding per rectum, no known | malignancy of colon |
| cause | |
| intestinal obstruction | malignancy of colon sigmoid |
| intestinal obstruction | malignancy of rectum |
| recurrent anemia hiatus hernia | malignancy of stomach |
| no dx | malignancy of ileum carcinoid |
| | carcinoma |
| intestinal obstruction | malignancy of stomach |
| anemia | acute myeloleukemia |
| intestinal obstruction acute | malignancy of stomach |
| emoyemia post cholecystectomy | malignancy of bronchus |

*UKP = unknown primary origin

### Example 4. Evaluating Agents and/or Other Compositions Prior to In Vivo Use

Using the technologies provided herein, agents were tested for potential adverse in vivo effects prior to use in humans and/or animals. For example, a blood sample from a healthy volunteer was contacted with amphotericin B (0.5 μg/mL) and was analyzed using the provided cell scanning technologies. A Pk0 of 85 mOsm/kg was observed, indicating a shift from normal Pk0 (i.e., approx. 142 mOsm/kg). Similarly, all new drugs could be tested using our technology prior to patient exposure to predict and/or avoid potentially adverse reactions. Alternatively or additionally, provided technologies can be used to evaluate materials used in a clinical setting (e.g., polymers used for medical implants, or e.g., prosthetic heart valve components).

### Example 5. Monitoring Treatment Status of Patients

The treatment status of patients who have undergone therapy (e.g., chemotherapy or cancer removal surgery) can be evaluated and monitored using the technologies provided herein. Results from testing a blood sample obtained from a patient prior to therapy and a blood sample obtained from the same patient after therapy (and optionally at regular intervals thereafter) are compared. Prior to therapy, patients are expected to exhibit “abnormal” results as described herein. If the therapy successfully treats the patient's condition, results are expected to be “normal” as described herein. If the therapy is not successful (in whole or in part), results are expected to be “abnormal” as described herein.

### Example 6. Assessing Life Expectancy

A study was conducted examining subjects for whom a date of death was confirmed (N=1586) to determine if cell permeability was indicative of life expectancy. FIG. 7A shows a graph plotting months alive after Cell Scan vs. Pk0. Each data point in FIG. 7A represents mean duration of life for patients with that Pk0 value. As shown in FIG. 7A, cell membrane permeability is related to life expectancy, and in particular, the greater the deviation from a normal Pk0 (i.e., approx. 142 mOsm/kg in this Example), the shorter the life expectancy of the patient. A similar graph is shown in FIG. 7C for ∂ dynes. Accordingly, the present disclosure encompasses the recognition that cell membrane permeability is a reliable measure of the remaining duration of a patient's life.

Furthermore, on a subset of the population of subjects with a confirmed date of death, e.g., those who were pregnant at the time of the Cell Scan, a similar relationship was observed (FIG. 7B), which suggests the predictive value of cell membrane permeability parameters extends to a variety of patient populations.

### Example 7. Monitoring Blood Banks

Provided technologies were utilized to monitor the viability of donor blood over time. Donated blood is typically stored for a defined period of time (i.e., 6 weeks) before being considered unfit for use. Yet, using the technologies described herein, donated blood was monitored over time and was shown to be viable, in most cases, for longer than 6 weeks.

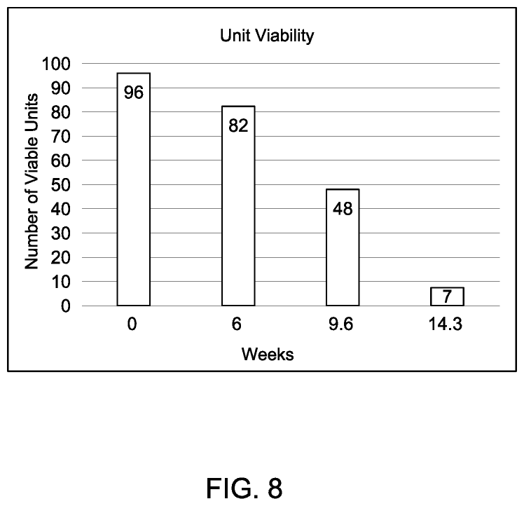

Over the course of 100 days, 96 units of donated blood were tested using the technologies described herein. Approximately every 7-10 days, a sample of blood from each unit was taken, held at room temperature for an hour, diluted with phosphate buffered saline to a concentration of 0.5 million RBCs/mL and analyzed using the provided cell scanning technologies. Samples were considered “not viable” if Pk0, SphV, IsoV, or Cell Scan shape fell outside of “normal” range, as described in Example 3. As shown in FIG. 8, after 6 weeks, almost all blood samples (˜85%) still exhibited normal measurements for, e.g., Pk0. Further, a small percentage of blood samples (˜7%) maintained viability throughout the entire 100 day period. This experiment demonstrates that the provided technologies can be used to evaluate if donated blood is still usable, thereby minimizing waste and potentially providing viable blood to patients in need.

### Example 8. Identifying RBC Fragments

Provided technologies also enable the detection and counting of RBC fragments in a blood sample, which has traditionally been difficult and taken several days to test, due to the variety of shapes and sizes observed. Using the technologies provided herein, however, RBC fragment counts can be obtained readily.

For example, as shown in Table 5, we have detected fragments in patients with hemolytic-uremic syndrome (HUS); glomerulonephritis; renal graft rejection; vasculitis; malignant hypertension; metastatic carcinoma; heart valve hemolysis from pathological or prosthetic valves; severe burns; and HELLP syndrome, and ranked their fragmentation on a severity scale from 0 (least severe) to 3 (most severe).

TABLE 5

| Disease or condition | Fragment score |
|---|---|
| HUS | 0.6 |
| glomerulonephritis | 0.2 |
| Renal graft rejection | 0.9 |
| Vasculitis | 0.5-1 |
| Malignant hypertension | 0.2 |
| Metastatic carcinoma | 0.5-1.8 |
| Heart valve hemolysis | 1.4 |
| Severe burns | 0.5 |
| HELLP | 2 |

Fragments have been observed in other indications as well, such as thrombotic microangiopathy (TMA), which includes disseminated intravascular coagulation (DIC) and thrombotic thrombocytopenic purpura (TTP); cardiac anomalies; and march hemoglobinuria.

### Example 9. Evaluating Pregnancy

Over the course of 771 pregnancies, 1128 blood samples were analyzed for RBC fragment count, among other parameters, using the provided cell scanning technologies. In pregnant women, RBC fragment count increases with the term of the pregnancy, as described in Table 6. Shifts in other RBC membrane permeability parameters are also observed.

| | Coefficient of | | Sphericity | Fragment |
|---|---|---|---|---|
| Week | Permeability (Cp) | Pk0 | Index (SI) | Count |
| <32 | 2.3 ± 0.35 | 142.9 ± 7.5 | 1.72 | 10,000 |
| 32-<38 | 2.29 | 145 | 1.72 | 20,000 |
| 38-<40 | 2.07 | 150 | 1.68 | 40,000 |
| In labor | 2.07 | 150 | 1.68 | 60,000 |
| Post-natal | 2.3 | 145 | 1.74 | 20,000 |

In 14% of the pregnant women tested, a doubling of the fragment count was observed, which was suspected to be related to venous thromboembolism. It was also observed that in patients with ectopic pregnancies and/or high blood pressure in pregnancy, deviation from normal was detected for one or more cell membrane permeability parameters. Additionally, one patient out of 771 patients tested was identified as having HELLP syndrome using the technologies provided herein, based on her increased RBC fragment count and observed hemolysis.

### Example 10. Differential Diagnostic System Based on Cell Membrane Permeability

### Library of Diagnostic Data

Using the methods described herein, a library of diagnostic data has been constructed, some of which is summarized in Table 7 in Appendix C. Table 7 is organized by indication and reports the mean values for various parameters tested.

Across the approx. 280 diseases listed in Table 7, a few general trends are worth noting: (1) Mean Pk0 was typically lower in the subset of patients who had died from any particular disease, relative to those that had not died. (2) In indications with a hemolytic etiology, Pk0 was generally higher than in indications without a hemolytic etiology. (3) Pk0 gives an indication of basic pathology in diseases where RBC membrane permeability may not otherwise be indicated. For example, in patients with gall stones, provided technologies can provide an indication of associated hemolysis. (4) Across mechanistically related indications, such as hemoglobinopathies, Pk0 decreases with increasing clinical severity.

The present disclosure encompasses the recognition that a library of data obtained using technologies provided herein, such as the one described by Table 1 or Table 12, may be used as the foundation of a differential diagnostic system in addition to the screening application described above. By comparing parameters, such as those in Table 1, a probability value can be identified for each indication and can be used as a tool for differential diagnosis, either alone or in combination with other screening technologies.

### Latent Class Modeling

A differential diagnostic system is created, in which an individual's Cell Scan output is compared with data from a library (such as that described above) comprising parameters derived from provided cell scanning technologies including, but not limited to, e.g., Pk0, sphericity volume, isotonic volume, Cell Scan shape, scattering, fragment count, platelet count, Pymin, and/or Pymax, etc., and, optionally, additional parameters such as age, gender, and/or medical history. Based on the comparison, a probability that the individual has one or more indications is calculated, and a diagnosis is thus provided. The library may be stored within a computer and/or instrument, thus allowing software, or other suitable means, to generate a probability that the individual has a certain disease state for a given value of each parameter.

For example, library data may be organized as shown in Table 1, or as shown in Tables 8-11. Tables 8-11 summarize by indication mean values for Pk0, fragmentation count (on a scale of 0-6), scattering, and sphericity, respectively. Probabilities may be calculated using latent class analysis (or latent class modeling), given that the system would have one variable that is categorical (i.e., indication), as opposed to continuous. Latent class analysis is described at http://www.john-uebersax.com/stat/. Tools for latent class analysis include Latent GOLD and CorExpress, are available from Statistical Innovations (https://www.statisticalinnovations.com).

TABLE 8
Normal mean Pk0 value 142.3 SD +/− 5.7

| Pk0 | <90 | 90-99 | 100-109 | 110-119 | 120-124 | 125-154 | 155-159 | 160-169 | 170+ |
|---|---|---|---|---|---|---|---|---|---|
| N | 18 | 41 | 107 | 212 | 186 | | 510 | 320 | 70 |
| Disease Group | | | | | | | | | |
| Malignancy | 1 | 5 | 9 | 17 | 29 | | 80 | 59 | 17 |
| HB/Thal | 10 | 24 | 59 | 124 | 41 | | 6 | 2 | 1 |
| GI disease | 0 | 0 | 5 | 9 | 8 | | 25 | 22 | 5 |
| Pregnancy | 1 | 0 | 3 | 4 | 7 | | 93 | 103 | 24 |
| Blood disease | 1 | 4 | 7 | 14 | 15 | | 38 | 31 | 4 |
| (non-anemia) | | | | | | | | | |
| Blood disease | | | | | | | | | |
| (anemia only) | | | | | | | | | |
| Cardiovascular | 0 | 0 | 1 | 5 | 5 | | 46 | 28 | 4 |
| disease | | | | | | | | | |
| Central nervous | 0 | 0 | 1 | 1 | 0 | | 12 | 4 | 0 |
| system disease | | | | | | | | | |
| Collagen | 0 | 0 | 0 | 0 | 0 | | 1 | 0 | 0 |
| diseases | | | | | | | | | |
| Endocrine | 0 | 0 | 0 | 0 | 0 | | 0 | 0 | 0 |
| diseases | | | | | | | | | |
| Geriatric | 0 | 0 | 0 | 0 | 0 | | 0 | 0 | 0 |
| Infections | 0 | 0 | 0 | 3 | 2 | | 10 | 9 | 3 |
| Injuries | 0 | 0 | 0 | 1 | 1 | | 6 | 8 | 0 |
| Liver and biliary | 1 | 0 | 2 | 6 | 5 | | 2 | 2 | 2 |
| disease | | | | | | | | | |
| Metabolic | 0 | 0 | 1 | 0 | 0 | | 0 | 2 | 0 |
| disease | | | | | | | | | |
| Musculoskeletal | 1 | 1 | 1 | 2 | 3 | | 30 | 7 | 0 |
| disease | | | | | | | | | |
| Neonat | 3 | 4 | 9 | 4 | 24 | | 10 | 7 | 1 |
| Nutrition | 0 | 0 | 0 | 0 | 0 | | 0 | 0 | 0 |
| OB/Gyn | 0 | 0 | 0 | 1 | 2 | | 8 | 3 | 0 |
| Psychiatric | 0 | 0 | 1 | 0 | 0 | | 0 | 0 | 0 |
| Disease | | | | | | | | | |
| Pulmonary | 0 | 0 | 1 | 4 | 18 | | 22 | 13 | 2 |
| disease | | | | | | | | | |
| Renal disease | 0 | 1 | 4 | 16 | 13 | | 33 | 8 | 1 |
| Sarcoid | 0 | 0 | 1 | 1 | 0 | | 0 | 0 | 0 |
| Skin disease | 0 | 0 | 0 | 0 | 0 | | 0 | 0 | 0 |
| Surgery | 0 | 0 | 0 | 0 | 0 | | 0 | 1 | 0 |
| UG disease | 0 | 0 | 0 | 0 | 1 | | 7 | 0 | 2 |
| No diagnosis | 0 | 1 | 2 | 0 | 8 | | 9 | 5 | 1 |
| listed | | | | | | | | | |
| Blood donor | 0 | 1 | 0 | 0 | 2 | | 66 | 0 | 1 |
| Control | 0 | 0 | 0 | 0 | 2 | | 6 | 6 | 2 |

TABLE 9
Normal scans have fragmentation below grade 2

| | | | | | |
|---|---|---|---|---|---|
| Fragmentation | <2 | 2 | 3 | 4 | 5, 6 |

| | | | |
|---|---|---|---|
| N | 118 | 28 | 10 |
| Disease Group | | | |
| Malignancy | 21 | 6 | 2 |
| HB/Thal | 24 | 0 | 1 |
| GI disease | 2 | 0 | 0 |
| Pregnancy | 12 | 1 | 1 |
| Blood disease (non anemia) | 2 | 0 | 0 |
| Blood disease (anemia only) | 1 | 1 | 1 |
| Cardiovascular disease | 21 | 3 | 1 |
| Central nervous system | 1 | 1 | 0 |
| disease | | | |
| Collagen diseases | 0 | 0 | 0 |
| Endocrine diseases | 0 | 0 | 1 |
| Geriatric | 0 | 0 | 0 |
| Infections | 0 | 0 | 0 |
| Injuries | 0 | 0 | 0 |
| Liver and biliary disease | 1 | 0 | 0 |
| Metabolic disease | 0 | 0 | 0 |
| Musculoskeletal disease | 6 | 1 | 0 |
| Neonat | 1 | 0 | 0 |
| Nutrition | 0 | 0 | 0 |
| OB/Gyn | 0 | 1 | 0 |
| Psychiatric Disease | 0 | 0 | 0 |
| Pulmonary disease | 16 | 13 | 2 |
| Renal disease | 4 | 0 | 1 |
| Sarcoid | 1 | 0 | 0 |
| Skin disease | 0 | 0 | 0 |
| Surgery | 0 | 0 | 0 |
| UG disease | 0 | 0 | 0 |
| No diagnosis listed | 3 | 0 | 0 |
| Blood donor | 2 | 1 | 0 |
| Control | 0 | 0 | 0 |

TABLE 10
Normal controls have a mean value of 25 ± 4 SD

| Scattering | | | | | | | |
|---|---|---|---|---|---|---|---|
| (calculated as W10) | <15 | <20 | <25 | <30 | <35 | <40 | 40+ |
| N | 14 | 315 | 340 | 116 | 89 | 62 | 34 |
| Disease Group | | | | | | | |
| Malignancy | 0 | 79 | 76 | 25 | 27 | 21 | 6 |
| HB/Thal | 1 | 2 | 5 | 4 | 2 | 5 | 5 |
| GI disease | 2 | 40 | 26 | 7 | 5 | 2 | 2 |
| Pregnancy | 5 | 23 | 9 | 2 | 0 | 1 | 2 |
| Blood disease | 3 | 23 | 23 | 9 | 1 | 3 | 3 |
| (non-anemia) | | | | | | | |
| Blood disease | 0 | 43 | 49 | 20 | 19 | 11 | 8 |
| (anemia) | | | | | | | |
| Cardiovascular | 0 | 12 | 28 | 11 | 3 | 5 | 1 |
| disease | | | | | | | |
| Central nervous | 0 | 0 | 7 | 2 | 3 | 1 | 0 |
| system disease | | | | | | | |
| Collagen disease | 0 | 4 | 2 | 0 | 0 | 0 | 0 |
| Endocrine disease | 0 | 3 | 0 | 0 | 1 | 0 | 0 |
| Geriatric | 0 | 0 | 0 | 0 | 0 | 0 | 0 |
| Infection | 0 | 8 | 7 | 1 | 1 | 0 | 0 |
| Injury | 0 | 5 | 3 | 1 | 2 | 1 | 0 |
| Liver and biliary | 1 | 4 | 13 | 4 | 1 | 0 | 2 |
| disease | | | | | | | |
| Metabolic disease | 0 | 0 | 8 | 1 | 0 | 0 | 0 |
| Musculoskeletal | 0 | 14 | 18 | 2 | 2 | 3 | 1 |
| disease | | | | | | | |
| Neonat | 0 | 6 | 3 | 3 | 3 | 3 | 2 |
| Nutrition | 0 | 0 | 1 | 0 | 0 | 0 | 0 |
| OB/Gyn | 1 | 4 | 11 | 4 | 3 | 1 | 0 |
| Psychiatric disease | 0 | 0 | 1 | 0 | 1 | 0 | 0 |
| Pulmonary disease | 0 | 2 | 5 | 6 | 4 | 1 | 0 |
| Renal disease | 0 | 10 | 8 | 1 | 6 | 2 | 1 |
| Sarcoid | 0 | 0 | 0 | 0 | 0 | 0 | 0 |
| Skin disease | 0 | 0 | 0 | 0 | 0 | 0 | 0 |
| Surgery | 0 | 7 | 3 | 4 | 1 | 1 | 1 |
| UG disease | 0 | 4 | 4 | 1 | 2 | 0 | 0 |
| No diagnosis listed | 0 | 0 | 0 | 0 | 0 | 0 | 0 |
| Blood donor | 0 | 0 | 3 | 2 | 2 | 1 | 0 |
| Control | 1 | 22 | 27 | 6 | 0 | 0 | 0 |

TABLE 11
Normal mean value 1.77 ± 0.06 SD

| | | | | | | | More disc |
|---|---|---|---|---|---|---|---|
| | More spherical cells | | | | NORMAL RANGE | | shaped cells |
| SI | 1.3-1.39 | 1.4-1.49 | 1.5-1.59 | 1.6-1.69 | 1.7-1.79 | 1.8-1.89 | 1.9-2.1 |
| N | 10 | 13 | 58 | 310 | 442 | 227 | 54 |
| Disease Group | | | | | | | |
| Malignancy | 1 | 0 | 3 | 17 | 11 | 3 | 1 |
| HB/Thal | 1 | 4 | 20 | 49 | 52 | 23 | 15 |
| GI disease | 1 | 0 | 0 | 0 | 49 (45 | 54 (all 54 | 24 (all 24 |
| | | | | | of these | celiac) | |
| | | | | | are celiac) | | |
| Pregnancy | 1 | 1 | 14 | 150 | 153 | 31 | 1 |
| Blood disease | 0 | 0 | 6 | 8 | 8 | 1 | 1 |
| (non-anemia) | | | | | | | |
| Blood disease | 3 | 3 | 4 | 33 | 25 | 17 | 4 |
| (anemia only) | | | | | | | |
| Cardiovascular | 0 | 1 | 0 | 14 | 18 | 1 | 0 |
| disease | | | | | | | |
| Central nervous | 0 | 0 | 0 | 2 | 0 | 0 | 0 |
| system disease | | | | | | | |
| Collagen | 0 | 0 | 0 | 0 | 0 | 0 | 0 |
| disease | | | | | | | |
| Endocrine | 0 | 0 | 0 | 0 | 1 | 0 | 0 |
| disease | | | | | | | |
| Geriatric | 0 | 0 | 0 | 0 | 0 | 0 | 0 |
| Infection | 0 | 0 | 0 | 2 | 6 | 0 | 1 |
| Injury | 0 | 0 | 0 | 0 | 0 | 0 | 0 |
| Liver and | 0 | 0 | 1 | 4 | 0 | 1 | 0 |
| biliary disease | | | | | | | |
| Metabolic | 0 | 0 | 0 | 0 | 0 | 0 | 0 |
| disease | | | | | | | |
| Musculoskeletal | 0 | 0 | 1 | 0 | 10 | 6 | 1 |
| disease | | | | | | | |
| Neonat | 0 | 0 | 3 | 2 | 4 | 0 | 0 |
| Nutrition | 0 | 0 | 0 | 0 | 0 | 0 | 0 |
| OB/Gyn | 1 | 0 | 0 | 0 | 5 | 9 | 0 |
| Psychiatric | 0 | 0 | 2 | 0 | 0 | 0 | 0 |
| disease | | | | | | | |
| Pulmonary | 0 | 0 | 1 | 15 | 53 | 30 | 2 |
| disease | | | | | | | |
| Renal disease | 0 | 1 | 0 | 6 | 6 | 1 | 0 |
| Sarcoid | 0 | 0 | 0 | 0 | 0 | 0 | 0 |
| Skin disease | 0 | 0 | 0 | 0 | 0 | 0 | 0 |
| Surgery | 0 | 0 | 0 | 0 | 0 | 0 | 0 |
| UG disease | 0 | 0 | 0 | 0 | 0 | 1 | 0 |
| No diagnosis | 0 | 1 | 0 | 1 | 5 | 6 | 0 |
| listed | | | | | | | |
| Blood donor | 0 | 2 | 3 | 3 | 0 | 0 | 0 |
| Control | 2 | 0 | 0 | 4 | 36 | 43 | 4 |

### Example 11. Case Studies

### Patient Z

A 45 year old black man was admitted to the hospital for cholecystectomy. Prior to surgery, a sickledex test was performed and indicated that he was positive for the sickle cell trait, but given his age and mild anemia, the surgeon assumed that he was only a carrier for sickle cell anemia. Post operatively he developed severe pain. A sample of his blood was tested using cell scanning technologies described herein and revealed abnormal results such as: Cp=5.9 mL/m²; Pk0=107 mOsm/kg; IsoV=94 fL; SphV=177 fL; Inc %=88%; W10=30 mOsm/kg; Pxmin=73 mOsm/kg; Py ratio=0.8; sSI=15.8; slope_{FFC}=0.5 (fL·10⁻¹)/(mOsm/kg)²; ∂ dynes=65 dynes, Pxmax 137 mOsm/kg. Based on these results and his African heritage, sickle cell anemia was suspected. The patient was thus diagnosed with sickle cell anemia, not just a carrier of sickle cell trait.

### Patient Y

A 58 year old man with a persistent non-productive cough was admitted to a hospital where a blood sample was taken and tested using cell scanning technologies described herein. The results revealed abnormal results such as a Cp=3.7 mL/m²; Pk0=137 mOsm/kg; IsoV=88 fL; SphV=147 fL; Inc %=68%; W10=22 mOsm/kg; Pxmin=193 mOsm/kg; Pymax=9.6 (fL·10⁻¹)/mOsm/kg; Pymin=−12.6 (fL·10⁻¹)/mOsm/kg; Py ratio=0.8; sSI=16.8; slope_{FFC}=−0.7 (fL·10⁻¹)/(mOsm/kg)²; ∂ dynes=57 dynes, Pxmax=160.4 mOsm/kg; CPP<1. The pattern suggested a possibility of carcinoma. X-rays, CT scan, and liver function tests did not show any evidence of metastasis. Bronchoscopy biopsy revealed a small cell carcinoma. Suitable therapy, including was immediately initiated.

### Patient X

A 23 year old woman was tested using cell scanning technologies described herein at a check-up 2 months after being diagnosed with grand mal epilepsy. The results were within normal limits (as they were on a prior visit), except for the Pk0 which had shifted approx. 10 mOsm/kg to 136 mOsm/kg. Such a change may be attributed to her prescribed medication, phenytoin. Her treatment could be changed to carbamazepine, and her subsequent follow-up cell scans were expected to be normal.

### Patient W

A 55 year old woman presented with lethargy, fatigue, petechiae and rectal bleeding. After performing a medical history and routine physical, no apparent cause could be determined. The patient was then tested using the cell scanning technologies described herein, which revealed abnormal results namely: Cp=3.7 mL/m²; Pk0=140 mOsm/kg; IsoV=96 fL; SphV=164 fL; Inc %=71%; W10=24 mOsm/kg; Pxmin=113 mOsm/kg; Pymax=10 (fL·10⁻¹)/mOsm/kg; Pymin=−10 (fL·10⁻¹)/mOsm/kg; Py ratio=1.0; sSI=14.4; slope_{FFC}=0.4 (fL·10⁻¹)/(mOsm/kg)²; ∂ dynes=48 dynes, Pxmax=161 mOsm/kg; CPP 0. In particular, W10, Pymin, Py ratio, ∂ dynes, and CPP were greater than 3 SDs from the mean, strongly indicating that the patient was not healthy and likely to die within 2 years. When this set of parameters is greater than 3 SD from the mean, myelodysplasia is typical. Myelodysplasia was then confirmed after analysis of blood, plasma proteins and enzymes, marrow aspiration and visualization by magnetic resonance imaging and genetic analysis including karyotyping. The myelodysplasia progressed to acute myeloid leukemia. She died within 1 year of the cell scanning analysis.

### Patient V

An 8 year old boy presented with an alteration in his gait and difficulty climbing up or down stairs. He was tested using the cell scanning technologies described herein, which revealed abnormal results such as: Cp=5.2 mL/m²; Pk0=96.2 mOsm/kg; IsoV=71 fL; SphV=123 fL; Inc %=71%; W10=25 mOsm/kg; Pxmin=155 mOsm/kg; Pymax=5.7 (fL·10⁻¹)/mOsm/kg; Pymin=−28.8 (fL·10⁻¹)/mOsm/kg; Py ratio=0.2; sSI=12.4; slope_{FFC}=1.0 (fL·10⁻¹)/(mOsm/kg)²; ∂ dynes=−24 dynes, Pxmax=131.5 mOsm/kg. Given that it is atypical for patients of his age to exhibit abnormal cell scanning results, potential diagnoses were narrowed down to indications such as anorexia, leukemia, and Becker's muscular dystrophy. Becker's muscular dystrophy is the only type of muscular dystrophy to show scan abnormalities. Based on the patient's symptoms, Becker's muscular dystrophy was expected. The diagnosis was confirmed with a creatinine kinase test, electromyography, muscle biopsy, genetic testing and family history.

### Patient U

A 56 year old man was admitted to the hospital for the investigation of an anemia of unknown origin. A sample of his blood was tested using cell scanning technologies described herein and revealed grossly abnormal results such as Pk0=110 mOsm/kg and W10=27 mOsm/kg. The diagnosis of celiac disease was confirmed after antibody analysis, genetic testing for leukocyte antigens, and endoscopic jejunal biopsy. He lived for four years which was consistent with life expectancy predicted by his low Pk0.

### Example 13. Effect of HgCl₂ on RBC Membrane Permeability

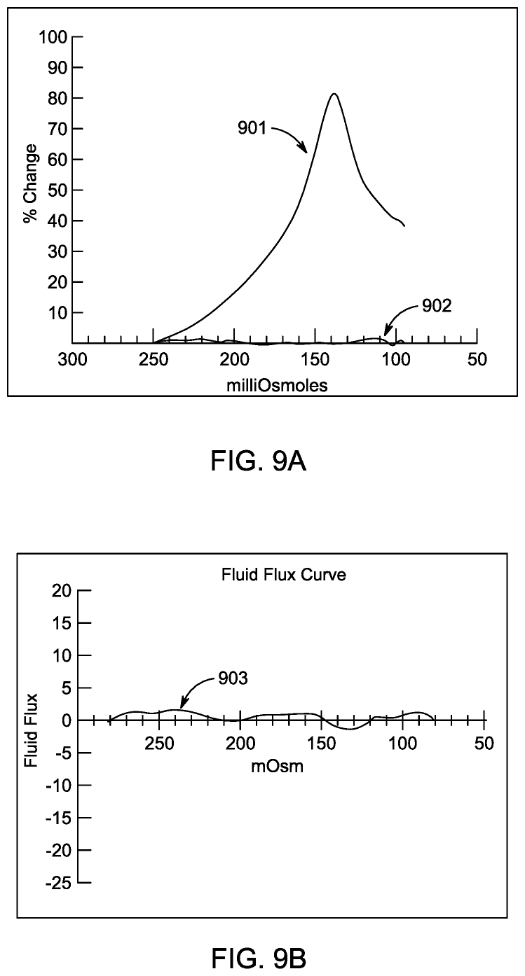

An unwashed sample of whole blood from a healthy volunteer was treated with 65 μM HgCl₂ and monitored over time using the cell scanning technologies provided herein. FIG. 10A shows a Cell Scan Plot of the sample before (901) and after (902) exposure to HgCl₂, and FIG. 10B shows a Fluid Flux Curve after (903) exposure to HgCl₂. As shown in FIG. 9A and FIG. 10B, upon addition of HgCl₂, RBC membrane permeability was essentially eliminated, and no flow of water across the membrane was detected. Upon addition of 1 mmol beta-mercaptoethanol, a known chelator of mercury(II), the effect was reversed and RBC membrane permeability was restored. Given that HgCl₂ is a known inhibitor of aquaporins (Savage, D., et al. J. Mol. Biol., 2007, 368(3):607-17), these results suggest that the cell scanning technologies provided herein may be measuring a function of aquaporins, either directly or indirectly.

### Example 14. Diagnostic Screening Technology Using Cell Scanning Technology

### Control Set—Healthy Blood Donors

A control set of blood donors was used to establish “normal” parameter values. The control set of blood donors comprised 266 directed donors and 90 volunteer donors. Fourteen parameters were evaluated and the following results were obtained. Values within 3 standard deviations of the mean were considered normal for the purposes of this experiment.

TABLE 12

| Variable | Mean −3SD | Mean | Mean +3SD |
|---|---|---|---|
| Cp (mL/m²) | 3 | 4 | 5 |
| Pk0 (mOsm/kg) | 133 | 148 | 163 |
| IsoV (fL) | 75 | 91 | 106 |
| SphV (fL) | 135 | 169 | 202 |
| Inc % (%) | 60 | 85 | 108 |
| W10 (mOsm/kg) | 15 | 19 | 22 |
| Pxmin (mOsm/kg) | 111 | 130 | 150 |
| Pxmax (mOsm/kg) | 148 | 165 | 180 |
| Pymax ((fL · 10⁻¹/mOsm/kg) | 9.6 | 12.9 | 16.4 |
| Pymin ((fL · 10⁻¹/mOsm/kg) | 11.6 | 19.6 | 27.6 |
| Py ratio | 0.4 | 0.7 | 0.9 |
| sSI | 14 | 15.7 | 17.3 |
| slope_{FFC} ((fL · 10⁻¹/mOsm/kg)²) | −1.6 | 0.7 | 3.1 |
| ∂ dynes (dynes) | 25 | 35 | 44 |

### Test Set

A test set of 4,280 blood samples from patients in several general hospitals with a typical distribution of illnesses, 363 of which were diagnosed with a malignancy by other methods, was compiled for statistical analysis. The test set was tested blindly using provided cell scanning technologies and compared to the control set. A binary classification was used to mark samples from the test set as “normal” or “abnormal.” If any sample fell more than three standard deviations from the mean for one or more parameters, the sample was considered abnormal. Results of this analysis are shown in Table 13 and demonstrate that provided cell scanning technologies successfully differentiate samples from healthy and unhealthy individuals.

TABLE 13

| N | Sensitivity | Specificity |
|---|---|---|
| 363 | 64.2% | 93.5% |

Patient profiles were also analyzed using a combined profile probability (CPP), generated from the mean squared sum of the normalized deviations of the measured value from the population mean for each of the fourteen parameters shown above in Table 13. CPP is calculated as follows: for each parameter, subtract the measured output value from the population mean; divide by the population SD, that value is squared; and then the fourteen values are added together. Results of this analysis are shown in Table 14 and demonstrate that provided cell scanning technologies successfully differentiate samples from healthy and unhealthy individuals.

TABLE 14

| CPP cutoff | Sensitivity | Specificity |
|---|---|---|
| 5.8 | 75.5% | 92.1% |
| 6.5 | 67.8% | 94.4% |

### APPENDIX A: CERTAIN ASPECTS OF WO 97/24598

The WO 97/24598 disclosure provides a new method in which a sample of cells suspended in a liquid medium, wherein the cells have at least one measurable property distinct from that of the liquid medium, is subjected to analysis to determine a measure of cell permeability of the sample of cells by a method including the steps:
(a) passing a first aliquot of the sample cell suspension through a sensor,
(b) measuring said at least one property of the cell suspension,
(c) recording the measurement of said property for the first aliquot of cells,
(d) subjecting a second aliquot of the sample cell suspension to an alteration in at least one parameter of the cell environment which has the potential to induce a flow of fluid across the cell membranes and thereby alter the said at least one property of the cells,
(e) passing said second aliquot through a sensor,
(f) measuring said at least one property of the cell suspension under the altered environment,
(g) recording the measurement of said at least one property for the second aliquot of cells,
(h) comparing the data from steps (c) and (g) as a function of the extent of said alteration of said parameter of the cell environment and change in the recorded measurements of said at least one property to determine a measure of cell permeability of the sample.

Preferably, the property of the cells which differs from the liquid medium is one which is directly related to the volume of the cell. Such a property is electrical resistance or impedance which may be measured using conventional particle counters such as the commercially available instrument sold under the trade name Coulter Counter by Coulter Instruments Inc. Preferably, the sensor used to detect cells and measure a change in the cells' property is that described in WO 97/24600. In this apparatus the cell suspension is caused to flow through an aperture where it distorts an electrical field. The response of the electrical field to the passage of the cells is recorded as a series of voltage pulses, the amplitude of each pulse being proportional to cell size.

In the preferred method of the WO 97/24598 disclosure, a measurement of cell permeability is determined by obtaining a measure of the volume of fluid which crosses a sample cell membrane in response to an altered environment. The environmental parameter which is changed in the method may be any change which results in a measurable property of the cells being altered. Preferably, a lytic agent is used to drive fluid across the cell membranes and thereby cause a change in cell volume. Preferably therefore, the environmental parameter change is an alteration in osmolality, most preferably a reduction in osmolality. Typically, the environment of the first aliquot is isotonic and thus the environment of the second aliquot is rendered hypotonic. Other suitable lytic agents include soap, alcohols, poisons, salts, and an applied shear stress.

It is possible to subject only a single aliquot of sample suspension to one or more alterations in osmolality to achieve this effect, although is preferred to use two or more different aliquots of the same sample suspension. Most preferably, the sample suspension is subjected to a continuous osmotic gradient, and in particular an osmotic gradient generated in accordance with the method of WO 97/24599.

In the preferred method of WO 97/24601, a number of measurements of particular cell parameters are made over a continuous series of osmolalities, including cell volume and cell surface area, which takes account of the deviation of the cells from spherical shape particles commonly used to calibrate the instruments. An estimate of in vivo cell shape made so that an accurate measurement of cell volume and cell surface area at all shapes is obtained. A sample suspension is fed continuously into a solution the osmolality of which is changed continuously to produce a continuous concentration gradient. Reducing the osmolality of the solution surrounding a red blood cell below a critical level causes the cell first to swell, then rupture, forming a ghost cell which slowly releases its contents, almost entirely hemoglobin, into the surrounding medium. The surface area of the each cell remains virtually unchanged on an increase in cell volume due to a reduction in osmolality of the cell's environment as the cell membrane is substantially inelastic. The time between initiation of the alteration of the environment in each aliquot to the passage of the cells through the sensing zone is kept constant so that time is not a factor in any calculation in cell permeability. An effect of feeding the sample under test into a continuously changing osmolality gradient, is to obtain measurements which are equivalent to treating one particular cell sample with that continuously changing gradient.

Preferably, the measurements are recorded on a cell-by-cell basis in accordance with the method of WO 97/24601. The number of blood cells within each aliquot which are counted is typically at least 1000 and the cell-by-cell data is then used to produce an exact frequency distribution of cell permeability. Suitably this density can be displayed more visibly by using different colors to give a three dimensional effect, similar to that seen in radar rainfall pictures used in weather forecasting. Alternatively, for a single solution of any tonicity, the measured parameter change could be displayed against a number of individual cells showing the same change. In this way a distribution of cell permeability in a tonicity of given osmolality can be obtained.

As discussed above, the methods in WO 97/24601 can provide an accurate estimate of cell volume, or other cell parameter related to cell volume, and cell surface area over a continuous osmotic gradient for individual cells in a sample. A plot of change in cell volume against osmolality reveals a characteristic curve showing how the cell volume changes with decreasing osmolality and indicates maximum and minimum rates of flow across the membrane and the flow rates attributed to a particular or series of osmotic pressures.

Having obtained measures of osmotic pressure (Pₒₛₘ), cell volume, surface area (SA) and other relevant environmental factors, it is possible to obtain a number of measures of cell permeability:

### 1) Cp Rate

This coefficient of permeability measures the rate of fluid flow across a square meter of membrane in response to a specified pressure. All positive rates represent a net flow into the cell, while all negative rates are the equivalent of a net flow out of the cell. The rate is determined by:

Cp rate=Δcell volume/ΔPₒₛₘ/SA at S.T.P.

### 2) Permeability CONSTANT pkₙ

This set of permeability measures describe each pressure where the net permeability rate is zero, and are numbered pk₀, pk₁ . . . pkₙ.

(i) pk₀ coincides with the minimum absolute pressure (hypotonic) to which a cell can be subjected without loss of integrity. A pressure change of one tenth of a milliosmole per kg (0.0001 atms) at pk₀ produces a change in permeability of between one and two orders of magnitude making pk₀ a distinct, highly reproducible measure.

(ii) pk₁ is a measure of the cells' ability to volumetrically regulate in slightly hypotonic pressures. After a certain pressure, the cell can no longer defeat the osmotic force, resulting in a change in the cell's volume. pk₁ provides a measure of the cells ability to perform this regulation, thereby measuring a cell's maximum pump transfer capability.

(iii) pk₂, a corollary of pk₁ is a measure of the cells ability to volumetrically regulate in hypertonic pressures, and occurs at low differential pressures, when compared to the cell's typical in vivo hydrostatic pressure.

The permeability constant pkₙ is described by the following equation:

pkₙ=ΔPₒₛₘ/SA at S.T.P.

When calculating pk₀, ΔPₒₛₘ=(isotonic pressure)−(pressure where net flow is zero); when calculating pk₁, ΔPₒₛₘ=(isotonic pressure)−(first hypotonic pressure where net positive flow begins). The calculation of pk₂ is identical to pk₁ except ΔPₒₛₘ measures the first hypertonic pressure where net positive flow is not zero.

### 3) CPA

This dimensionless value is the comparison of any two Cp rates, and is expressed as the net amount of fluid to cross the cell membrane between any two lytic concentrations. It provides a volume independent and pressure dependent comparison of permeability rates. This measure may be used to compare permeability changes in the same individual over a period ranging from minutes to months.

### 4) Cpₘₐₓ

This is the maximum rate of flow across the cell's membrane. For almost all cells, there are two maxima, one positive (net flow into the cell) and one negative (net flow out of the cell) situated either side of pk₀. Cpₘₐₓ is determined by detecting the maximum positive and negative gradients of the continuous curve of change in cell volume against osmolality.

### 5) Membrane Structural Resistance (MSR)

This is a measure of the structural forces inside a cell which resist the in-flow or out-flow of water. It is determined by the ratio of Cpₘₐₓ to all other non-zero flow rates into the cell. As the membrane is theoretically equally permeable at all pressures, change from the maximum flow rate outside the pressure range of pk₁ to pk₂ are due to mechanical forces. It is clear that pk₀ is an entirely mechanical limit on the cell because as Cpᵣₐₜₑ approaches zero, MSR approaches co, thereby producing more strain than the membrane can tolerate.

MSR=Cpₘₐₓ/Cpᵣₐₜₑ×100%

### 6) Cpml

This is a measure of the physiological permeability available to an individual per unit volume of tissue or blood, or for the whole organ or total body, and is calculated by:

Cpml=Δ cell volume/ΔPₒₛₘ/m³ per ml of whole blood

### 7) Cpₙₑₜ

Cpₙₑₜ is defined as the rate at which fluid can be forced across a unit area of membrane at standard temperature and pressure over unit time and is a pressure independent measure of the coefficient of permeability, given by the equation:

$${CP}_{net} = \frac{( {Volume}_{sph} - {Volume}_{iso} )}{SA}$$

FIG. 10 shows schematically the arrangement of a blood sampler for use in the method of the WO 97/24598 disclosure. The blood sampler comprises a sample preparation section 1, a gradient generator section 2 and a sensor section 3.

A whole blood sample 4 contained in a sample container 5 acts as a sample reservoir for a sample probe 6. The sample probe 6 is connected along PTFE fluid line 26 to a diluter pump 7 via multi-position distribution valve 8 and multi-position distribution valve 9. The diluter pump 7 draws saline solution from a reservoir (not shown) via port #1 of the multi-position distribution valve 9. As will be explained in detail below, the diluter pump 7 is controlled to discharge a sample of blood together with a volume of saline into a first well 10 as part of a first dilution step in the sampling process.

In a second dilution step, the diluter pump 7 draws a dilute sample of blood from the first well 10 via multi-position distribution valve 11 into PTFE fluid line 12 and discharges this sample together with an additional volume of saline into a second well 13. The second well 13 provides the dilute sample source for the gradient generator section 2 described in detail below.

Instead of using whole blood, a pre-diluted sample of blood 14 in a sample container 15 may be used. In this case, a sample probe 16 is connected along PTFE fluid line 30, multi-position distribution valve 11, PTFE fluid line 12 and multi-position distribution value 9 to the diluter pump 7. In a second dilution step, the diluter pump 7 draws a volume of the pre-diluted sample 14 from the sample container 15 via fluid line 30 and multi-position distribution value 11 into fluid line 12 and discharges the sample together with an additional volume of saline into the second well 13 to provide the dilute sample source for the gradient generator section 2.

The gradient generator section 2 comprises a first fluid delivery syringe 17 which draws water from a supply via multi-position distribution valve 18 and discharges water to a mixing chamber 19 along PTFE fluid line 20. The gradient generator section 2 also comprises a second fluid delivery syringe 21 which draws the diluted sample of blood from the second well 13 in the sample preparation section 1 via multi-position distribution valve 22 and discharges this to the mixing chamber 19 along PTFE fluid line 23 where it is mixed with the water from the first fluid delivery syringe 17. As will be explained in detail below, the rate of discharge of water from the first fluid delivery syringe 17 and the rate of discharge of dilute blood sample from the second fluid delivery syringe 21 to the mixing chamber is controlled to produce a predetermined concentration profile of the sample suspension which exits the mixing chamber 19 along PTFE fluid line 24. Fluid line 24 is typically up to 3 metres long. A suitable gradient generator is described in detail in the Applicant's WO 97/24529.

As will also be explained in detail below, the sample suspension exits the mixing chamber 19 along fluid line 24 and enters the sensor section 3 where it passes a sensing zone 25 which detects individual cells of the sample suspension before the sample is disposed of via a number of waste outlets.

In a routine test, the entire system is first flushed and primed with saline, as appropriate, to clean the instrument, remove pockets of air and debris, and reduce carry-over.

The diluter pump 7 comprises a fluid delivery syringe driven by a stepper motor (not shown) and is typically arranged initially to draw 5 to 10 ml of saline from a saline reservoir (not shown) via port #1 of multi-position distribution valve 9 into the syringe body. A suitable fluid delivery syringe and stepper motor arrangement is described in detail in the Applicant's WO 97/24599. Port #1 of the multi-position distribution valve 9 is then closed and port #0 of both multi-position distribution valve 9 and multi-position distribution valve 8 are opened. Typically 100 μl of whole blood is then drawn from the sample container 5 to take up the dead space in the fluid line 26. Port #0 of multi-position distribution valve 8 is then closed and any blood from the whole blood sample 4 which has been drawn into a fluid line 27 is discharged by the diluter pump 7 to waste via port #1 of multi-position distribution valve 8.

In a first dilution step, port #0 of multi-position distribution value 8 is opened and the diluter pump 7 draws a known volume of whole blood, typically 1 to 20 μl, into PTFE fluid line 27. Port #0 is then closed, port #2 opened and the diluter pump 7 discharges the blood sample in fluid line 27 together with a known volume of saline in fluid line 27, typically 0.1 to 2 ml, into the first well 10. Port #2 of multi-position distribution value 8 and port #0 of multi-position distribution value 9 are then closed.

Following this, port #0 of multi-position distribution valve 11 and port #3 of multi-position distribution valve 9 are opened to allow the diluter pump 7 to draw the first sample dilution held in the first well 10 to take up the dead space in PTFE fluid line 28. Port #0 of multi-position distribution valve 11 is then closed and port #1 opened to allow the diluter pump 7 to discharge any of the first sample dilution which has been drawn into fluid line 12 to waste via port #1.

In a second dilution step, port #0 of multi-position distribution valve 11 is re-opened and the diluter pump 7 draws a known volume, typically 1 to 20 μl, of the first sample dilution into fluid line 12. Fluid line 12 includes a delay coil 29 which provides a reservoir to prevent the sample contaminating the diluter pump 7. Port #0 of multi-position distribution valve 11 is then closed, port #3 opened, and the diluter pump 7 then discharges the first sample dilution in fluid line 12, together with a known volume of saline, typically 0.1 to 20 ml, into the second well 13. Port #3 of multi-position distribution valve 11 is then closed. At this stage, the whole blood sample has been diluted by a ratio of typically 10000:1. As will be explained below, the instrument is arranged automatically to control the second dilution step to vary the dilution of the sample suspension to achieve a predetermined cell count to within a predetermined tolerance at the start of a test routine.

In the gradient generator section 2, the first fluid delivery syringe 17 is primed with water from a water reservoir. Port #3 of multi-position distribution valve 22 is opened and the second fluid delivery syringe draws a volume of the dilute blood sample from the second well 13 into the syringe body. Port #3 of multi-position distribution valve 22 is then closed and port #2 of both multi-position distribution valve 18 and multi-position distribution valve 22 are opened prior to the controlled discharge of water and dilute blood sample simultaneously into the mixing chamber 19.

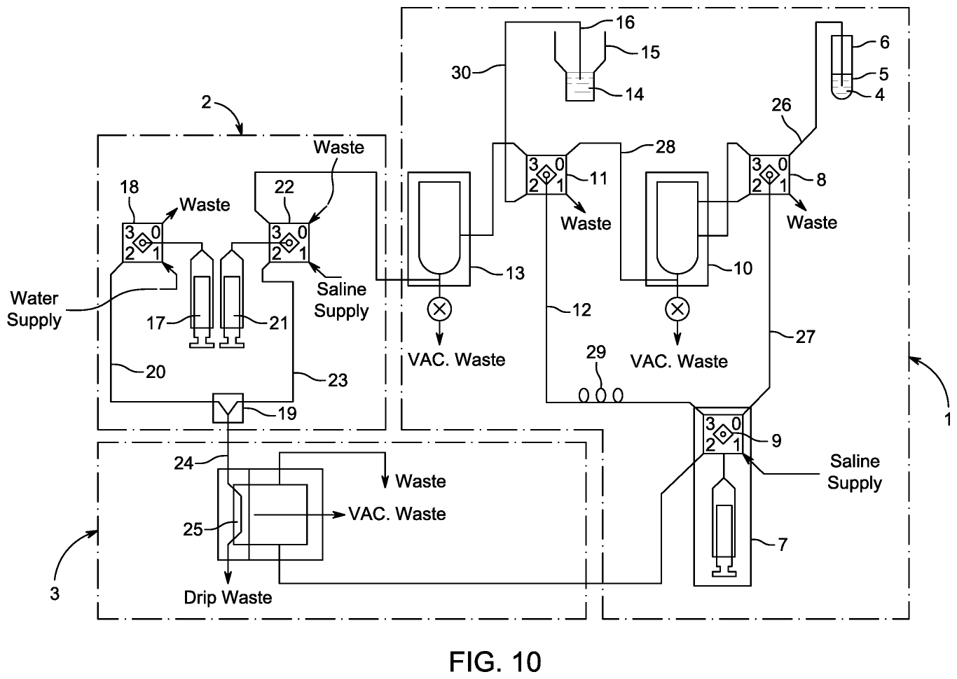

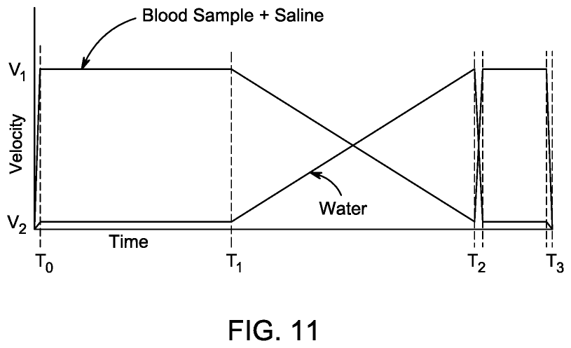

FIG. 11 shows how the velocity of the fluid discharged from each of the first and second fluid delivery syringes is varied with time to achieve a predetermined continuous gradient of osmolality of the sample suspension exiting the mixing chamber 19 along fluid line 24. The flow rate of the sample suspension is typically in the region of 200 μl s⁻¹ which is maintained constant whilst measurements are being made. This feature is described in detail in the Applicant's WO 97/24529. As shown in FIG. 2, a cam profile associated with a cam which drives fluid delivery syringe 21 accelerates the syringe plunger to discharge the sample at a velocity V₁, whilst a cam profile associated with a cam which drives fluid delivery syringe 17 accelerates the associated syringe plunger to discharge fluid at a lower velocity V₂. Once a constant flow rate from each delivery syringe has been established at time T₀, at time T₁ the cam profile associated with fluid delivery syringe 21 causes the rate of sample discharge to decelerate linearly over the period T₂−T₁, to a velocity V₂, while simultaneously, the cam profile associated with fluid delivery syringe 17 causes the rate of fluid discharge to accelerate linearly to velocity V₁. During this period, the combined flow rate of the two syringes remains substantially constant at around 200 μl s⁻¹. Finally, the two syringes are flushed over the period T₃−T₂.

Once both the first fluid delivery syringe 17 and the second fluid delivery syringe 21 have discharged their contents, the first delivery syringe is refilled with water in preparation for the next test. If a blood sample from a different subject is to be used, the second fluid delivery syringe 21 is flushed with saline from a saline supply via port #1 of multi-position distribution valve 22 to clean the contaminated body of the syringe.

The sample suspension which exits the mixing chamber 19 passes along fluid line 24 to the sensor section 3. A suitable sensor section is described in detail in the Applicant's WO 97/24600. The sample suspension passes to a sensing zone 25 comprising an electrical field generated adjacent an aperture through which the individual cells of the sample suspension must pass. As individual blood cells of the sample suspension pass through the aperture the response of the electrical field to the electrical resistance of each individual cell is recorded as a voltage pulse. The amplitude of each voltage pulse together with the total number of voltage pulses for a particular interrupt period, typically 0.2 seconds, is also recorded and stored for subsequent analysis including a comparison with the osmolality of the sample suspension at that instant which is measured simultaneously. The osmolality of the sample suspension may also be determined without measurement from a knowledge of the predetermined continuous osmotic gradient generated by the gradient generator section 2. As described below, the osmolality (pressure) is not required to determine the cell parameters.

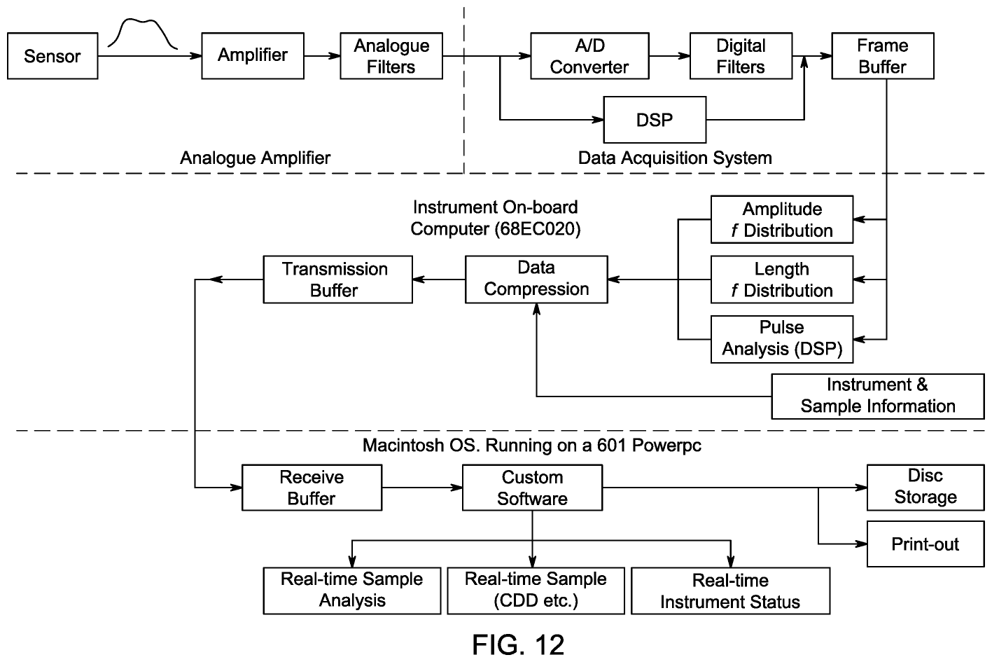

FIG. 12 shows how data is collected and processed. Inside each instrument is a main microprocessor which is responsible for supervising and controlling the instrument, with dedicated hardware or low-cost embedded controllers responsible for specific jobs within the instrument, such as operating diluters, valves, and stepper motors or digitizing and transferring a pulse to buffer memory. The software which runs the instrument is written in C and assembly code and is slightly less than 32 K long.

When a sample is being tested, the amplitude and length of each voltage pulse produced by the sensor is digitized to 12-bit precision and stored in one of two 16K buffers, along with the sum of the amplitudes, the sum of the lengths, and the number of pulses tested. Whilst the instrument is collecting data for the sensors, one buffer is filled with the digitized values while the main microprocessor empties and processes the full buffer. This processing consists of filtering out unwanted pulses, analyzing the data to alter the control of the instrument and finally compressing the data before it is sent to the personal computer for complex analysis.

Optional processing performed by the instrument includes digital signal processing of each sensor pulse so as to improve filtering, improve the accuracy of the peak detection and to provide more information about the shape and size of the pulses. Such digital signal processing produces about 25 16-bit values per cell, generating about 25 megabytes of data per test.

Data processing in the personal computer consists of a custom 400K program written in C and Pascal. The PC displays and analyses the data in real time, controls the user interface (windows, menus, etc.) and stores and prints each sample.

The software also maintains a database of every sample tested enabling rapid comparison of any sample which has been previously tested. Additionally, the software monitors the instrument's operation to detect malfunctions and errors, such as low fluid levels, system crashes or the user forgetting to turn the instrument on.

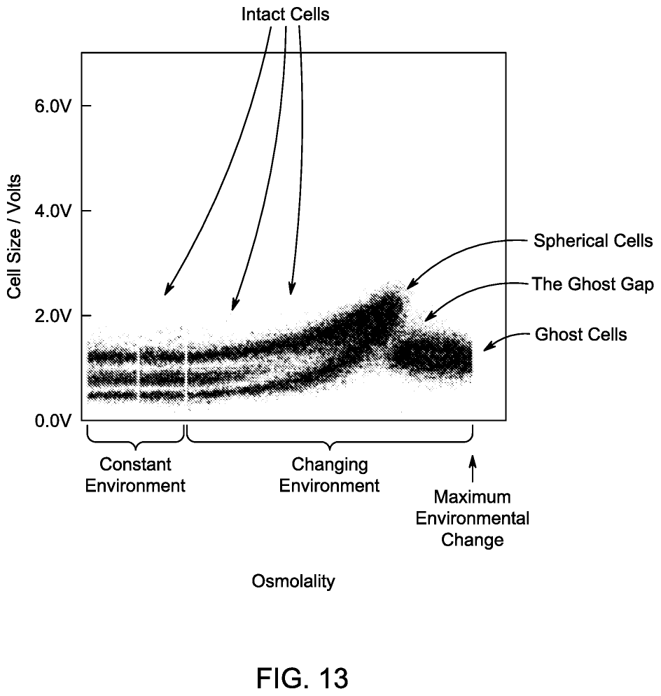

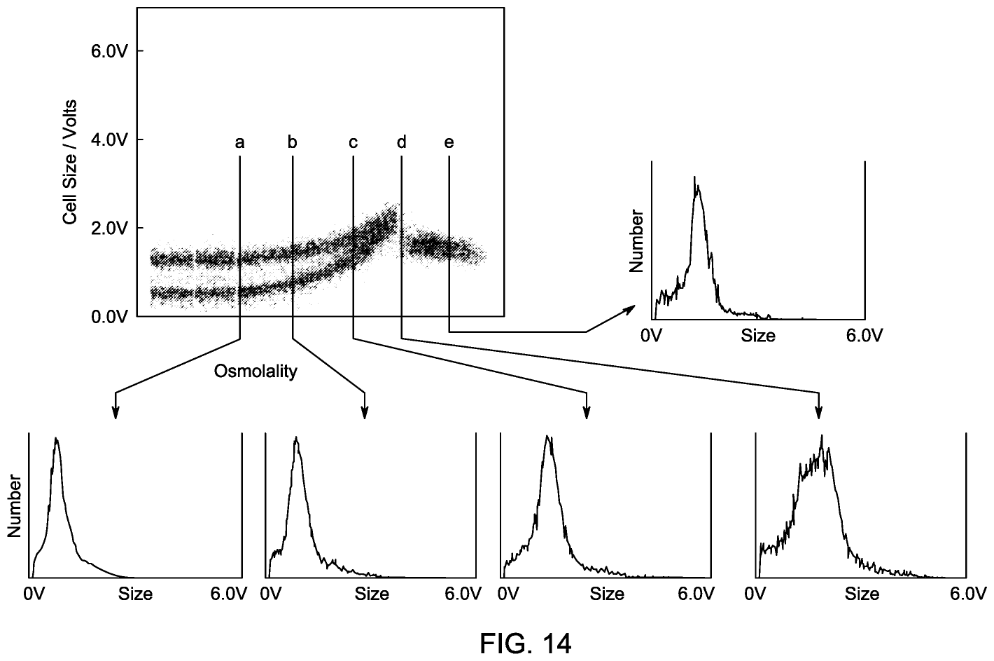

The voltage pulse generated by each cell of the sample suspension as it passes through the aperture of sensing zone 25 is displayed in graphical form on a VDU of a PC as a plot of osmolality against measured voltage. The sample suspension passes through the sensor section at a rate of 200 μl s⁻¹. The second dilution step is controlled to achieve an initial cell count of around 5000 cells per second, measured at the start of any test, so that in an interrupt period of 0.20 seconds, around 1000 cells are detected and measured. This is achieved by varying automatically the volume of saline discharged by the diluter pump 7 from the fluid line 12 in the second dilution step. Over a test period of 40 seconds, a total of 200 interrupt periods occur and this can be displayed as a continuous curve in a three-dimensional form to illustrate the frequency distribution of measured voltage at any particular osmolality, an example of which is shown in FIG. 13 and FIG. 14.

The measured cell voltage, stored and retrieved on an individual cell basis is shown displayed on a plot of voltage against the osmolality of the solution causing that voltage change. Using individual dots to display the measured parameter change for each individual cell results in a display whereby the distribution of cells by voltage, and thereby by volume, in the population is shown for the whole range of solutions covered by the osmolality gradient. The total effect is a three-dimensional display shown as a measured property change in terms of the amplitude of the measured voltage pulses against altered parameter, in this case the osmolality of the solution, to which the cells have been subjected and the distribution or density of the cells of particular sizes within the population subjected to the particular osmolality. The effect is to produce a display analogous to a contour map, which can be intensified by using colour to indicate the areas of greatest intensity.

When full data is available on the distribution of cell size in a particular population of cells subjected to hemolytic shock in a wide range of hypotonic solutions, at osmolalities just below a critical osmolality causing lysis, a gap in the populations is visible. As shown in FIG. 13, ghost cells are fully visible or identifiable in the three-dimensional plot and the unruptured cells are clearly identifiable, but between them is a region defined by osmolality and cell volume where relatively few individuals appear. The existence of this phenomenon, which we have termed the “ghost gap”, has not previously been recognized.

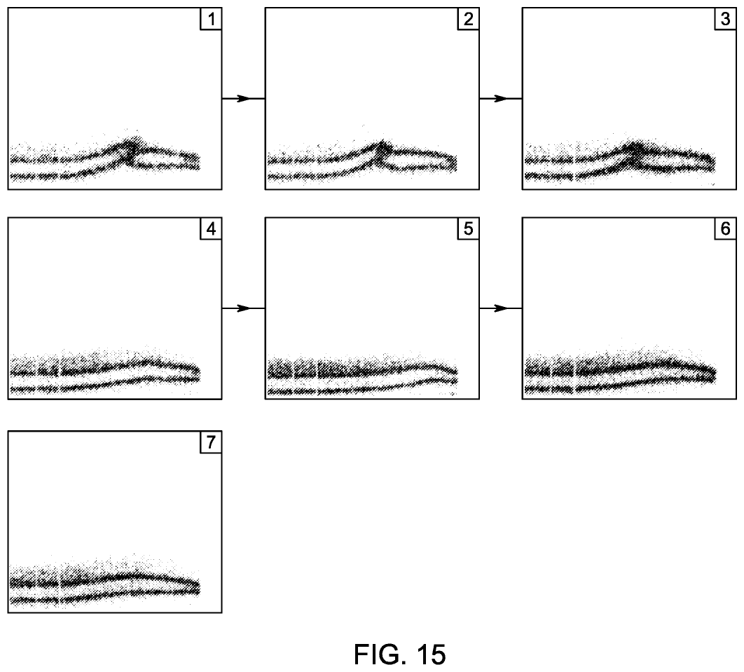

If the entire series of steps are repeated at timed intervals on further aliquots of the original sample and the resulting measured voltage is plotted against osmolality, time and frequency distribution, a four-dimensional display, is obtained which may be likened to a change in weather map. This moving three-dimensional display, its motion in time being the fourth dimension, provides an additional pattern characteristic of a particular blood sample. This is shown in the series of images in FIG. 15. The images shown in FIG. 15 are the results of tests carried out at hourly intervals at a temperature of 37° C. As the measurements are so exact, the repeat values are superimposable using computer sequencing techniques.

As shown, cells slowly lose their ability to function over time, but they also change in unexpected ways. The size and shape of the cells in a blood sample change in a complex, non-linear but repeatable way, repeating some of the characteristic patterns over the course of days and on successive testing. The patterns, emerging over time, show similarity among like samples and often show a characteristic wave motion. The pattern of change may vary between individuals reflecting the health of the individual, or the pattern may vary within a sample. Thus a sample that is homogeneous when first tested may split into two or several sub-populations which change with time and their existence can be detected by subjecting the sample to a wide range of different tonicities and recording the voltage pulse in the way described. As shown in FIG. 15, after the first few hours the cell becomes increasingly spherical in the original sample, it then becomes flatter for several hours, then more spherical again, reaches a limit, and then becomes thinner and finally may swell again. It has been determined that the rate at which observed changes take place are influenced by pH, temperature, available energy and other factors.

The three-dimensional pattern provides data which enables identification of the precise osmolality at which particular cells reach their maximum volume, when they become spheres. With appropriate calibration, which is described in detail below, and using the magnitude of the voltage pulse, it is possible to define precisely and accurately the actual volume of such cells and thereafter derive a number of other cell parameters of clinical interest.

The amplitude of the voltage pulses produced by the sensor 25 as individual cells pass through the electrical field are proportional to the volume of each cell. However, before a conversion can be performed to provide a measure of cell volume, the instrument requires calibration. This is performed using spherical latex particles of known volume and by comparison with cell volumes determined using conventional techniques.

Experimental results have shown that the mapping of measured voltage to spherical volume of commercially available latex particles is a linear function. Accordingly, only a single size of spherical latex particles needs to be used to determine the correct conversion factor. In a first calibration step, a sample containing latex particles manufactured by Bangs Laboratories Inc. having a diameter of 5.06 μm i.e. a volume of 67.834 m³, was sampled by the instrument. In this particular test, the instrument produced a mean voltage of 691.97 mV. The spherical volume is given by the equation:

Spherical volume=measured voltage×Kᵥₒₗₜₛ

where Kᵥₒₗₜₛ is the voltage conversion factor.

Re-arranging this equation gives:

$${K}_{volts} = \frac{spherical volume}{measured voltage}$$

which in this case gives,

$${K}_{volts} = \frac{67.834}{691.97} = 0.0980$$

This value of Kᵥₒₗₜₛ is only valid for the particular instrument tested and is stored in a memory within the instrument.

In a second calibration step, a shape correction factor is determined to take account of the fact that the average blood cell in the average individual has a bi-concave shape. Applying the above voltage conversion factor Kᵥₒₗₜₛ assumes that, like the latex particles, blood cells are spherical and would therefore give an incorrect cell volume for cell shapes other than spherical. In the WO 97/24598 disclosure, a variable shape correction function is determined so that the mean volume of the blood cells at any osmolality up to the critical osmolality causing lysis can be calculated extremely accurately.

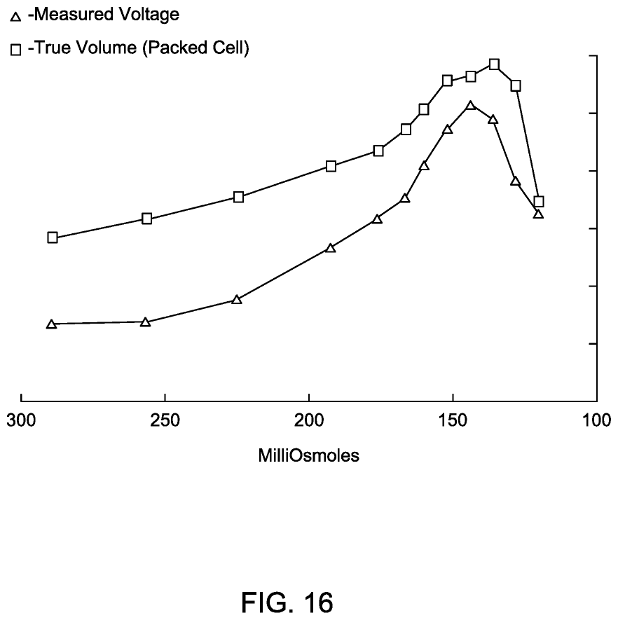

To illustrate this, a sample was tested at a number of accurately known osmolalities and the volume of the blood cells measured using a standard reference method, packed cell volume. A portion of the same sample was also tested by the method of the present invention using the instrument of FIG. 10 to measure the voltage pulses from individual cells at the corresponding osmolalities. The results of these procedures are plotted as two superimposed graphs of osmolality (x-axis) against measured voltage and true volume, respectively, in FIG. 16.

At an isotonic osmolality of 290 mOsm, the true volume, as determined by the packed cell volume technique, was 92.0 fL, whilst the measured mean voltage was 670 mV. The true isotonic volume of the cells is given by equation:

Volumeᵢₛₒ=Voltageᵢₛₒ×Kᵥₒₗₜₛ×Kₛₕₐₚₑ
where Voltageᵢₛₒ is the measured voltage and Kₛₕₐₚₑ is a shape correction factor. Re-arranging:

$${K}_{shape} = \frac{{Volume}_{iso}}{{Voltage}_{iso} \times {K}_{volts}}$$

which in this example gives,

$${K}_{shape} = \frac{92.0}{670 \times 0.0980} = 1.4$$

The shape correction factor Kₛₕₐₚₑ for each of the aliquots is different with the maximum shape correction being applied at isotonic osmolalities where the blood cells are bi-concave rather than spherical. To automate the calculation of Kₛₕₐₚₑ at any osmolality of interest a shape correction function is required. The following general function describes a shape correction factor based on any two sensor readings i.e. measured voltages:

f(Kₛₕₐₚₑ)=f(SR1,SR2)

where SR1 is a sensor reading (measured voltage) at a known shape, typically spherical, and

SR2 is a sensor reading (measured voltage) at an osmolality of interest, typically isotonic.

Analysis has shown that this is a linear function and that:

$$f ( {K}_{shape} ) = 1 + [ \frac{( SR 1 - SR 2 )}{( SR 1 )} ] \times {K}_{a}$$

where Kₐ is an apparatus dependent constant, which is determined as follows:

Kₛₕₐₚₑ at an osmolality of 290 mOsm is known (see above), applying the values SR1=1432 mV, SR2=670 mV and Kₛₕₐₚₑ=1.4 to the above equation gives:

$$1.4 = 1 + [ \frac{( 1432 - 670 )}{1432} ] \times {K}_{a}$$

rearranging:

Kₐ=0.7518

This value of Kₐ is constant for this instrument.

The true isotonic volume of a blood sample is determined by comparing the measured voltage at an isotonic volume of interest with the measured voltage of cells of the same blood sample at some known or identifiable shape, most conveniently cells which have adopted a spherical shape, whereby:

$${Volume}_{iso} = {Voltage}_{jso} \times {K}_{volts} \times f ( {K}_{shape} ) = SR 2 \times 0.0980 \times [ 1 + [ \frac{( SR 1 - SR 2 )}{( SR 1 )} ] \times 0.7518 ]$$

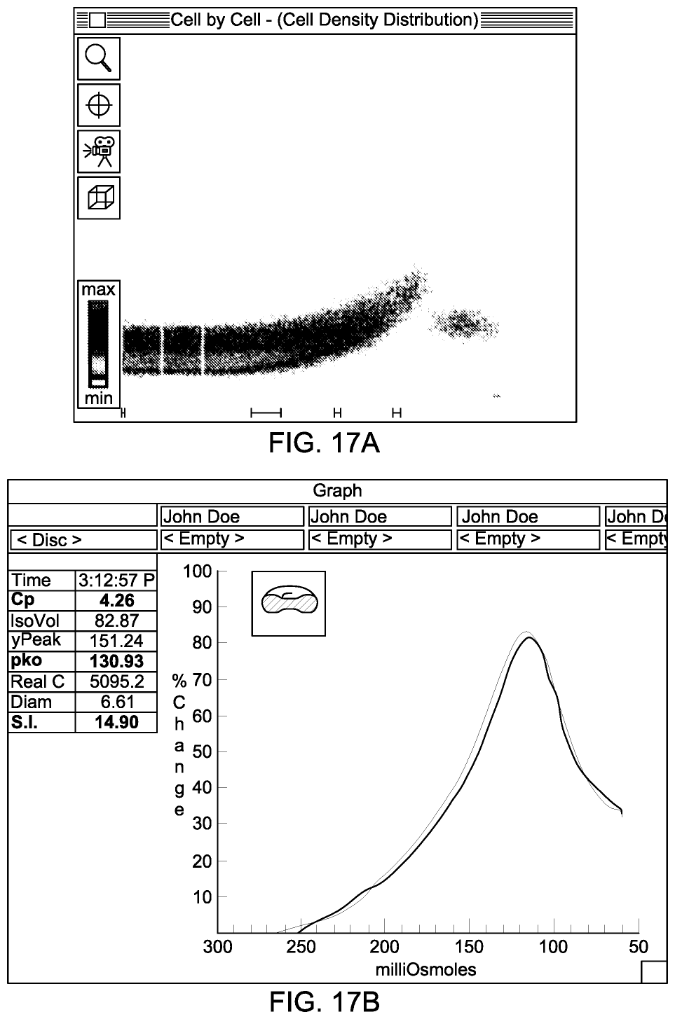

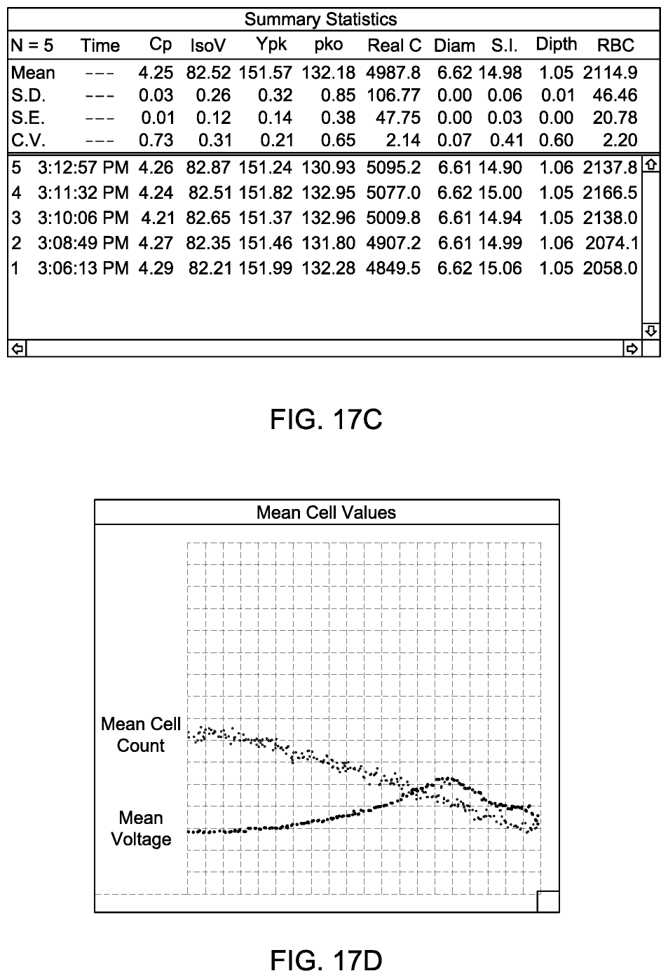

In the WO 97/24598 disclosure, the point at which the blood cells become spherical when subjected to a predetermined continuous osmotic gradient can be determined very accurately. FIGS. 17A-17D show the results for a blood sample. FIG. 17A shows a three-dimensional plot of measured voltage against osmolality, FIG. 17B shows a graph of osmolality against percentage change in measured voltage for a series of tests of a sample, FIG. 17C shows the results in a tabulated form, and FIG. 17D shows superimposed graphs of mean voltage and cell count for the test, respectively, against osmolality. As shown, the cell count, which is initially 5000 cells per second at the beginning of a test, reduces throughout the test due to the dilution of the sample in the gradient generator section 2. The mean voltage rises to a maximum at a critical osmolality where the blood cells achieve a spherical shape and then reduces. Using standard statistical techniques, the maxima of the curve in FIG. 17B, and therefore the mean voltage at the maxima, can be determined. The mean voltage at this point gives the value SR1 for the above equation. It is then possible to select any osmolality of interest, and the associated measured voltage SR2, and calculate the true volume of the cell at that osmolality. Typically, the isotonic osmolality is chosen, corresponding to approximately 290 mOsm.

For the above test, at 290 mOsm, SR1=1432 mV and SR2=670 mV. Accordingly:

$${f ( {K}_{shape} )}_{290} = 1 + [ \frac{1432 - 670}{1432} ] \times 0.7518$$

K_{shape 290}=1.40

and therefore:

$$\begin{matrix}{Volume}_{iso} = SR 2 \times {K}_{volts} \times {K}_{shape} \\ = 670 \times 0.0980 \times 1.40 \\ = 91.92 fL ,\end{matrix}$$

and:

$$\begin{matrix}{Volume}_{sph} = SR 1 \times {K}_{volts} \times {K}_{shape} \\ = 1432 \times 0.098 \times 1.0 \\ = 140.34 fL\end{matrix}$$

Knowledge of the mean volume of the sphered cells allows calculation of spherical radius as:

$${Volume}_{sph} = \frac{4 π {r}^{3}}{3}$$

from which the spherical radius

$$r = {[ \frac{3 \times {Volume}_{sph}}{4 π} ]}^{\frac{1}{3}}$$

$$\begin{matrix}r = {[ \frac{3 \times 140.34}{4 π} ]}^{\frac{1}{3}} \\ = 3.22 μ m\end{matrix}$$

Having determined volumeᵢₛₒ, volumeₛₚₕ and the spherical cell radius, it is possible to calculate a number of other parameters. In particular:

### 1. Surface Area (SA)

Since the surface area SA is virtually unchanged at all osmolalities, the cell membrane being virtually inelastic, and in particular between spherical and isotonic, the surface area SA may be calculated by substituting r into the expression:

$$\begin{matrix}SA = 4 π {r}^{2} \\ = 4 π \times {( 3.22 )}^{2} \\ = 130.29 μ {m}^{2}\end{matrix}$$

### 2. Surface Area to Volume Ratio (SAVR)

Given that the walls of a red cell can be deformed without altering their area, once the surface area SA is known for a cell or set of cells of any particular shape, the surface area is known for any other shape, thus the surface area to volume ratio SAVR can be calculated for any volume. SAVR is given by the expression:

$$\begin{matrix}SAVR = \frac{4 π {r}^{2}}{{Volume}_{iso}} = \frac{SA}{{Volume}_{iso}} \\ = \frac{130.29}{91.99} \\ = 1.42\end{matrix}$$

### 3. Sphericity Index (SI)

The WO 97/24598 disclosure can easily measure the SAVR, a widely quoted but hitherto, rarely measured indication of cell shape. For a spherical cell, it has the value of 3/r, but since cells of the same shape but of different sizes may have different SAVR values, it is desirable to use the sphericity index SI which is a dimensionless unit independent of cell size, given by the expression:

$$\begin{matrix}SI = SAVR \times \frac{r}{3} \\ = 1.52 \\ = 1.42 \times \frac{3.22}{3}\end{matrix}$$

### 4. Cell Diameter (D)

When the normal cell is in the form of a bi-concave disc at isotonic osmolality, it is known that the ratio of the radius of a sphere to that of the bi-concave disc is 0.8155. On this basis, therefore, the diameter D of a cell in the form of a bi-concave disc is given by:

$$\begin{matrix}D = \frac{2 r}{0.8155} \\ = \frac{2 \times 3.22}{0.8155} \\ = 8.19 μ m\end{matrix}$$

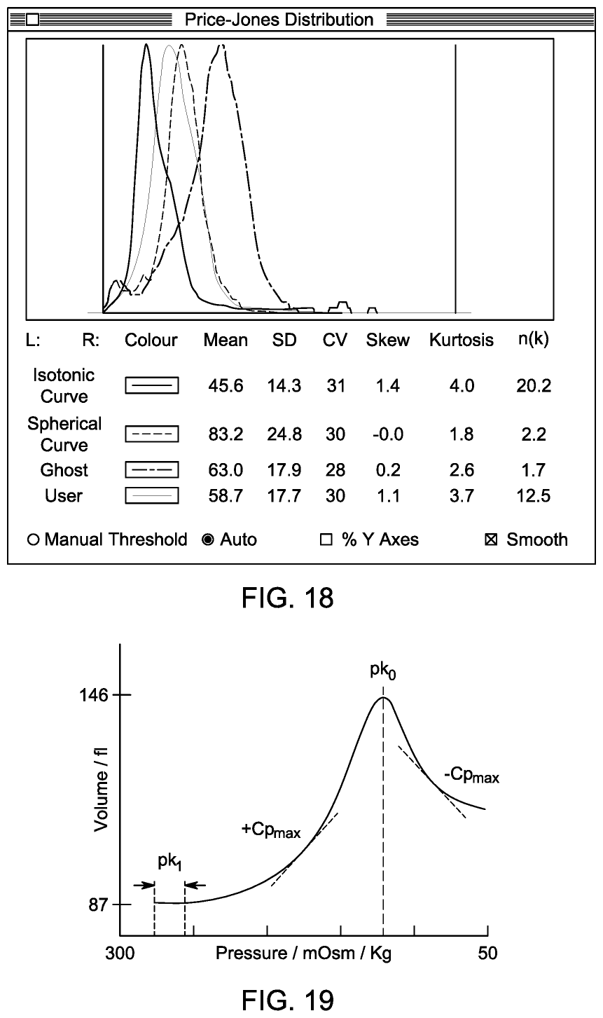

The same parameter can be determined for all other osmolalities. The frequency distribution of the cell diameters is given both as dispersion statistics as well as a frequency distribution plot. The present invention provides an automated version of the known manual procedure of plotting a frequency distribution of isotonic cell diameters known as a Price-Jones curve. The present invention is capable of producing a Price-Jones curve of cell diameters for any shape of cell and, in particular, isotonic, spherical and ghost cells (at any osmolality) and is typically based on 250,000 cells. This is shown in FIG. 18.

### 5. Cell Thickness (CT)

When the cell is in the form of a bi-concave disc, an approximate measure of the cell thickness can be derived from the cross-sectional area and the volume. The area is of course derivable from the radius of the cell in spherical form. The cell thickness can therefore be calculated as follows:

$$\begin{matrix}CT = \frac{{Volume}_{iso}}{π {r}^{2}} \\ = \frac{91.92}{π \times {3.22}^{2}} \\ = 2.82 μ m\end{matrix}$$

### 6. Surface Area Per Milliliter (SAml)

The product of the surface area (SA) and the cell count (RBC) is the surface area per milliliter (SAml) available for physiological exchange. The total surface area of the proximal renal tubes that are responsible for acid-base regulation of the body fluids is 5 m². The total surface area of the red blood cells that also play an important part in the regulation of the acid-base balance is 4572 m², almost 3 orders of magnitude larger. RBC is calculated internally from a knowledge of the flow rate of the diluted blood sample, a cell count for each sample and the dilution of the original whole blood sample. Typically, RBC is approximately 4.29×10⁹ red cells per ml.

$$\begin{matrix}SAml = SA \times RBS ( per ml ) \\ = 130.29 μ {m}^{2} \times 3.29 {10}^{9} \\ = 0.56 {m}^{2} {ml}^{- 1}\end{matrix}$$

### 7. Cell Permeability (Cp)

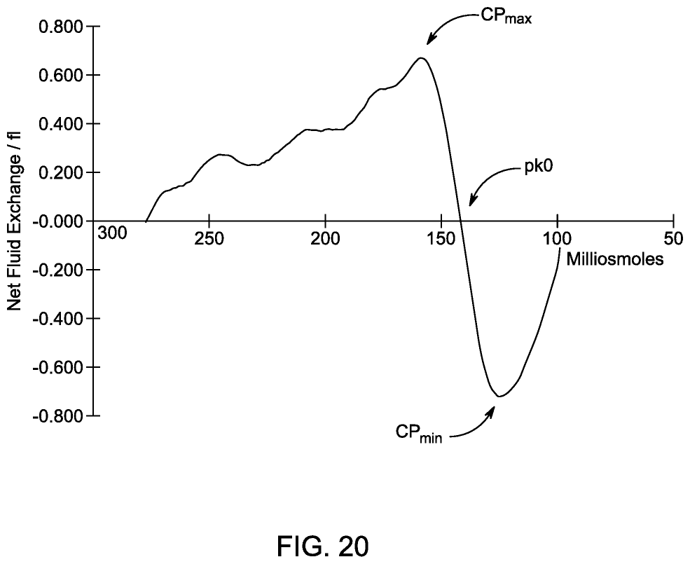

The plot of cell volume against osmolality in FIG. 19 reveals a characteristic curve showing how the cell volume changes with decreasing osmolality and indicates maximum and minimum rates of flow across the membrane and the flow rates attributed to a particular or series of osmotic pressures. Many of the cell permeability measurements are primarily dependent upon the change in volume of the cells at different pressures. The results are shown plotted as a graph of net fluid exchange against osmotic pressure in FIG. 20.

Having obtained measures of osmotic pressure (Pₒₛₘ), cell volume, surface area (SA) and other relevant environmental factors, it is possible to obtain a number of measures of cell permeability, such as Cp rate, permeability constant, CpΔ, Cpₘₐₓ, MSR, Cpml, and Cpₙₑₜ, as described above.

### APPENDIX B: CERTAIN ASPECTS OF WO 97/24601

The WO 97/24601 disclosure provides a new method in which a sample of cells suspended in a liquid medium, wherein the cells have at least one measurable property distinct from that of the liquid medium, is subjected to analysis by a method including the steps of:
(a) passing a first aliquot of the sample cell suspension through a sensor,
(b) measuring said at least one property of the cell suspension,
(c) recording the measurement of said property for the first aliquot of cells,
(d) subjecting the first or at least one other aliquot of the sample cell suspension to an alteration in at least one parameter of the cell environment which has the potential to alter the shape of the cells to a known or identifiable extent to create an altered cell suspension,
(e) passing said altered cell suspension through a sensor,
(f) measuring said at least one property of the altered cell suspension,
(g) recording the measurement of said at least one property for said altered suspension,
(h) comparing the data from steps (c) and (g) and determining a shape compensation factor to be applied to the measurement of said at least one property of the first aliquot of cells in step (c) in the calculation of a cell parameter to take account of a variation in shape between the first aliquot of cells in step (c) and said altered cell suspension in step (g).

In the WO 97/24601 disclosure, a cell parameter, for example cell volume, is determined by subjecting one or more aliquots of a sample cell suspension to one or more alterations of at least one parameter of the cell environment to identify a point at which the cells achieve a particular shape to obtain a sample specific shape compensation factor.

All existing automated methods include a fixed shape correction in the treatment of sensor readings taken from a single cell suspension in which the cell environment is not altered during the course of the test, which compensates for the deviation of the cells from spherical shape particles commonly used to calibrate the instruments. However, in a calculation of cell volume, as the cell shape is unknown, a fixed correction of approximately 1.5 is entered into the calculation on the assumption that a sample cell has the shape of a biconcave disc. This correction is correct for the average cell in the average person at isotonic osmolality, but it is incorrect for many categories of illness where the assumed fixed correction may induce an error of up to 60% in the estimate of cell volume. In the method of the WO 97/24601 disclosure, an estimate is made of the in vivo cell shape so that a true estimate of cell volume or other cell parameter at all shapes is obtained. In the preferred embodiment of the WO 97/24601 disclosure, a shape correction function is determined which is used to generate a shape correction factor which is a measure of the shape of the cell specific for that cell sample. The value of the shape correction factor generated by this function then replaces the conventional fixed shape correction of 1.5 to obtain a true measure of cell volume and other cell parameters.

According to a second aspect of the present invention, an apparatus for testing a sample cell suspension in a liquid medium in accordance with the method of the first aspect of the present invention comprises data processing means programmed to compare data from said steps (c) and (g) to determine a shape compensation factor to be applied to the measurement of said at least one property of the first aliquot of cells in the calculation of a cell parameter to take account of a variation in shape between the first aliquot of cells and said altered cell suspension.

Preferably, the data processing means comprises the internal microprocessor of a personal computer.

Preferably, the property of the cells which differs from the liquid medium is one which is directly related to the volume of the cell. Such a property is electrical resistance or impedance, and this is measured as in the normal Coulter Counter by determining the flow of electrical current through the cell suspension as it passes through a sensing zone of the sensor. The sensing zone is usually a channel or aperture through which the cell suspension is caused to flow. Any type of sensor may be used provided that the sensor produces a signal which is proportional to the cell size. Such sensor types may depend upon voltage, current, RF, NMR, optical, acoustic or magnetic properties. Most preferably, the sensor is substantially as described in WO 97/24600.

Although the method is usually carried out on blood cells, for instance white or, usually, red blood cells, it may also be used to investigate other cell suspensions, which may be plant or animal cells or micro-organism cells, for instance, bacterial cells.

The environmental parameter which is changed in the method may be any change which will result in a measurable parameter of the cells being altered. The method is of most value where the change in environmental parameter changes the size, shape, or other anatomical property of the cell. The method is of particular value in detecting a change in the volume of cells as a result of a change of osmolality of the surrounding medium. Preferably therefore, the environmental parameter change is an alteration, usually a reduction, in osmolality. Typically the environment of the first aliquot is isotonic, and thus the environment of the altered suspension in step (g) is rendered hypotonic, for instance by diluting a portion of isotonic sample suspension with a hypotonic diluent.

The method of the present invention, as well as being applicable to cells, as described above, may also be applicable to other natural and synthetic vesicles which comprise a membrane surrounding an interior space, the shape or size or deformability of which may be altered by altering an environmental parameter. Such vesicles may be useful as membrane models, for instance, or as drug delivery devices or as devices for storing and/or stabilizing other active ingredients or to contain hemoglobin in blood substitutes.

In the method, the time between the initiation of the alteration of the environment to the passage of the cells through the sensing zone may vary but preferably is less than 1 minute, more preferably less than 10 seconds. The time is generally controlled in the method and preferably it is kept constant. If it changes, then time may be a further factor which is taken into account in the calculation step of step (h).

Although it is possible for the method of the WO 97/24601 disclosure to comprise merely of the treatment of two aliquots of the sample cell suspension, more usually the method includes the steps of subjecting another aliquot of sample cell suspension to a second alteration in at least one parameter of the cell environment passing said altered aliquot through the sensor, recording the change in said property of the cell suspension under the altered environment as each of a number of cells of the aliquot passes through the sensor, recording all the concomitant properties of the environment together with the said change on a cell-by-cell basis, and comparing the data from previous step (c) and the preceding step as a function of the extent of said second alteration of environmental parameter. Usually there are many further aliquots treated in a similar way. The greater the number of aliquots tested, the greater the potential accuracy, precision and resolution of the results which are obtained. It is also possible to subject a only single aliquot of sample suspension to a series of such alterations in at least one parameter of the cell environment.

In its simplest form, the test is dependent upon two sensor measurements, one of which is at a maximum, or near to it. However, the environment required to induce a cell to reach a maximum size can be entirely unknown.

Furthermore, the environmental changes can be sequential, non-sequential, non-sequential, random, continuous or discontinuous, provided that the maximum achievable cell size is recorded. One convenient way of ensuring this is to test the cell in a continuously changing environment so that all possible cell sizes are recorded, including the maximum.

The second alteration in the cell environment is usually of the same type as the first alteration. It may even be of the same extent as the first alteration, but the time between initiation of the alteration and passage of the cells through the sensing zone may be different, thereby monitoring the rate of change in the cells properties when subjected to a particular change in environmental parameter. This technique may also be used to monitor cells which have been in storage for several years.

In another embodiment the second alteration in environmental parameter is of the same type as the first alteration, but has a different extent. In such a case, it is preferred for the time between initiation of the alteration and passage of the cells through the sensing zone to be the same for each aliquot of the cell suspension. Preferably, in this embodiment of the method second and subsequent aliquots of cell suspension are subjected to successively increasing extents of alteration of the environmental parameter such that the change of said property produces a maximum and then decreases as the extent of alteration of environmental parameter is increased. In the preferred embodiment in which the property of the cell suspension which is monitored is directly related to the volume of the cells, and where the alteration of environmental parameter for the second and subsequent aliquots results in a volume increase of the cells, preferably, the environmental change is varied until the cell volume passes a maximum.

Since the preferred application of the method of the WO 97/24601 disclosure is to analyze red blood cells, the following discussion is based mainly on the study of such cells. It will be realized, however, that the method is, as mentioned above, applicable to other cell types and to determine other information concerning an organism from a study of such cell types.

In current practice, cell shape, particularly red blood cell shape, is not estimated by any automated method. The present WO 97/24601 disclosure enables the user to determine cell shape and derive other data, such as cell volume, surface area, surface area to volume ratio, sphericity index, cell thickness, and surface area per milliliter. Aside from research and experimental laboratories, none of these measurements are currently available in any clinical laboratory and hitherto, none could be completed within 60 seconds. In particular, the preferred method where the sample cell suspension is subjected to a concentration gradient, enables the automatic detection or a user to detect accurately when the cells adopt a substantially spherical shape immediately before lysis.

The commercially available Coulter Counter particle counter instrument produces a signal in proportion to the volume of particles which pass through a sensing zone, typically a voltage pulse for each particle. The size of the signal is calibrated against spherical latex particles of known volume to produce a conversion factor to convert a measured signal, typically voltage, into a particle volume, typically femtoliters. When using particle counters of this type to measure the size of particles that are not spheres, as is typical in biological samples such as platelets, fibroblasts or red blood cells which have the shape of a disc, a fixed shape correction factor is used in addition to the conversion factor. This fixed shape correction, based on theoretical and empirical data, is designed to produce a correct volume estimate when measuring particles that are not spherical as the size of the voltage pulses are not solely related to cell volume. For instance, normal red blood cells produce sensor pulses which are too small by a factor of around 1.5 when measured on these instruments and therefore a fixed correction of 1.5 is entered into the calculation of cell volume to produce the correct valve.

In the preferred method of the WO 97/24601 disclosure, this fixed shape correction factor is replaced with a sample specific shape correction factor f(Kₛₕₐₚₑ) generated from a shape correction function (see Appendix A). The shape correction function is continuous for all cell shapes and ranges in value from 1.0 for spherical cells to infinity for a perfectly flat cell. The shape correction function increases the accuracy with which cell parameters which depend on anatomical measurement, such as cell volume, can be determined. Preferably, the shape correction factor a blood cell is determined by comparing the measured voltage (SR1) with the measured (SR2) voltage of cells of the same blood sample at some known or identifiable shape, most conveniently cells which have adopted a spherical shape.

The WO 97/24601 disclosure also provides a new method in which a sample of cells suspended in a liquid medium, wherein the cells have at least one measurable property distinct from that of the liquid medium, is subjected to analysis by a method including the steps of:
(a) passing a first aliquot of the sample cell suspension through a sensor,
(b) measuring said at least one property of the cell suspension as each of a number of cells of the first aliquot passes through the sensor,
(c) recording the measurement of said property for the first aliquot of cells on a cell-by-cell basis,
(d) subjecting the first or at least one other aliquot of the sample cell suspension to an alteration in at least one parameter of the cell environment which has the potential to alter the said at least one property of the cells to create an altered cell suspension,
(e) passing said altered cell suspension through a sensor,
(f) measuring said at least one property of the altered cell suspension as each of a number of cells of the altered cell suspension passes through the sensor,
(g) recording the measurement of said at least one property for the altered cell suspension on a cell-by-cell basis,
(h) comparing the data from steps (c) and (g) as a function of the extent of said alteration of said parameter of the cell environment and frequency distribution of said at least one property.

By carrying out the method of the WO 97/24601 disclosure, and in particular by recording the property change data for the cells on a cell-by-cell basis, the data can be subsequently treated so as to identify sub-populations of cells within the sample which respond differently to one another under the imposition of the environmental parameter alteration.

The WO 97/24601 disclosure provides a method for testing blood samples which enables data to be obtained on a cell-by-cell basis. By using the data on a cell-by-cell basis, it enables new parameters to be measured and to obtain information on the distribution of cells of different sizes among a population and reveal sub-populations of cells based on their anatomical and physiological properties.

A measure of reproducibility is the standard deviation of the observations made. An aspect of the WO 97/24601 disclosure is to provide improvements in which the standard deviation of the results obtained is reduced to ensure clinical utility.

The WO 97/24601 disclosure also provides an apparatus for testing a sample cell suspension in a liquid medium in accordance with the methods of the WO 97/24601 disclosure comprising data processing means programmed to compare data from said steps (c) and (g) as a function of the extent of said alteration of said parameter of the cell environment and frequency distribution of said at least one property.

Other environmental parameter changes which may be investigated include changes in pH, changes in temperature, pressure, ionophores, changes by contact with lytic agents, for instance toxins, cell membrane pore blocking agents or any combinations of these parameters. For instance, it may be useful to determine the effectiveness of lytic agents and/or pore blockers to change the amount or rate of cell volume change on a change in environmental parameters such as osmolality, pH or temperature. Furthermore the effects of two or more agents which affect transport of components in or out of cells on one another may be determined by this technique. It is also possible to subject the cell suspension to a change in shear stress during the passage of the cell suspension through the sensing zone by changing the flow rate through the sensor, without changing any of the other environmental parameters or in conjunction with a change in other environmental parameters. A change in the shear stress may affect the shape of the cell and thus the electrical, optical or other property which is measured by the sensor. Monitoring such a change in the deformation of cells may be of value. In particular, it may be of value to monitor the change in deformability upon changes imposed by disease or, artificially by changing other environmental parameters, such as chemical components of the suspending medium, pH, temperature or osmolality.

Preferably, the data processing means comprises the internal microprocessor of a personal computer.

When full data are available on the distribution of cell size in a particular population of cells subjected to hemolytic shock in a wide range of hypotonic solutions, at osmolalities just below the critical osmolality causing lysis, a gap in the populations is visible. On a 3-D plot or an alternative way of representing the data such as a contour map, the ghost cells are clearly visible and the unruptured cells are clearly identifiable, but between them there is a region defined by, for example, osmolality and cell size where the cells are widely distributed. The existence of this phenomenon, which has been termed “ghost gap”, has not previously been recognized, and it has been discovered that the nature of this phenomenon varies with species and between healthy and diseased individuals of particular species. It is a measure of the degree of anisocytosis (size heterogeneity) and can be used in the measurement of the degree of poikilocytosis (shape heterogeneity) of the cell population, which is often used as the basis for classifying all anemia.

The measurements of the cell parameter changes may be stored and retrieved as voltage pulses and they may be displayed as individual dots on a display of voltage against the osmolality of the solution causing the parameter change. When observations are made using a suspension at a single tonicity, the resulting plot shows the frequency distribution of voltage by the intensity of the dots representing cells of the same volume.

The number of blood cells within each aliquot which are counted is typically at least 1000 and the cell-by-cell data is then used to produce an exact frequency distribution of size. Suitably this density can be made more visible by using different colours to give a three dimensional effect, similar to that seen in radar rainfall pictures used in weather forecasting. Alternatively, for a single solution of any tonicity, the measured parameter change could be displayed against the number of individual cells showing the same change. In this way a distribution of cell volume or voltage in a particular tonicity of given osmolality can be obtained.

The method of the WO 97/24601 disclosure may be further improved by, instead of subjecting portions of a sample each to one of a series of hypotonic solutions of different osmolalities to form the individual aliquots, the sample is fed continuously into a solution, the osmolality of which is changed continuously to produce a continuous gradient of aliquots for passage through the sensing zone. Preferably, identical portions of the sample under test are subjected to solutions of each osmolality throughout the range under test after the same time from imposition of the environmental parameter change to the time of passage through the sensing zone. This technique ensures that the cells are subjected to the exact concentration which cause critical changes in that particular sample. Further, an effect of feeding the sample under test into a continuously changing osmolality gradient, is to obtain measurements which are equivalent to treating one particular cell sample with that continuously changing gradient. This technique is the subject of WO 97/24529.

Further, in the WO 97/24601 disclosure, it is possible to examine a particular blood sample at various intervals of time and compare the sets of results to reveal dynamic changes in cell function.

These dynamic changes have revealed that cells slowly decrease their ability to function over time, but they also change in unexpected ways. The size and shape of the cells in a blood sample change in a complex, non-linear but repeatable way, repeating some of the characteristic patterns of change over the course of days and on successive testing. The patterns, emerging over time, show similarity among like samples and often show a characteristic wave motion. The pattern of change may vary between individuals reflecting the health of the individual, or the pattern may vary within a sample. Thus a sample that is homogeneous when first tested may split into two or several sub-populations which change with time and their existence can be detected by subjecting the sample to a wide range of different tonicities and recording the cell size in the way described.

If the entire series of steps are repeated at timed intervals on further aliquots of the original sample and the resulting property change is plotted against osmolality, time and frequency distribution, a four-dimensional display, is obtained which may be likened to a changing weather map. The rate of change of the property in relation to the time taken to perform each test must be such that any changes which occur during the test must not substantially affect the results.

APPENDIX C: TABLE 7

| | | N | DEATH | | P0 | P0 | P0′ | SURVIVAL | SD | VI |
|---|---|---|---|---|---|---|---|---|---|---|
| DIAGNOSIS | N | (died) | % | SD P0 | (TOTAL) | (died) | interdecile | MONTHS | SURVIVAL | VOL Inc |
| Abdominal aortic | 19 | 8 | 42.1 | 24.6 | 143.7 | 140.2 | 137.7 | 61.8 | 49.3 | 68.5 |
| aneurysm | | | | | | | | | | |
| AIDS | 4 | 2 | 50.0 | 13.6 | 135.4 | 124.1 | 128.1 | 2.5 | 3.5 | 64.3 |
| Alcohol xs | 30 | 10 | 33.3 | 15.2 | 135.5 | 127.2 | 126.1 | 43 | 42.8 | 102.9 |
| Alpha 1 antitrypsin | 3 | 0 | 0.0 | 25.2 | 121.1 | | | | | 81 |
| deficiency | | | | | | | | | | |
| Amyloid | 3 | 1 | 33.3 | 5.7 | 136.7 | 130.1 | | 6 | | 120 |
| Anemia all | 387 | 100 | 25.8 | 13.6 | 140.4 | 138.8 | 138.7 | 81.9 | 42 | 86 |
| Anemia aplastic | 9 | 4 | 44.4 | 9.6 | 140.5 | 139.3 | 137.4 | 20.3 | 24.5 | 78.2 |
| Anemia elliptocytosis | 5 | 0 | 0.0 | 13.3 | 130.5 | | | | | 95.3 |
| Anemia Fe diff | 103 | 30 | 29.1 | 10.3 | 140.6 | 137.8 | 134 | 86.9 | 84.4 | 95 |
| Anemia megaloblastic | 3 | 1 | 33.3 | 5.8 | 129.7 | 133 | | 147 | — | 136 |
| Anemia | 2 | 1 | 50.0 | 2.1 | 148.5 | 148.5 | | 6.5 | 4.9 | |
| microangiopathetic | | | | | | | | | | |
| Anemia sideroblastic | 8 | 4 | 50.0 | 11.8 | 137.7 | 135 | 136.7 | 15.5 | 16.7 | 96.2 |
| Anemia, hemolytic | 30 | 6 | 20.0 | 12.5 | 147.2 | 149.4 | 152.7 | 117.2 | 154.8 | 60 |
| Anemia, hemolytic: | 5 | 0 | 0.0 | 10.9 | 135.6 | | | — | — | 122 |
| g6pd | | | | | | | | | | |
| Anemia, hemolytic: | 4 | 1 | 25.0 | 4.3 | 155.8 | 155 | | 2 | — | 45.1 |
| AIHA | | | | | | | | | | |
| Angina stable | 26 | 6 | 23.1 | 8.4 | 142.8 | 142.1 | 141.5 | 137.7 | 73 | 69 |
| Angina unstable | 38 | 17 | | 8.6 | 148.5 | 144.8 | 144.6 | 82.4 | 52.6 | 63.5 |
| Aortic stenosis | 9 | 3 | 33.3 | 9.9 | 139.8 | 139 | 140.6 | 19 | 30.3 | 72.5 |
| Aortic valve disease all | 19 | 6 | | 9.1 | 144.1 | 141.4 | 139.8 | 37.2 | 42.5 | 76.4 |
| other | | | | | | | | | | |
| Aortic valve repair | 12 | 5 | 41.7 | 9.7 | 141.8 | 140.9 | 136.8 | 43.6 | 44.2 | 84.3 |
| Aorto femoral bypass | 4 | 1 | | 15.1 | 134.7 | 125 | | 13 | — | 123 |
| Appendicitis acute | 8 | 2 | 25.0 | 6.7 | 143.8 | 135.4 | 128 | 85.5 | 118.1 | 76.9 |
| Arteriovenous | 2 | 0 | | 3.2 | 146.7 | | | | | 67 |
| malformation | | | | | | | | | | |
| Arthritis | 14 | 2 | | 9.8 | 144.4 | 139.5 | 147 | 183 | 60.8 | 100 |
| Artrioseptal defect | 3 | 0 | 0.0 | 0.8 | 136.1 | | | | | 68 |
| Artrioseptal defect post | 1 | 0 | | | 155 | | | | | 94 |
| repair | | | | | | | | | | |
| Asbestosis | 2 | 0 | 0.0 | 0 | 142 | | | | | 67 |
| ASD | 8 | 0 | | 6.6 | 149.3 | | | | | 61.7 |
| Asthma | 62 | 4 | 6.5 | 7.2 | 141.4 | 141.8 | 140.7 | 54.8 | 55 | 72.9 |
| Ataxia | 8 | 4 | 50.0 | 9.4 | 147.9 | 145.4 | 141.6 | 95 | 39.1 | 63.3 |
| Atrial fibrillation | 18 | 9 | 50.0 | 8.7 | 146.9 | 152.3 | 153 | 68.7 | 46.9 | 68 |
| Atrial flutter | 5 | 1 | 20.0 | 12.1 | 142.6 | 131.6 | | 122 | — | 66 |
| AUO | 44 | 13 | 29.5 | 10.2 | 138.6 | 134.8 | 135.7 | 97 | 96 | 62.6 |
| Avascular necrosis | 3 | 0 | 0.0 | 2.9 | 150.7 | — | | — | — | |
| Benign prostatic | 4 | 2 | 50.0 | 6.3 | 144.7 | 140.1 | 137 | 47 | 29.7 | 61.9 |
| hypertrophy | | | | | | | | | | |
| Bile duct obstruction | 4 | 2 | 50.0 | 14.1 | 136.6 | 125.6 | 130.1 | 58 | 24 | |
| Bladder stone | 2 | 1 | 50.0 | 1 | 139 | 138.3 | | 102 | — | 73 |
| Bladder tumor | 6 | 4 | 66.7 | 9.6 | 149.9 | 152.3 | 151.9 | 56.3 | 48.6 | 62.1 |
| Bleeding pr | 10 | 5 | 50.0 | 17 | 134.6 | 121.6 | 123.3 | 120.4 | 113.8 | 70.4 |
| Blood donors | 902 | 1 | 0.1 | 6.6 | 146.5 | 140 | | 187 | — | 81.5 |
| Brain tumor | 47 | 7 | 14.9 | 9.5 | 146.8 | 144.5 | 145.3 | 29 | 26.5 | 62.1 |
| Bronchiectasis | 5 | 1 | 20.0 | 6.4 | 142 | 133 | | 36 | | 60.8 |
| Bronchitis | 10 | 2 | 20.0 | 6.3 | 144.7 | 141.5 | 135 | 32 | 24 | 69.9 |
| Burns major | 1 | 0 | 0.0 | | 162 | | | | | 56 |
| ca basal cell carcinoma | 3 | 0 | 0.0 | 2 | 160.9 | | | | | 44.8 |
| ca bladder | 24 | 13 | 54.2 | 13 | 142.7 | 141.7 | 141.8 | 76.8 | 119.5 | 79.7 |
| ca breast | 43 | 14 | 32.6 | 11 | 144.2 | 139.4 | 139.4 | 94 | 101.7 | 95.3 |
| ca bronchus | 49 | 20 | 40.8 | 11.2 | 140.4 | 137.9 | 138.6 | 50 | 22 | 108.9 |
| ca carcinoid | 8 | 4 | 50.0 | 9.1 | 132.4 | 138.5 | 138 | 82.3 | 156.5 | 121 |
| ca colon | 162 | 31 | 19.1 | 15.2 | 137.1 | 132.2 | 132 | 48.7 | 77.8 | 105 |
| ca common bile duct | 6 | 4 | 66.7 | 15.9 | 121.5 | 115.8 | 122.7 | 4.5 | 3.3 | 121 |
| ca kidney | 6 | 4 | 66.7 | 20.2 | 136.9 | 129.6 | 128.8 | 7 | 7.7 | 105.1 |
| ca lung | 51 | 17 | 33.3 | 8.6 | 142.6 | 147.5 | 148.2 | 42.5 | 60 | 71.2 |
| ca malignant melanoma | 13 | 5 | 38.5 | 10.7 | 149.8 | 139.4 | 137.3 | 16.6 | 28.5 | 72.7 |
| ca nos | 13 | 4 | 30.8 | 8.5 | 139.8 | 140.2 | 139.3 | 12.3 | 6.9 | |
| ca oes | 16 | 7 | 43.8 | 16.9 | 140.8 | 132.6 | 133.1 | 37.8 | 70.1 | 87.6 |
| ca ovary | 25 | 13 | 52.0 | 17.9 | 137.5 | 133.4 | 132.3 | 29.6 | 76.8 | 90.9 |
| ca pancreas | 10 | 7 | 70.0 | 20.4 | 131.4 | 130.5 | 126.5 | 9.7 | 12.3 | 110.5 |
| ca prostate | 32 | 12 | 37.5 | 12 | 144.8 | 140.5 | 141.5 | 45.3 | 50.4 | 80.5 |
| ca rectum | 17 | 4 | 23.5 | 10.8 | 153.1 | 153.7 | 148.6 | 66.7 | 76.2 | 93 |
| ca stomach | 47 | 19 | 40.4 | 11.5 | 143.9 | 132.7 | 132.6 | 14.9 | 21.6 | 110.9 |
| ca testis | 3 | 0 | 0.0 | 5.9 | 142.3 | | | | | 102 |
| ca thyroid | 3 | 1 | 33.3 | 16.1 | 159 | 144 | | 9 | | 96 |
| ca ukp | 7 | 6 | 85.7 | 20.1 | 132.5 | 131.3 | 127.6 | 14 | 18.9 | 87.1 |
| ca uterus | 6 | 3 | 50.0 | 14.9 | 145.5 | 153.9 | 148.9 | 11 | 7.5 | 97.4 |
| ca? | 25 | 13 | 52.0 | 11 | 137.3 | 134.9 | 133.5 | 44 | 71.4 | 92.6 |
| Carcinomatosis | 6 | 1 | 16.7 | 10 | 148.2 | 150 | | 1 | | 124 |
| Cardiac arrest | 8 | 2 | 25.0 | 17.1 | 132.9 | 110.2 | 94 | 10 | 9.9 | 99.6 |
| Cardiac dysthrythmia | 11 | 4 | 36.4 | 8.3 | 143.2 | 147 | 144.7 | 25 | 34.1 | 66.2 |
| Carotid stenosis | 3 | 2 | 66.7 | 5 | 154.8 | 152.3 | 155 | 55.5 | 60.1 | 83.6 |
| Celiac | 103 | 6 | 5.8 | 11.5 | 137.5 | 130 | 130.4 | 128.2 | 94.4 | 58 |
| Cerebro-vascular | 35 | 21 | 60.0 | 9.6 | 143 | 141.3 | 141 | 51.2 | 67.5 | 72.5 |
| accident | | | | | | | | | | |
| Cholecystectomy | 9 | 3 | 33.3 | 4.4 | 133.2 | 134.7 | 137.1 | 95.3 | 84.2 | 111.5 |
| Cholecystitis | 9 | 1 | 11.1 | 15.8 | 149.5 | 176 | A | 7 | | 77.4 |
| Chronic obstructive | 101 | 19 | 18.8 | 13 | 143.7 | 145.7 | 144.9 | 41.3 | 48.5 | 68.1 |
| airway disease | | | | | | | | | | |
| Cirrhosis all | 30 | 10 | 33.3 | 15.9 | 132.8 | 125.2 | 124 | 40.6 | 50.3 | 98.4 |
| Claudication | 5 | 2 | 40.0 | 7.2 | 154.7 | 155.1 | 165.3 | 177 | 87.7 | 83.9 |
| Coagulopathy | 3 | 2 | 66.7 | 5.9 | 149.7 | 146.5 | 144.4 | 85.5 | 37.5 | 64.9 |
| Congestive heart | 43 | 20 | 46.5 | 19.6 | 146.4 | 147.9 | 149.2 | 55.6 | 70.1 | 68.3 |
| failure | | | | | | | | | | |
| Control | 216 | 11 | 5.1 | 7.1 | 142.8 | 138.1 | 137.9 | 216.1 | 97.5 | 96.4 |
| Control women | 6 | 0 | 0.0 | 5.2 | 136 | | | | | |
| Cord | 17 | 0 | 0.0 | 8.4 | 125.7 | | | | | 125.4 |
| Coronary artery disease | 48 | 15 | 31.3 | 11.2 | 141.8 | 141.4 | 142 | 94.3 | 122.3 | 73.5 |
| Crohns | 23 | 3 | 13.0 | 11.7 | 145.3 | 128.7 | 129.5 | 166.3 | 98.7 | 96.5 |
| Cushings + thal maj | 3 | 0 | 0.0 | 4.2 | 148.8 | | | | | 59.4 |
| Cystic fibrosis | 103 | 5 | 4.9 | 9.1 | 137.7 | 138.9 | 139.2 | 103 | 119.2 | 87.1 |
| Cystic fibrosis hz | 10 | 2 | 20.0 | 5.4 | 142.6 | 140.5 | 138.1 | 173 | 113.1 | |
| Cystic fibrosis mec | 1 | 1 | 100.0 | | 95 | 95 | | 311 | | 118 |
| ileus | | | | | | | | | | |
| D & C | 9 | 2 | 22.2 | 7.9 | 135.6 | 140 | 140 | 246.5 | 123.7 | 81 |
| Deep vein thrombosis | 17 | 3 | 17.6 | 16.4 | 138.3 | 141 | 135.5 | 60.3 | 91.8 | 101.7 |
| Dehydration | 12 | 5 | 41.7 | 11.8 | 140.9 | 140.2 | 138.3 | 7.8 | 6.2 | 65.4 |
| Dementia | 5 | 4 | 80.0 | 9 | 145.2 | 143.1 | 144.1 | 23 | 16.3 | 72.2 |
| Diabetes mellitus | 14 | 6 | 42.9 | 10.9 | 145.5 | 142.9 | 143.1 | 104 | 86.1 | 92.8 |
| Diaphragmatic hernia | 5 | 0 | 0.0 | 9.4 | 129.2 | | | | | 121.8 |
| Disc lesion | 15 | 2 | 13.3 | 6.9 | 149.4 | 147.7 | 150.4 | 3 | 4.2 | 58.4 |
| Down's syndrome | 3 | 0 | 0.0 | 11.1 | 148.8 | | | | | 89.8 |
| Duodenal ulcer | 10 | 3 | 30.0 | 9.8 | 139.5 | 139.7 | 145 | 123.3 | 137.3 | 87.2 |
| Dysphagia | 3 | 1 | 33.3 | 7.3 | 128.8 | 135.5 | | 13 | | 86.5 |
| Dyspnoea | 115 | 14 | 12.2 | 9.1 | 141.8 | 140.8 | 140.7 | 53.8 | 79.3 | 70.8 |
| Emphesema | 11 | 2 | 18.2 | 4.8 | 141.4 | 145.5 | 148 | 74 | 65.1 | 67.9 |
| Epilepsy | 25 | 4 | 16.0 | 11.8 | 144.2 | 142.5 | 144.7 | 102.5 | 92.2 | 70 |
| Esophagitis | 3 | 1 | 33.3 | 11 | 137.9 | 150.5 | | 86 | | |
| Femoral popliteal | 11 | 6 | 54.5 | 11.9 | 141.1 | 141.4 | 142.2 | 70 | 50.8 | 85 |
| bypass | | | | | | | | | | |
| Fibroadenoma breast | 3 | 0 | 0.0 | 7.5 | 154.2 | | | | | 89.6 |
| Fractures | 64 | 15 | 23.4 | 10.5 | 141.1 | 143.4 | 143.4 | 63.7 | 68.3 | 80.4 |
| Gall stones | 14 | 3 | 21.4 | 5.8 | 150.8 | 153.7 | 154.6 | 40.7 | 33 | 63 |
| Gangrene | 4 | 1 | 25.0 | 20.7 | 140.4 | 161.7 | | 39 | | 70.5 |
| Gastric ulcer | 6 | 2 | 33.3 | 5.6 | 156.7 | 158.5 | 160 | 8 | 5.7 | 82 |
| Gastro-intestinal bleed | 55 | 18 | 32.7 | 14.5 | 140.3 | 134.7 | 135.8 | 85.7 | 71.3 | 85.9 |
| Glandular fever | 5 | 2 | 40.0 | 5.4 | 151.4 | 153.5 | 157 | 8.5 | 12 | 6 |
| Guillain Barre | 3 | 0 | 0.0 | 6.7 | 136.7 | | | | | 77 |
| Hb AC | 2 | 0 | 0.0 | 1.8 | 133.25 | | | | | 87.5 |
| Hb AE etc | 8 | 0 | 0.0 | 16.8 | 114.4 | | | | | 78.1 |
| Hb Agononi | 1 | 0 | 0.0 | | 109 | | | | | 84 |
| hb CC | 16 | 0 | 0.0 | 15.4 | 104.8 | — | | — | — | 75.6 |
| Hb H disease | 2 | 0 | 0.0 | 16.1 | 103.7 | | | | | 66 |
| hb S b thal | 4 | 1 | 25.0 | 7.5 | 101.3 | 97 | | 230 | — | 43 |
| Hb SC | 7 | 0 | 0.0 | 9.8 | 95.2 | | | | | 115 |
| hb SS all | 108 | 4 | 3.7 | 65.3 | 114.8 | 117.3 | 118 | 69.9 | | 60 |
| hb ss crisis | 20 | 2 | 10.0 | 10.6 | 115.7 | 127.6 | 140.1 | 121.5 | 24.7 | 42.7 |
| hb ss no crisis | 81 | 2 | 2.5 | 15.3 | 115.2 | 107 | 95 | 28.5 | 40.3 | 63.7 |
| Hb th maj or int never | 1 | 0 | 0.0 | | 99 | | | | | 66 |
| tx | | | | | | | | | | |
| Hb thal E various | 7 | 0 | 0.0 | 17.7 | 115.6 | | | | | 75.3 |
| hb thal I txed | 2 | 0 | 0.0 | 8.6 | 131.5 | — | | | | 38.6 |
| Hb thal int | 12 | 0 | 0.0 | 13.2 | 86.3 | | | | | 63.1 |
| hb thal maj ALL | 75 | 3 | 4.0 | 12.2 | 134.9 | 139.7 | 144.5 | 147 | 81.4 | 61.1 |
| hb thal maj never tx | 2 | 0 | 0.0 | 5.8 | 113.1 | | | | | 70 |
| hb thal maj pre tx | 4 | 0 | 0.0 | 14.6 | 136.5 | | | | | 61.5 |
| Hb thal major txed | 15 | 0 | 0.0 | 6.7 | 136.2 | | | | | 81.9 |
| Hematuria | 10 | 5 | 50.0 | 9.1 | 145.6 | 144.4 | 142.5 | 120 | 119.2 | 70.3 |
| Hemoglobin AS | 9 | 1 | 11.1 | 9.9 | 125.9 | 130.1 | | 355 | | 97 |
| Hemolytic uremic | 2 | 1 | 50.0 | 9.1 | 110.4 | 116.9 | | 4 | | 110.3 |
| syndrome | | | | | | | | | | |
| Hepatitis | 11 | 2 | 18.2 | | 131.9 | 120.1 | 100 | 94.5 | 26.2 | 78.8 |
| Hereditary | 17 | 3 | 17.6 | 11.9 | 152.6 | 140.1 | 136.6 | 81.7 | 117 | 64.8 |
| spherocytosiss | | | | | | | | | | |
| Hereditary | 3 | 1 | 33.3 | 28.4 | 139.7 | 45.3 | | 95 | — | 45 |
| telangiectasia | | | | | | | | | | |
| Hernia | 17 | 8 | 47.1 | 16.4 | 146.9 | 142.2 | 142.5 | 129 | 89.7 | 70.7 |
| Herpes simplex | 1 | 0 | 0.0 | | 148 | | | | | 95 |
| Herpes zoster | 5 | 1 | 20.0 | 6.7 | 148.5 | 140 | | 49 | | 87.6 |
| Herpes zoster | 4 | 0 | 0.0 | 4.8 | 150.6 | | | | | 86.3 |
| Hgb alpha 1 thal | 3 | 0 | 0.0 | 20.6 | 123.3 | | | | | 103 |
| Hgb thal E hz | 4 | 0 | 0.0 | 7.5 | 123.8 | | | | | 77.5 |
| Hip replacement | 7 | 5 | 71.4 | 10.4 | 140.9 | 139.4 | 137.8 | 183 | 63.3 | 100 |
| Hiv | 8 | 5 | 62.5 | 7.5 | 139.1 | 136.5 | 134.4 | 51.2 | 47.1 | 67.6 |
| Hurlers | 1 | 0 | 0.0 | | 130.1 | | | | | |
| Hyaline membrane | 4 | 0 | 0.0 | 6.3 | 118.1 | | | | | 112.3 |
| disease | | | | | | | | | | |
| Hydrocephalus | 10 | 3 | 30.0 | 12.7 | 139.9 | 149.9 | 150.2 | 18.3 | 15 | 60 |
| Hypersplenism | 7 | 1 | 14.3 | 9.8 | 132.4 | 131.9 | | 39 | | 71 |
| Hypertension | 21 | 3 | 14.3 | 8.1 | 144.5 | 135.7 | 130.6 | 167.3 | 144.2 | 87.8 |
| Hypertension | 25 | 0 | 0.0 | 3.8 | 146.1 | — | | — | — | |
| malignant | | | | | | | | | | |
| Hypotension | 2 | 0 | 0.0 | 11.2 | 161.1 | | | | | 78.7 |
| Hysterectomy | 14 | 4 | 28.6 | 9.6 | 140 | 137.3 | 139.3 | 124.5 | 92.5 | 75 |
| Idiopathic | 39 | 4 | 10.3 | 9.8 | 144.1 | 150 | 149.3 | 84.8 | 23 | 56 |
| thrombocytopenic | | | | | | | | | | |
| purpura | | | | | | | | | | |
| Infected | 5 | 1 | 20.0 | 15.1 | 144.4 | 141.2 | | | | 62.9 |
| Interstitial lung disease | 3 | 0 | 0.0 | 6.7 | 141 | | | | | 76.8 |
| Intest obstructn | 12 | 3 | 25.0 | 15.6 | 138.3 | 135.3 | 140 | 107.3 | 110.6 | 94.1 |
| Ischemic bowel disease | 4 | 1 | 25.0 | 16.5 | 135.8 | 123 | | 2 | — | 112 |
| Jaundice | 9 | 2 | 22.2 | 9.2 | 132.7 | 134 | 142 | 2.5 | 3.5 | 93.6 |
| Laminectomy | 6 | 0 | 0.0 | 11.6 | 143.7 | | | | | |
| Leukemia acute | 5 | 2 | 40.0 | 6.8 | 135.8 | 140.3 | 142 | 0 | 0 | 75.9 |
| Leukemia acute | 25 | 15 | 60.0 | 16.5 | 143 | 138.9 | 139.1 | 10.7 | 12.7 | 98 |
| myeloid | | | | | | | | | | |
| Leukemia ALL | 2 | 0 | 0.0 | 2.1 | 153.5 | | | | | 90 |
| Leukemia AML | 25 | 15 | 60.0 | 16.5 | 140.6 | 138.9 | 139.1 | 10.7 | 12.7 | 98.5 |
| Leukemia CGL | 5 | 2 | 40.0 | 6.7 | 143.9 | 143 | 152 | 4.5 | 4.9 | 83.7 |
| Leukemia CLL | 13 | 5 | 38.5 | 12.4 | 146.3 | 147.9 | 148 | 18.4 | 28.5 | 100.5 |
| Leukemia CML | 2 | 1 | 50.0 | 2.8 | 135 | 133 | | 81 | | 105 |
| Leukemia nos | 11 | 3 | 27.3 | 13.6 | 137.8 | 130.1 | 132.9 | 27.3 | 9.3 | 77 |
| Leukemia total | 71 | 34 | 47.9 | 17.3 | 141.8 | 139.7 | 139 | 17 | 26.8 | 88.3 |
| Liver failure | 25 | 10 | 40.0 | 11.2 | 130.1 | 130.4 | 128.9 | 3.7 | 35 | 78.4 |
| Lobectomy | 2 | 0 | 0.0 | 0.7 | 137.5 | — | | — | — | 62 |
| Lung lesion/nodule | 29 | 5 | 17.2 | 11.6 | 142.8 | 148.8 | 150.1 | 25.6 | 28.2 | 70 |
| Lung tx | 4 | 1 | 25.0 | 10.4 | 133.8 | 131.4 | | 22 | — | 76.6 |
| Lver transplant | 9 | 1 | 11.1 | 11.3 | 130.3 | 112.6 | | 142 | | 79 |
| Lymphoma H | 20 | 6 | 30.0 | 12.3 | 141.1 | 134.5 | 132.2 | 32 | 42.8 | 83.6 |
| Lymphoma NH | 36 | 12 | 33.3 | 11.1 | 144.5 | 139.8 | 139.2 | 20.3 | 32 | 76.4 |
| Malaise | 17 | 2 | 11.8 | 5.9 | 143.1 | 148.5 | 154 | 189 | 116 | 91 |
| Malaria | 11 | 0 | 0.0 | 15.8 | 143.8 | — | | — | — | 64.7 |
| Meconium ileus (cystic | 1 | 1 | 100.0 | — | 95 | 95 | | 311 | — | 118 |
| fibrosis) | | | | | | | | | | |
| Menorrhagia | 47 | 1 | 2.1 | 13.4 | 140.7 | 144 | | 65 | 0 | 84.5 |
| Mitral valve disease | 17 | 2 | 11.8 | 7.1 | 141.9 | 148.5 | 157 | 152.5 | 159.1 | 85.2 |
| Mitral valve disease | 33 | 4 | 12.1 | 11.2 | 143.6 | 145.7 | 147.7 | 112 | 118.1 | 78 |
| Motor neurone disease | 3 | 3 | 100.0 | 6.5 | 152.4 | 152.4 | 155.5 | 42.7 | 39.5 | 64.6 |
| mult myeloma | 4 | 0 | 0.0 | 8.1 | 141.6 | — | | — | — | 102 |
| mult myeloma | 14 | 17 | 121.4 | 10.2 | 143.2 | 137.3 | 134.5 | 27.9 | 18.5 | 80.8 |
| Multiple sclerosis | 8 | 2 | 25.0 | 9.7 | 145.5 | 152.9 | 147 | 6 | 8.5 | 80 |
| Muscular dystrophy all | 112 | 11 | 9.8 | 8.9 | 143 | 142.4 | 141.4 | 104.3 | 33.9 | 71 |
| Muscular dystrophy | 7 | 1 | 14.3 | 19.8 | 139 | 138.2 | | 56 | | 55.9 |
| beckers | | | | | | | | | | |
| Muscular dystrophy | 4 | 2 | 50.0 | 9.1 | 134.5 | 141 | 137 | 85.5 | 17.7 | 91.2 |
| duchenne | | | | | | | | | | |
| Muscular dystrophy | 65 | 4 | 6.2 | 7.1 | 144.6 | 140.6 | 136.7 | 118.8 | 43.4 | 69 |
| nos | | | | | | | | | | |
| Muscular dystrophy | 4 | 2 | 50.0 | 7.1 | 146.1 | 150.6 | 151 | 123.5 | 10.6 | 69.6 |
| sma | | | | | | | | | | |
| Muscular dystrophy; | 20 | 1 | 5.0 | 5.4 | 137.7 | 139.3 | | 89 | | 69.3 |
| myotonic | | | | | | | | | | |
| Myasthenia gravis | 4 | 1 | 25.0 | 4 | 142.2 | 136.6 | | 136 | — | 68 |
| Myelodysplasia | 115 | 5 | 4.3 | 19 | 134.1 | 140.5 | 140.4 | 19.2 | 20 | 72 |
| Myelofibrosis | 24 | 11 | 45.8 | 15 | 137.5 | 135.6 | 135.2 | 11.9 | 14.7 | 77.8 |
| Myocardial infarct | 18 | 7 | 38.9 | 9.8 | 144.1 | 138.7 | 136 | 123.5 | 94.8 | 105 |
| Neonatal | 12 | 1 | 8.3 | 13.4 | 116.3 | 106 | | 0 | — | 106 |
| Neoplasm benign | 13 | 0 | 0.0 | 8.7 | 141.2 | — | | — | | 70.2 |
| Neoplasm glioblastoma | 6 | 1 | 16.7 | 11.8 | 137.4 | 156.7 | | 16 | | 73.6 |
| New born | 110 | 1 | 0.9 | 13.9 | 129.3 | 135.2 | | 17 | — | 87 |
| Osteoarthritis | 28 | 6 | 21.4 | 8.7 | 148 | 143 | 139.8 | 84.3 | 44.4 | 68.5 |
| ovarian cyst | 6 | 1 | 16.7 | 7.1 | 146.4 | 136 | | 15 | — | 77 |
| Pancreatitis | 13 | 1 | 7.7 | 11.8 | 135.9 | 136 | | 87 | — | 87.6 |
| Pancytopenia | 3 | 1 | 33.3 | 10.4 | 140.4 | 151.3 | | 5 | | 68.2 |
| Parkinsons | 2 | 0 | 0.0 | 4.9 | 143.5 | — | | | | 72 |
| Peripheral vascular | 21 | 12 | 57.1 | 13.5 | 146.2 | 143.4 | 142.2 | 40.1 | 33.9 | 64 |
| disease | | | | | | | | | | |
| Pernicious anemia | 6 | 0 | 0.0 | 8.6 | 143.6 | — | | | | 81 |
| Platelets giant | 3 | 1 | 33.3 | 8 | 110.8 | 120 | | 5 | | 76 |
| Platelets small | 9 | 1 | 11.1 | 7 | 145.1 | 136 | | 13 | | 67 |
| PN- | 41 | 21 | 51.2 | 11.6 | 144.4 | 147.7 | 148.7 | 40.2 | 47.1 | 65.2 |
| Polycythemia vera | 50 | 17 | 34.0 | 7.2 | 145.7 | 149.8 | 149.3 | 71.8 | 47.2 | 104.7 |
| Polymyalgia | 3 | 1 | 33.3 | 1.2 | 139.4 | 138 | | 129 | | |
| Polyneuropathy | 2 | 2 | 100.0 | 2.8 | 152 | 152 | 154 | | | 58 |
| Pregnancy 0 | 68 | 2 | 2.9 | 10 | 148.9 | 151 | 147 | 291.5 | 9.2 | 55 |
| Pregnancy 1 | 19 | 0 | 0.0 | 7.3 | 150.2 | | | | | 55 |
| Pregnancy 2 | 14 | 2 | 14.3 | 9.5 | 142.8 | 148 | 156 | 121 | 155 | 53 |
| Pregnancy 3 | 65 | 3 | 4.6 | 11 | 146.7 | 130.5 | 132 | 88.7 | 56 | 50.4 |
| Pregnancy 4 | 13 | 0 | 0.0 | 6 | 145.8 | — | | — | — | 62.3 |
| Pregnancy 5-7 | 14 | 0 | 0.0 | 8.2 | 146.8 | — | | — | — | 47.4 |
| Pregnancy 8, 9 | 4 | 0 | 0.0 | 4.3 | 148.1 | | | | | 44.4 |
| Pregnancy an nos | 53 | 1 | 1.9 | 13.8 | 142.3 | 131 | | 85 | | 70.3 |
| Pregnancy L | 50 | 0 | 0.0 | 8 | 149.4 | | | | | 55 |
| Pregnancy pn nos | 47 | 2 | 4.3 | 7.9 | 148.2 | 140.5 | 149 | 159 | 67.2 | |
| Pregnancyan 10-20/40 | 10 | 1 | 10.0 | 12.5 | 147.2 | 145 | | 288 | — | 47 |
| Pregnancyan 20-29/40 | 15 | 0 | 0.0 | 15.6 | 149.7 | — | | — | — | 51.4 |
| Pregnancyan 21/40 | 5 | 0 | 0.0 | 8.5 | 148 | | | | | 48.5 |
| Pregnancyan 30-34/40 | 18 | 0 | 0.0 | 10.1 | 145.8 | | | | | 62.4 |
| Pregnancyan 32/40 | 18 | 0 | | 9.8 | 146 | | | | | 52.7 |
| Pregnancyan 35-36/40 | 24 | 2 | 8.3 | 8.3 | 147.8 | 140 | 143 | 67 | 52.3 | 50 |
| Pregnancyan 37-39/40 | 33 | 0 | 0.0 | 9.6 | 148 | | | | | 46.4 |
| Pregnancyan 40+ | 7 | 0 | 0.0 | 15.5 | 144 | | | | | 44.2 |
| Pregnancyan 40 n bp | 23 | 0 | 0.0 | 10.6 | 150.4 | | | | | 54.4 |
| Pregnancyan 40-42/40 + | 30 | | 0.0 | 8.8 | 147.5 | | | | | 53.6 |
| BP | | | | | | | | | | |
| Pulmonary embolus not | 12 | 1 | 8.3 | 11.9 | 139.9 | 140 | | 153 | 7.1 | 78 |
| on warf | | | | | | | | | | |
| Pulmonary embolus on | 4 | 1 | 25.0 | 8.9 | 149.4 | 157 | | | | |
| warfarin | | | | | | | | | | |
| Pulmonary fibrosis | 4 | 0 | 0.0 | 8.1 | 143.4 | | | | | 71.7 |
| Pulmonary hypetension | 5 | 0 | 0.0 | 12.6 | 143.3 | | | | | 69.6 |
| Pyloric stenosis | 5 | 1 | 20.0 | 20.7 | 131.5 | 150 | | 10 | — | 118.4 |
| Pyrexia of unknown | 16 | 6 | 37.5 | 10.2 | 140.9 | 142.9 | 139.1 | 77.7 | 167 | 61 |
| origin | | | | | | | | | | |
| Quadriplegia | 5 | 0 | 0.0 | 10.1 | 139.8 | — | | — | — | 66.3 |
| Reiters | 2 | 1 | 50.0 | 3.6 | 137.5 | 140.1 | | 121 | | 79 |
| Renal failure chronic | 275 | 135 | 49.1 | 12.9 | 138.3 | 134.9 | 134.9 | 55.9 | 99.3 | |
| Renal failure: acute | 9 | 2 | | 11.9 | 140.2 | 136.5 | 133 | 24 | 24 | 81.5 |
| Renal stone | 13 | 3 | 23.1 | 7.8 | 152.1 | 146.7 | 141.4 | 81.7 | 68.5 | 66.1 |
| Renal transplant | 19 | 9 | 47.4 | 13.6 | 144.2 | 143.7 | 146.8 | 128 | 90.8 | 71.8 |
| Respiratory distress | 4 | 1 | 25.0 | 9.4 | 124.6 | 126 | | 0 | — | 104.5 |
| syndrome | | | | | | | | | | |
| Respiratory failure | 5 | 4 | 80.0 | 19.2 | 135 | 131.8 | 131.9 | 35 | 45.8 | 79.7 |
| Rheumatoid arthritis | 18 | 1 | 5.6 | 7.4 | 143.9 | 135 | | 157 | — | 106.8 |
| pen | | | | | | | | | | |
| Rheumatoid arthritis all | 63 | 5 | 7.9 | 9.3 | 142.2 | 134.5 | 133.2 | 82.8 | 50.2 | 100 |
| Rheumatoid arthritis au | 9 | 2 | 22.2 | 13.7 | 141.9 | 122 | 112 | 62 | 5.7 | 88.5 |
| Rheumatoid arthritis az | 3 | 0 | 0.0 | 9.2 | 145 | | | | | 115 |
| Rheumatoid arthritis st | 3 | 0 | 0.0 | 7.2 | 150.3 | — | | — | — | 111 |
| RT | 10 | 4 | 40.0 | 6.6 | 149.6 | 145.2 | 145.9 | 42 | 48.6 | 56.3 |
| Sarcoid | 17 | 0 | 0.0 | 11.2 | 137.7 | — | | — | — | 74.3 |
| Sarcoma | 5 | 3 | 60.0 | 13.6 | 135.8 | 133.7 | 131.4 | 62.7 | 104.2 | 71.1 |
| Satelitism | 1 | 0 | 0.0 | | 145.1 | | | | | 73 |
| Scleroderma | 6 | 0 | 0.0 | 5.5 | 144.9 | — | | — | — | 84 |
| Scoliosis | 4 | 0 | 0.0 | 5.1 | 147.3 | — | | — | — | 65.7 |
| Sepsis | 8 | 6 | 75.0 | 19 | 142 | 135.6 | 129.5 | 8.4 | 13.1 | 57 |
| Sleep apnoea | 5 | 0 | 0.0 | 8.5 | 145.4 | | | | | 74.4 |
| Spina bifida | 5 | 0 | 0.0 | 10.8 | 153.8 | | | | | 63.3 |
| Splenectomy | 8 | 1 | 12.5 | 9.2 | 148.2 | 140.1 | | 56 | | 92.7 |
| Sprue | 1 | 1 | 100.0 | | 115 | 115 | | 95 | | 58 |
| Stem cells | 4 | 0 | 0.0 | 10.4 | 120 | | | | | 55.8 |
| Subacute bacterial | 9 | 2 | 22.2 | 12.2 | 137.2 | 126 | 105 | 217.5 | 123.7 | 114.3 |
| endocarditis | | | | | | | | | | |
| Syncope | 5 | 0 | 0.0 | 12.4 | 137.7 | | | | | 79.8 |
| Systemic lupus | 7 | 0 | 0.0 | 5.5 | 147.2 | — | | — | — | 86.6 |
| erythethematosis | | | | | | | | | | |
| T's & As | 6 | 0 | 0.0 | 3.5 | 138.4 | | | | | 68 |
| Temperal arteritis | 4 | 3 | 75.0 | 6.2 | 147.1 | 145.2 | 142 | 242 | 34 | 100 |
| Thalassemia beta trait | 81 | 1 | 1.2 | 10.6 | 117.1 | 123.8 | | 201 | | 82.8 |
| Thrombocytopenia | 7 | 1 | 14.3 | 17.2 | 140.2 | 149 | | 174 | | 71 |
| Thrombocytosis | 5 | 0 | 0.0 | 12.3 | 133.9 | | | | | 63.5 |
| Thrombotic | 2 | 0 | 0.0 | 0.6 | 106 | | | | | 103.5 |
| thrombocytopenic | | | | | | | | | | |
| purpura | | | | | | | | | | |
| Thyrotoxicosis | 7 | 0 | 0.0 | 12.7 | 140.7 | | | | | 81.2 |
| Transient ischemic | 7 | 3 | 42.9 | 5 | 145.1 | 147.3 | 145.9 | 49.7 | 35.5 | 103.4 |
| attacks | | | | | | | | | | |
| Turp | 17 | 14 | 82.4 | 8.3 | 139.1 | 137.5 | 138.6 | 106.9 | 75.8 | 85.1 |
| Ulcerative colitis | 35 | 4 | 11.4 | 12.4 | 142 | 146.8 | 147 | 74.3 | 123 | 102.4 |
| Urinary retention | 8 | 5 | 62.5 | 11.4 | 150.2 | 149.4 | 153 | 69.4 | 54.3 | 58.2 |
| Uterine fibroids | 22 | 1 | 4.5 | 8.4 | 144.2 | 150 | | 158 | | 63 |
| Ventric tachy | 12 | 3 | 25.0 | 9.8 | 135.6 | 137.2 | 137 | 34.3 | 35.1 | 74 |
| Volvulus sigmoid | 3 | 1 | 33.3 | 7.1 | 133.3 | 132 | | 81 | | 111 |
| Waldenstrom's | 1 | 0 | 0.0 | | 122 | | | | | 80 |
| macroglobulinemia | | | | | | | | | | |

| | | N | DEATH | SHAPE | SHAPE | | | | | | |
|---|---|---|---|---|---|---|---|---|---|---|---|
| DIAGNOSIS | N | (died) | % | (SAVR) | (SI) | SAML | PYmax | Pymin | PLTS | MPV | MCV |
| Abdominal aortic aneurysm | 19 | 8 | 42.1 | | 15.3 | 578 | | | | | |
| AIDS | 4 | 2 | 50.0 | | 16.8 | | | | | | |
| Alcohol xs | 30 | 10 | 33.3 | 1.62 | 16.7 | 635 | | | 233 | | 97.5 |
| Alpha 1 antitrypsin deficiency | 3 | 0 | 0.0 | 1.8 | | 530 | | | | | |
| Amyloid | 3 | 1 | 33.3 | | | 627 | | | | | 82.7 |
| Anemia all | 387 | 100 | 25.8 | 1.7 | 15.4 | 461 | | | | | |
| Anemia aplastic | 9 | 4 | 44.4 | 1.7 | 16.5 | 435 | | | 28 | | 89.4 |
| Anemia elliptocytosis | 5 | 0 | 0.0 | 1.8 | | 382 | | | 275 | | 78.8 |
| Anemia Fe diff | 103 | 30 | 29.1 | 1.7 | 16.4 | 439.6 | | | 355.6 | | 76.5 |
| Anemia megaloblastic | 3 | 1 | 33.3 | | | — | | | | | 79 |
| Anemia microangiopathetic | 2 | 1 | 50.0 | | | | | | | | |
| Anemia sideroblastic | 8 | 4 | 50.0 | 1.6 | | 425.8 | | | 205.8 | | 94.6 |
| Anemia, hemolytic | 30 | 6 | 20.0 | 1.6 | 14.8 | 498 | | | 238 | | 91 |
| Anemia, hemolytic: g6pd | 5 | 0 | 0.0 | | | | | | 350 | | 97 |
| Anemia, hemolytic: AIHA | 4 | 1 | 25.0 | 1.7 | 14.5 | 491.4 | | | 282 | | 94.7 |
| Angina stable | 26 | 6 | 23.1 | | 15.5 | 646 | | | 231.3 | | 86.6 |
| Angina unstable | 38 | 17 | | 1.6 | 16.6 | 581.2 | | | 181.4 | | 88.6 |
| Aortic stenosis | 9 | 3 | 33.3 | | 17.1 | 455 | | | 63.5 | | 112.9 |
| Aortic valve disease all other | 19 | 6 | | | 15.6 | 706 | | | 205 | | 89.2 |
| Aortic valve repair | 12 | 5 | 41.7 | | 15.4 | 706 | | | 205 | | 89.2 |
| Aorto femoral bypass | 4 | 1 | | — | — | — | | | 436 | | 94 |
| Appendicitis acute | 8 | 2 | 25.0 | | 16.9 | 704.1 | | | 237 | | 86.2 |
| Arteriovenous malformation | 2 | 0 | | | 16.7 | | 11 | 13.5 | | | |
| Arthritis | 14 | 2 | | — | — | — | | | — | | 86.3 |
| Artrioseptal defect | 3 | 0 | 0.0 | | 16.8 | | 11 | 16 | | | |
| Artrioseptal defect post repair | 1 | 0 | | | | | | | | | |
| Asbestosis | 2 | 0 | 0.0 | | 15.4 | | | | | | |
| ASD | 8 | 0 | | 1.59 | 14.7 | 414 | | | 150 | | 99.3 |
| Asthma | 62 | 4 | 6.5 | — | 15.2 | 606.3 | | | 254.4 | | 90.2 |
| Ataxia | 8 | 4 | 50.0 | — | 15.9 | — | | | 171 | | 90 |
| Atrial fibrillation | 18 | 9 | 50.0 | | 15.2 | 552 | | | 206 | | 91.4 |
| Atrial flutter | 5 | 1 | 20.0 | | 16.1 | 676 | | | 208 | | 86.6 |
| AUO | 44 | 13 | 29.5 | — | 16.3 | 714 | | | 246 | | 92.7 |
| Avascular necrosis | 3 | 0 | 0.0 | | | | | | | | |
| Benign prostatic hypertrophy | 4 | 2 | 50.0 | | 16.2 | 688 | | | 345 | | 96.1 |
| Bile duct obstruction | 4 | 2 | 50.0 | | | | | | | | 77 |
| Bladder stone | 2 | 1 | 50.0 | | 17.31 | 705 | | | 370 | | 90.6 |
| Bladder tumor | 6 | 4 | 66.7 | | 15.7 | 564.5 | | | 282 | | 84.7 |
| Bleeding pr | 10 | 5 | 50.0 | | 12.5 | 642 | | | 519.5 | | 87.3 |
| Blood donors | 902 | 1 | 0.1 | 1.5 | 15.5 | 463.5 | | | 290.4 | | 85.1 |
| Brain tumor | 47 | 7 | 14.9 | — | 14.7 | 574.8 | | | 294.8 | | 94.5 |
| Bronchiectasis | 5 | 1 | 20.0 | — | | | | | | | |
| Bronchitis | 10 | 2 | 20.0 | | 15.7 | | | | | | |
| Burns major | 1 | 0 | 0.0 | | 15.6 | 470 | | | | | |
| ca basal cell carcinoma | 3 | 0 | 0.0 | | 14.5 | | | | | | |
| ca bladder | 24 | 13 | 54.2 | | 15.6 | 470 | | | 201 | | 83 |
| ca breast | 43 | 14 | 32.6 | 1.68 | 15.6 | 616 | | | 333.3 | | 90 |
| ca bronchus | 49 | 20 | 40.8 | 1.7 | | 591.3 | | | 345 | | 86.9 |
| ca carcinoid | 8 | 4 | 50.0 | | | 659 | | | — | | 79 |
| ca colon | 162 | 31 | 19.1 | 1.6 | 15.6 | 635 | | | 260 | | 83.5 |
| ca common bile duct | 6 | 4 | 66.7 | | 16.9 | 713 | | | 33 | | 91.3 |
| ca kidney | 6 | 4 | 66.7 | | 14.23 | 607.4 | | | — | | 80 |
| ca lung | 51 | 17 | 33.3 | 1.7 | 15.5 | 545 | | | 396.6 | | 85 |
| ca malignant melanoma | 13 | 5 | 38.5 | 1.63 | 15.9 | 556.5 | | | 230 | | 90.3 |
| ca nos | 13 | 4 | 30.8 | | 15.4 | 596 | | | 250 | | 75.9 |
| ca oes | 16 | 7 | 43.8 | | 15 | 607 | | | — | | 90.6 |
| ca ovary | 25 | 13 | 52.0 | 1.6 | 16 | 554 | | | 396 | | 83.7 |
| ca pancreas | 10 | 7 | 70.0 | | 15.8 | 636 | | | 304 | | 82.5 |
| ca prostate | 32 | 12 | 37.5 | 1.61 | 15.8 | 625.9 | | | 177.4 | | 88.2 |
| ca rectum | 17 | 4 | 23.5 | 1.6 | 14.8 | 614.8 | | | 193 | | 85.5 |
| ca stomach | 47 | 19 | 40.4 | | 15.3 | 624.5 | | | 386.5 | | 84.3 |
| ca testis | 3 | 0 | 0.0 | | | | | | | | 85.9 |
| ca thyroid | 3 | 1 | 33.3 | | | | | | | | 87 |
| ca ukp | 7 | 6 | 85.7 | | 15.3 | 647 | | | 664 | | 84 |
| ca uterus | 6 | 3 | 50.0 | | 15.1 | | | | — | | 85.6 |
| ca? | 25 | 13 | 52.0 | 1.75 | 16.2 | 658 | | | 468 | | 88.8 |
| Carcinomatosis | 6 | 1 | 16.7 | | | 685 | | | | | 80.8 |
| Cardiac arrest | 8 | 2 | 25.0 | | 16.7 | — | | | — | | 95.5 |
| Cardiac dysthrythmia | 11 | 4 | 36.4 | | 15.9 | — | | | 281.7 | | 80.5 |
| Carotid stenosis | 3 | 2 | 66.7 | | — | | | | | | |
| Celiac | 103 | 6 | 5.8 | 1.8 | 16.3 | 556.7 | | | 300 | | 84.8 |
| Cerebro-vascular accident | 35 | 21 | 60.0 | 1.9 | 15.9 | 634 | | | 244 | | 91.4 |
| Cholecystectomy | 9 | 3 | 33.3 | | | | | | 202 | | 93 |
| Cholecystitis | 9 | 1 | 11.1 | 1.6 | 15.7 | 560 | | | 490 | | 90.6 |
| Chronic obstructive airway | 101 | 19 | 18.8 | | 15.4 | 580 | | | 322 | | 90.4 |
| disease | | | | | | | | | | | |
| Cirrhosis all | 30 | 10 | 33.3 | 1.7 | 1.7 | 566 | | | 141 | | 95.7 |
| Claudication | 5 | 2 | 40.0 | | 15.6 | 596 | | | 425 | | 88.2 |
| Coagulopathy | 3 | 2 | 66.7 | | 16.5 | 453 | | | 109 | | 97 |
| Congestive heart failure | 43 | 20 | 46.5 | | 15.6 | 538 | | | 256.3 | | 90.5 |
| Control | 216 | 11 | 5.1 | 1.8 | 15.9 | 657 | | | 234 | | 90.3 |
| Control women | 6 | 0 | 0.0 | 1.8 | 1 | | | | | | |
| Cord | 17 | 0 | 0.0 | | | | | | | | |
| Coronary artery disease | 48 | 15 | 31.3 | | 15.5 | 588.1 | | | 261.3 | | 86.1 |
| Crohns | 23 | 3 | 13.0 | | 13.8 | 661 | | | 339 | | 92.5 |
| Cushings + thal maj | 3 | 0 | 0.0 | | 13.3 | — | | | — | | 99 |
| Cystic fibrosis | 103 | 5 | 4.9 | 1.7 | | 453 | | | 253 | | 92.1 |
| Cystic fibrosis hz | 10 | 2 | 20.0 | 1.7 | | | | | | | |
| Cystic fibrosis mec ileus | 1 | 1 | 100.0 | | | | | | 373 | | 101 |
| D & C | 9 | 2 | 22.2 | | | | | | | | 85 |
| Deep vein thrombosis | 17 | 3 | 17.6 | | 14.4 | 659 | | | 463 | | 89.1 |
| Dehydration | 12 | 5 | 41.7 | | 14.5 | 524 | | | 257 | | 93.5 |
| Dementia | 5 | 4 | 80.0 | | 16.5 | 683 | | | 214 | | 84.8 |
| Diabetes mellitus | 14 | 6 | 42.9 | 1.7 | 15.5 | 557.5 | | | 222 | | 90.4 |
| Diaphragmatic hernia | 5 | 0 | 0.0 | | | | | | 499 | | 97.9 |
| Disc lesion | 15 | 2 | 13.3 | 1.6 | 16.1 | 560 | | | 233 | | 88.5 |
| Down's syndrome | 3 | 0 | 0.0 | | 16.6 | 591 | | | 107 | | 96.6 |
| Duodenal ulcer | 10 | 3 | 30.0 | | 15.2 | 589.1 | | | | | 88.7 |
| Dysphagia | 3 | 1 | 33.3 | | | 578 | | | 157 | | 88.7 |
| Dyspnoea | 115 | 14 | 12.2 | | 15.2 | 555 | 7.7 | 10.7 | 282.6 | | 66.9 |
| Emphesema | 11 | 2 | 18.2 | | 15.1 | 577 | | | 376 | | 88.3 |
| Epilepsy | 25 | 4 | 16.0 | 1.6 | 16.2 | 668 | | | 275 | | 91.7 |
| Esophagitis | 3 | 1 | 33.3 | | | | | | 228 | | 83 |
| Femoral popliteal bypass | 11 | 6 | 54.5 | | 15.1 | 643.3 | | | | | 87.6 |
| Fibroadenoma breast | 3 | 0 | 0.0 | | 14.9 | 375 | | | 182 | | 90.3 |
| Fractures | 64 | 15 | 23.4 | | 15.7 | 556 | | | 305 | | 89.5 |
| Gall stones | 14 | 3 | 21.4 | | | 626 | | | 286 | | 89.2 |
| Gangrene | 4 | 1 | 25.0 | 1.79 | 14.5 | 544 | | | 178 | | 86.5 |
| Gastric ulcer | 6 | 2 | 33.3 | | 15.6 | 523 | | | 253 | | 85 |
| Gastro-intestinal bleed | 55 | 18 | 32.7 | 1.8 | 15.6 | 502.4 | | | 250 | | 86 |
| Glandular fever | 5 | 2 | 40.0 | | | 718 | | | 248 | | 86.4 |
| Guillain Barre | 3 | 0 | 0.0 | | | | | | | | 89 |
| Hb AC | 2 | 0 | 0.0 | | | | | | | | 66 |
| Hb AE etc | 8 | 0 | 0.0 | | | | | | | | |
| Hb Agononi | 1 | 0 | 0.0 | | 18.4 | | | | | | |
| hb CC | 16 | 0 | 0.0 | 1.7 | 18 | — | | | — | | |
| Hb H disease | 2 | 0 | 0.0 | 1.96 | | | | | | | |
| hb S b thal | 4 | 1 | 25.0 | 1.6 | | 405 | | | 233 | | 78.3 |
| Hb SC | 7 | 0 | 0.0 | 1.7 | 17.6 | | | | | | 96 |
| hb SS all | 108 | 4 | 3.7 | | | 337 | | | 413 | | 92.1 |
| hb ss crisis | 20 | 2 | 10.0 | 1.7 | 16.1 | 323 | | | 344.6 | | 86 |
| hb ss no crisis | 81 | 2 | 2.5 | 1.7 | 15.5 | 352 | | | | | |
| Hb th maj or int never tx | 1 | 0 | 0.0 | 1.98 | | | | | | | |
| Hb thal E various | 7 | 0 | 0.0 | | | | | | 280 | | 86 |
| hb thal I txed | 2 | 0 | 0.0 | 1.65 | | 426 | | | 1002 | | 84.6 |
| Hb thal int | 12 | 0 | 0.0 | 1.6 | | 359.5 | | | 933.7 | | 80.4 |
| hb thal maj ALL | 75 | 3 | 4.0 | 1.7 | 14.1 | 449 | 8 | 7 | 387 | | 84.7 |
| hb thal maj never tx | 2 | 0 | 0.0 | 1.6 | | | | | 191 | | 95.7 |
| hb thal maj pre tx | 4 | 0 | 0.0 | 1.7 | | | | | | | |
| Hb thal major txed | 15 | 0 | 0.0 | 1.7 | | | | | 233 | | 88.8 |
| Hematuria | 10 | 5 | 50.0 | | 16.1 | 792 | | | | | |
| Hemoglobin AS | 9 | 1 | 11.1 | 1.8 | | 496 | | | 382 | | 85.3 |
| Hemolytic uremic syndrome | 2 | 1 | 50.0 | | 16.9 | | | | 92.7 | | 89.9 |
| Hepatitis | 11 | 2 | 18.2 | 1.62 | 16.7 | 555 | 11.5 | 17.5 | 252 | | 98.4 |
| Hereditary spherocytosiss | 17 | 3 | 17.6 | 1.5 | 15.4 | 610 | 7 | 13 | 364.5 | | 88.9 |
| Hereditary telangiectasia | 3 | 1 | 33.3 | | 14.4 | 446.2 | 10.7 | 20.5 | 261 | | 90.3 |
| Hernia | 17 | 8 | 47.1 | | 15.8 | 667 | | | 154 | | 100 |
| Herpes simplex | 1 | 0 | 0.0 | | | | | | 373 | | 92 |
| Herpes zoster | 5 | 1 | 20.0 | | 15.5 | 574.3 | 10 | 15 | 232 | | 95 |
| Herpes zoster | 4 | 0 | 0.0 | | 15.5 | 534 | | | | | |
| Hgb alpha 1 thal | 3 | 0 | 0.0 | | | | | | | | 70 |
| Hgb thal E hz | 4 | 0 | 0.0 | | | | | | | | |
| Hip replacement | 7 | 5 | 71.4 | | 16.2 | 477 | | | 161 | | 85 |
| Hiv | 8 | 5 | 62.5 | | 15.8 | 466 | | | 342 | | 81 |
| Hurlers | 1 | 0 | 0.0 | | | | | | | | 88 |
| Hyaline membrane disease | 4 | 0 | 0.0 | | | — | | | — | | 97.5 |
| Hydrocephalus | 10 | 3 | 30.0 | | 16.2 | 580 | | | 244 | | 87.6 |
| Hypersplenism | 7 | 1 | 14.3 | 1.8 | | 438 | | | 934 | | |
| Hypertension | 21 | 3 | 14.3 | | 15 | | | | | | |
| Hypertension malignant | 25 | 0 | 0.0 | 1.7 | | | | | 259 | | 85.4 |
| Hypotension | 2 | 0 | 0.0 | | 16.2 | | 11 | 14 | | | 65 |
| Hysterectomy | 14 | 4 | 28.6 | 1.75 | 16.3 | 525 | | | | | |
| Idiopathic thrombocytopenic | 39 | 4 | 10.3 | 1.7 | 15.6 | 489 | 9.3 | 15.9 | 71 | | 88.8 |
| purpura | | | | | | | | | | | |
| Infected | 5 | 1 | 20.0 | | 15.9 | 669 | | | 158 | | 99.2 |
| Interstitial lung disease | 3 | 0 | 0.0 | | 15.8 | | | | | | |
| Intest obstructn | 12 | 3 | 25.0 | | 15.6 | | 11 | 13.6 | | | |
| Ischemic bowel disease | 4 | 1 | 25.0 | | | — | | | | | 93.7 |
| Jaundice | 9 | 2 | 22.2 | | 15.2 | 609 | 11.2 | 13.9 | 350 | | 93.3 |
| Laminectomy | 6 | 0 | 0.0 | | 13.8 | | 12 | 16.5 | | | |
| Leukemia acute | 5 | 2 | 40.0 | | 17.5 | | 13 | 25.5 | | | 104 |
| Leukemia acute myeloid | 25 | 15 | 60.0 | | | 525 | | | 52 | | 84.7 |
| Leukemia ALL | 2 | 0 | 0.0 | | | | 9 | 8 | 220 | | 89.5 |
| Leukemia AML | 25 | 15 | 60.0 | 1.6 | 15.8 | 525 | 8 | 13 | 125 | | 84.7 |
| Leukemia CGL | 5 | 2 | 40.0 | 1.6 | | | 13 | 25.5 | 444 | | 93.9 |
| Leukemia CLL | 13 | 5 | 38.5 | | 16.6 | | | | 227.7 | | 89.7 |
| Leukemia CML | 2 | 1 | 50.0 | | | 451 | | | 70 | | 88.7 |
| Leukemia nos | 11 | 3 | 27.3 | | 15.9 | 485 | 9 | 7 | 50 | | 90 |
| Leukemia total | 71 | 34 | 47.9 | 1.6 | 16.2 | 557 | 10.5 | 16.8 | 230 | | 89.1 |
| Liver failure | 25 | 10 | 40.0 | 1.57 | 17 | 556 | 13.1 | 16.3 | 210 | | 97.1 |
| Lobectomy | 2 | 0 | 0.0 | 1.7 | 13.7 | | | | 387 | | 78.6 |
| Lung lesion/nodule | 29 | 5 | 17.2 | | 15.7 | 485 | | | 172 | | 81.5 |
| Lung tx | 4 | 1 | 25.0 | | 15.1 | | | | 314 | | 89.9 |
| Lver transplant | 9 | 1 | 11.1 | | 15.6 | | | | 373 | | 101 |
| Lymphoma H | 20 | 6 | 30.0 | | 14.4 | 533 | | | — | | — |
| Lymphoma NH | 36 | 12 | 33.3 | 1.6 | 15.3 | 957 | | | — | | 90 |
| Malaise | 17 | 2 | 11.8 | | | | | | 248 | | 85.5 |
| Malaria | 11 | 0 | 0.0 | | | 426 | | | 261 | | 102 |
| Meconium ileus (cystic | 1 | 1 | 100.0 | | | — | 10.8 | 13.3 | 460 | | 101 |
| fibrosis) | | | | | | | | | | | |
| Menorrhagia | 47 | 1 | 2.1 | 1.7 | 16.1 | 419.5 | | | — | | — |
| Mitral valve disease | 17 | 2 | 11.8 | | 14.7 | saml | 11 | 12 | 308 | | 85.5 |
| Mitral valve disease | 33 | 4 | 12.1 | | 15.6 | 479 | | | 26 | | 102 |
| Motor neurone disease | 3 | 3 | 100.0 | | 16.3 | — | | | | | 89 |
| mult myeloma | 4 | 0 | 0.0 | | | — | | | 250 | | 91.5 |
| mult myeloma | 14 | 17 | 121.4 | | 15.8 | 476 | | | 206 | | 94.3 |
| Multiple sclerosis | 8 | 2 | 25.0 | | 14.9 | — | | | 326 | | 91 |
| Muscular dystrophy all | 112 | 11 | 9.8 | 1.7 | 15 | 583 | 10.6 | 13.3 | 205 | | 91.4 |
| Muscular dystrophy beckers | 7 | 1 | 14.3 | | 13 | 645 | 9.5 | 15 | 260 | | 93 |
| Muscular dystrophy duchenne | 4 | 2 | 50.0 | | 15.5 | | 10.5 | 12.5 | | | |
| Muscular dystrophy nos | 65 | 4 | 6.2 | | 15.2 | 132 | 10.7 | 13.6 | 279 | | 87 |
| Muscular dystrophy sma | 4 | 2 | 50.0 | | 15.6 | | 10.3 | 12 | | | |
| Muscular dystrophy; | 20 | 1 | 5.0 | 1.7 | 14.9 | | 11 | 13 | | | |
| myotonic | | | | | | | | | | | |
| Myasthenia gravis | 4 | 1 | 25.0 | | | 471 | 11 | 13 | 243.7 | | 87.7 |
| Myelodysplasia | 115 | 5 | 4.3 | | | 370 | 12 | 16.7 | 295.2 | | 90.1 |
| Myelofibrosis | 24 | 11 | 45.8 | | 16.2 | 518 | 8 | 10 | 123 | | 82.8 |
| Myocardial infarct | 18 | 7 | 38.9 | | 16.4 | 693 | 10.5 | 13.2 | 245 | | 89.1 |
| Neonatal | 12 | 1 | 8.3 | | | — | 12 | 15 | 246 | 8 | 90.1 |
| Neoplasm benign | 13 | 0 | 0.0 | | | 630 | 9.5 | 19 | 339 | 14.8 | 81.8 |
| Neoplasm glioblastoma | 6 | 1 | 16.7 | | 16.3 | | | | — | | |
| New born | 110 | 1 | 0.9 | | | 573 | | | 383 | | 82.7 |
| Osteoarthritis | 28 | 6 | 21.4 | 1.6 | 15.2 | 619 | | | | | 80 |
| ovarian cyst | 6 | 1 | 16.7 | | 16.3 | 669 | | | 275 | | 90.7 |
| Pancreatitis | 13 | 1 | 7.7 | 1.76 | 16.2 | 566 | | | 234.4 | | 81.4 |
| Pancytopenia | 3 | 1 | 33.3 | | 16.8 | 344.6 | | | 283 | | 84.7 |
| Parkinsons | 2 | 0 | 0.0 | | 14.7 | 688 | | | 292.6 | | 88 |
| Peripheral vascular disease | 21 | 12 | 57.1 | | 15.4 | 562 | | | 235 | | 85.8 |
| Pernicious anemia | 6 | 0 | 0.0 | 1.9 | 15.3 | 547 | 10 | 12 | 298.4 | | 92.4 |
| Platelets giant | 3 | 1 | 33.3 | | 17.4 | 627 | | | 296 | | 85.7 |
| Platelets small | 9 | 1 | 11.1 | | 16.7 | 635 | | | 284 | | 85 |
| PN- | 41 | 21 | 51.2 | | 15.2 | 502.6 | | | 274 | | 85 |
| Polycythemia vera | 50 | 17 | 34.0 | 1.5 | 15.1 | 779.4 | | | 173 | | 92.1 |
| Polymyalgia | 3 | 1 | 33.3 | | | | | | 277.9 | | 85.1 |
| Polyneuropathy | 2 | 2 | 100.0 | | 14.6 | 567 | | | 312 | | 88.3 |
| Pregnancy 0 | 68 | 2 | 2.9 | | 14.4 | 432 | | | 422 | | 91.8 |
| Pregnancy 1 | 19 | 0 | 0.0 | | 15.5 | 411 | 12 | 18 | 323 | | 83 |
| Pregnancy 2 | 14 | 2 | 14.3 | 1.7 | | 397 | 10.5 | 14 | 294 | | 85 |
| Pregnancy 3 | 65 | 3 | 4.6 | 1.7 | | 421 | | | 298 | | 87 |
| Pregnancy 4 | 13 | 0 | 0.0 | 1.68 | | 433 | | | 554 | | 82.4 |
| Pregnancy 5-7 | 14 | 0 | 0.0 | 1.76 | 14.5 | 424 | | | 749 | | 81.1 |
| Pregnancy 8, 9 | 4 | 0 | 0.0 | | | 401.2 | | | 198.7 | | 98.3 |
| Pregnancy an nos | 53 | 1 | 1.9 | 1.7 | 15.7 | 465.2 | | | 244 | | 107.9 |
| Pregnancy L | 50 | 0 | 0.0 | 1.7 | 15.7 | 430 | | | | | |
| Pregnancy pn nos | 47 | 2 | 4.3 | 1.7 | 13.4 | | | | 274.3 | | 92.9 |
| Pregnancyan 10-20/40 | 10 | 1 | 10.0 | 1.7 | 15.64 | 429 | | | 249 | | 86.3 |
| Pregnancyan 20-29/40 | 15 | 0 | 0.0 | 1.76 | 14.3 | 464 | 11.5 | 15.3 | 246 | | 91.2 |
| Pregnancyan 21/40 | 5 | 0 | 0.0 | | | 453 | | | 299 | | 88.7 |
| Pregnancyan 30-34/40 | 18 | 0 | 0.0 | 1.68 | 15.3 | 430 | 12 | 16 | | | |
| Pregnancyan 32/40 | 18 | 0 | | 1.8 | 15.3 | 431 | | | 174 | | 92.1 |
| Pregnancyan 35-36/40 | 24 | 2 | 8.3 | 1.7 | 11.4 | 438 | 11 | 12 | | | |
| Pregnancyan 37-39/40 | 33 | 0 | 0.0 | 1.7 | | 430.3 | 11 | 12 | 388 | | 91.9 |
| Pregnancyan 40+ | 7 | 0 | 0.0 | 1.7 | | 456 | | | — | | — |
| Pregnancyan 40 n bp | 23 | 0 | 0.0 | 1.7 | 15.1 | 438 | | | 322 | | 85.6 |
| Pregnancyan 40-42/40 + | 30 | | 0.0 | 1.7 | 15 | 457.8 | | | | | |
| BP | | | | | | | | | | | |
| Pulmonary embolus not on | 12 | 1 | 8.3 | | 16 | 657 | | | 291 | | 86.3 |
| warf | | | | | | | | | | | |
| Pulmonary embolus on | 4 | 1 | 25.0 | | | | | | | | |
| warfarin | | | | | | | | | | | |
| Pulmonary fibrosis | 4 | 0 | 0.0 | | 15.4 | | | | 277 | | 90.3 |
| Pulmonary hypetension | 5 | 0 | 0.0 | | 15.1 | | 10 | 15.5 | 151 | | 95.5 |
| Pyloric stenosis | 5 | 1 | 20.0 | — | — | 556 | 10 | 15 | 240 | | 85.1 |
| Pyrexia of unknown origin | 16 | 6 | 37.5 | 15.6 | — | 476 | | | 205 | | 90.3 |
| Quadriplegia | 5 | 0 | 0.0 | 15 | 66.3 | — | | | | | 97 |
| Reiters | 2 | 1 | 50.0 | | 15 | | 11 | 13 | — | | 87 |
| Renal failure chronic | 275 | 135 | 49.1 | 1.6 | 16.2 | 542 | | | 221 | | 84.1 |
| Renal failure: acute | 9 | 2 | | 1.72 | 15.9 | 590.1 | | | 257.3 | | 83.6 |
| Renal stone | 13 | 3 | 23.1 | — | 15.5 | 687 | 12.5 | 11 | | | |
| Renal transplant | 19 | 9 | 47.4 | 1.7 | 14 | 388.5 | 10.9 | 14 | 339 | | 88.3 |
| Respiratory distress syndrome | 4 | 1 | 25.0 | — | — | — | 9.9 | 25.6 | 97 | | 93.1 |
| Respiratory failure | 5 | 4 | 80.0 | — | 15.25 | 530 | | | | | |
| Rheumatoid arthritis pen | 18 | 1 | 5.6 | — | — | — | | | 30 | | 90.3 |
| Rheumatoid arthritis all | 63 | 5 | 7.9 | | 14.2 | 659 | | | 456 | | 83.7 |
| Rheumatoid arthritis au | 9 | 2 | 22.2 | | | 346 | | | 249 | | |
| Rheumatoid arthritis az | 3 | 0 | 0.0 | | | | 9 | 8 | — | | 85 |
| Rheumatoid arthritis st | 3 | 0 | 0.0 | — | — | — | | | 167 | | 88.2 |
| RT | 10 | 4 | 40.0 | 1.7 | 14.7 | 448 | | | | | 76 |
| Sarcoid | 17 | 0 | 0.0 | — | 15.5 | 677 | | | 263 | | 93 |
| Sarcoma | 5 | 3 | 60.0 | | 15 | — | | | 78.7 | | 74 |
| Satelitism | 1 | 0 | 0.0 | | 14.6 | 535 | | | | | 92 |
| Scleroderma | 6 | 0 | 0.0 | — | 15.1 | — | 10.7 | 11.2 | | | |
| Scoliosis | 4 | 0 | 0.0 | — | 16.2 | 425 | 11 | 17 | | | 87.3 |
| Sepsis | 8 | 6 | 75.0 | 1.73 | 15.6 | 376 | | | — | | 84.8 |
| Sleep apnoea | 5 | 0 | 0.0 | | 15.8 | | | | | | |
| Spina bifida | 5 | 0 | 0.0 | | 13.8 | | | | | | |
| Splenectomy | 8 | 1 | 12.5 | | 15.9 | 551 | | | | | |
| Sprue | 1 | 1 | 100.0 | | | 562 | | | 379 | | 84.9 |
| Stem cells | 4 | 0 | 0.0 | | 15.6 | — | | | | | |
| Subacute bacterial | 9 | 2 | 22.2 | 1.7 | — | 619.5 | | | | | |
| endocarditis | | | | | | | | | | | |
| Syncope | 5 | 0 | 0.0 | | 16.6 | | | | | | |
| Systemic lupus | 7 | 0 | 0.0 | — | 15.2 | — | | | | | |
| erythethematosis | | | | | | | | | | | |
| T's & As | 6 | 0 | 0.0 | | 16.8 | | | | | | 87 |
| Temperal arteritis | 4 | 3 | 75.0 | | | 368 | | | | | |
| Thalassemia beta trait | 81 | 1 | 1.2 | 1.8 | 17.5 | 520 | 7.7 | 10.7 | 282.6 | | 88.7 |
| Thrombocytopenia | 7 | 1 | 14.3 | 1.6 | 16.7 | 569 | 11 | 19 | 160.5 | | 90.6 |
| Thrombocytosis | 5 | 0 | 0.0 | | 14 | 506.2 | 9 | 9 | 623.5 | | 81.4 |
| Thrombotic | 2 | 0 | 0.0 | | 16.6 | 456 | | | 199 | | 94.8 |
| thrombocytopenic purpura | | | | | | | | | | | |
| Thyrotoxicosis | 7 | 0 | 0.0 | 1.74 | 16.4 | 442 | 10.5 | 14.5 | 183 | | 84.9 |
| Transient ischemic attacks | 7 | 3 | 42.9 | | 16.2 | 661 | 12 | 15 | 195 | | 93.3 |
| Turp | 17 | 14 | 82.4 | 1.8 | 16.7 | 659 | | | 376 | | 87.4 |
| Ulcerative colitis | 35 | 4 | 11.4 | | 16.1 | 585 | 9.5 | 11.5 | 408 | | 83.7 |
| Urinary retention | 8 | 5 | 62.5 | | 15.8 | 545 | | | 227 | | 97 |
| Uterine fibroids | 22 | 1 | 4.5 | | 15.7 | 634 | 10.3 | 14.2 | 218 | | 90.1 |
| Ventric tachy | 12 | 3 | 25.0 | | 15.6 | 618 | 11.8 | 13.8 | 305 | | 97.6 |
| Volvulus sigmoid | 3 | 1 | 33.3 | | | | | | | | 84 |
| Waldenstrom's | 1 | 0 | 0.0 | | 15 | 444 | | | 221 | | 87 |
| macroglobulinemia | | | | | | | | | | | |

| | | | | | | W10% | | uniformity | uniformity | | | GHOST |
|---|---|---|---|---|---|---|---|---|---|---|---|---|
| | | N | DEATH | | | avg cell | | cell by | spherical | | | GAP |
| DIAGNOSIS | N | (died) | % | Hgb | MCH | uniformity | | cell scatter | SD | FRAGS | | GG |
| Abdominal aortic | 19 | 8 | 42.1 | | | | | | | | | |
| aneurysm | | | | | | | | | | | | |
| AIDS | 4 | 2 | 50.0 | | | | | 3.7 | | 0.5 | | 0.5 |
| Alcohol xs | 30 | 10 | 33.3 | 13 | 31.8 | 26 | | 3.9 | | 0.5 | | 1 |
| Alpha 1 antitrypsin | 3 | 0 | 0.0 | | | | | | | | | |
| deficiency | | | | | | | | | | | | |
| Amyloid | 3 | 1 | 33.3 | 8.9 | 25.5 | 22 | | | | | | |
| Anemia all | 387 | 100 | 25.8 | | | 24.9 | | 4.2 | | 0.4 | | |
| Anemia aplastic | 9 | 4 | 44.4 | 9.8 | 29.6 | | | 4.7 | | 0 | | 1.1 |
| Anemia elliptocytosis | 5 | 0 | 0.0 | 10.7 | 26 | 23 | | 6 | | 0 | | 1 |
| Anemia Fe diff | 103 | 30 | 29.1 | 9.5 | 23.3 | 24 | | 4.8 | | 0 | | 1 |
| Anemia | 3 | 1 | 33.3 | 13.2 | 25.3 | 19 | | | | | | |
| megaloblastic | | | | | | | | | | | | |
| Anemia | 2 | 1 | 50.0 | | | | | | | | | |
| microangiopathetic | | | | | | | | | | | | |
| Anemia sideroblastic | 8 | 4 | 50.0 | 9.2 | 30.5 | 23 | | 4 | | 0 | | 0.25 |
| Anemia, hemolytic | 30 | 6 | 20.0 | 11.6 | 31.2 | 19 | | 3.8 | | 5 | | |
| Anemia, hemolytic: | 5 | 0 | 0.0 | 14.2 | 33.4 | 17 | | | | | | |
| g6pd | | | | | | | | | | | | |
| Anemia, hemolytic: | 4 | 1 | 25.0 | 13 | 32.1 | | | 3.8 | | 1 | | 0.5 |
| AIHA | | | | | | | | | | | | |
| Angina stable | 26 | 6 | 23.1 | 13.4 | 29.3 | 21 | | 4 | | 1 | | 1.7 |
| Angina unstable | 38 | 17 | | 12.7 | 29.3 | | | 3.8 | | 0.5 | | 1.3 |
| Aortic stenosis | 9 | 3 | 33.3 | 9.9 | 35.9 | | | 4.3 | | 1.5 | | 1.6 |
| Aortic valve disease | 19 | 6 | | 12.7 | 29.7 | 20.3 | | 4.4 | | 1.5 | | 0.9 |
| all other | | | | | | | | | | | | |
| Aortic valve repair | 12 | 5 | 41.7 | 12.7 | 29.7 | 20.3 | | 4.5 | | 1.4 | | 1 |
| Aorto femoral bypass | 4 | 1 | | 13.2 | 31.5 | 23 | | | | | | |
| Appendicitis acute | 8 | 2 | 25.0 | 14.1 | 28.7 | 32 | | 3.5 | | 0 | | 1.5 |
| Arteriovenous | 2 | 0 | | | | | | 4.25 | | 0.35 | | 1.75 |
| malformation | | | | | | | | | | | | |
| Arthritis | 14 | 2 | | 13.9 | 29.2 | 31 | | | | | | |
| Artrioseptal defect | 3 | 0 | 0.0 | | | | | 3.75 | | 0.75 | | 1.75 |
| Artrioseptal defect | 1 | 0 | | | | 24 | | | | | | |
| post repair | | | | | | | | | | | | |
| Asbestosis | 2 | 0 | 0.0 | | | | | 4.5 | | 0.25 | | 2.2 |
| ASD | 8 | 0 | | 13.3 | 30.7 | | | 4.25 | | 0.5 | | 1.67 |
| Asthma | 62 | 4 | 6.5 | 13.6 | 30.6 | 18 | | 4.3 | | 1 | | 1.8 |
| Ataxia | 8 | 4 | 50.0 | 13.8 | 29.4 | — | | 4.3 | | 0.6 | | 1.6 |
| Atrial fibrillation | 18 | 9 | 50.0 | 15.6 | 30.3 | | | 3.7 | | F = 0.8 | | 1.1 gg |
| Atrial flutter | 5 | 1 | 20.0 | 12.2 | 28.4 | 22 | | 3.8 | | 1.3 | | 1.2 |
| AUO | 44 | 13 | 29.5 | 16 | 31.3 | — | | 4.5 | | 0.5 | | 1 |
| Avascular necrosis | 3 | 0 | 0.0 | | | | | | | | | |
| Benign prostatic | 4 | 2 | 50.0 | 15.7 | 31.3 | | | 3.3 | | 0.5 | | 1.5 |
| hypertrophy | | | | | | | | | | | | |
| Bile duct obstruction | 4 | 2 | 50.0 | 11.5 | 23.5 | | | | | | | |
| Bladder stone | 2 | 1 | 50.0 | 14.5 | 28.5 | — | | 3.5 | | 0.6 | | 1 |
| Bladder tumor | 6 | 4 | 66.7 | 11.7 | 28.1 | | | 3.9 | | 0.4 | | 1.25 |
| Bleeding pr | 10 | 5 | 50.0 | 12.1 | 28.3 | 28.5 | | | | | | |
| Blood donors | 902 | 1 | 0.1 | 11.6 | 28.1 | | | | | | | |
| Brain tumor | 47 | 7 | 14.9 | 14 | 31.6 | 27 | | 3.9 | | 1.2 | | 1.4 |
| Bronchiectasis | 5 | 1 | 20.0 | | | | | 4.4 | | 1 | | 1.4 |
| Bronchitis | 10 | 2 | 20.0 | | | 24 | | 4.5 | | 1 | | 1.7 |
| Burns major | 1 | 0 | 0.0 | | | | | 3 | | 0.5 | | 1 |
| ca basal cell | 3 | 0 | 0.0 | | | | | 3 | | 0.5 | | 1 |
| carcinoma | | | | | | | | | | | | |
| ca bladder | 24 | 13 | 54.2 | 11.9 | 26.7 | 28.3 | | 4.4 | | 1.5 | | 1.5 |
| ca breast | 43 | 14 | 32.6 | 13.1 | 29.4 | 24 | | 4 | | 0.5 | | 1.8 |
| ca bronchus | 49 | 20 | 40.8 | 13.8 | 28.3 | 24.3 | | 4.5 | | 0 | | |
| ca carcinoid | 8 | 4 | 50.0 | 11.3 | 26.1 | 21.6 | | | | | | |
| ca colon | 162 | 31 | 19.1 | 11.8 | 27.1 | 23 | | 4.2 | | 5 | | 2.1 |
| ca common bile duct | 6 | 4 | 66.7 | 12.7 | 31.3 | 26 | | | | | | |
| ca kidney | 6 | 4 | 66.7 | 10.4 | 25 | 22 | | 3.5 | | 0.5 | | 1 |
| ca lung | 51 | 17 | 33.3 | 13 | 26.4 | | | 4.2 | | 5 | | 1.9 |
| ca malignant | 13 | 5 | 38.5 | 13 | 30.8 | 22 | | 3.7 | | 0.5 | | 1.4 |
| melanoma | | | | | | | | | | | | |
| ca nos | 13 | 4 | 30.8 | 11.2 | 24 | 31 | | 4.2 | | 1 | | 2 |
| ca oes | 16 | 7 | 43.8 | 11.7 | 29.3 | 18 | | 4.3 | | 1.8 | | 2.2 |
| ca ovary | 25 | 13 | 52.0 | 10.9 | 28.2 | 21.9 | | 4.1 | | 0.9 | | 2 |
| ca pancreas | 10 | 7 | 70.0 | 10.6 | 24.1 | 22.5 | | 4 | | 0.2 | | 2 |
| ca prostate | 32 | 12 | 37.5 | 12.5 | 28.9 | 26 | | 4 | | 0.3 | | 1.7 |
| ca rectum | 17 | 4 | 23.5 | 12.7 | 28.5 | 22 | | 4 | | 0.2 | | 1 |
| ca stomach | 47 | 19 | 40.4 | 10.9 | 27.5 | 24 | | | | | | |
| ca testis | 3 | 0 | 0.0 | 13.6 | 25.4 | 24 | | | | | | |
| ca thyroid | 3 | 1 | 33.3 | 13.6 | 28.9 | 15 | | | | | | |
| ca ukp | 7 | 6 | 85.7 | 12.3 | 26.9 | 36.3 | | | | | | |
| ca uterus | 6 | 3 | 50.0 | 11.8 | 27.4 | 25 | | | | | | |
| ca? | 25 | 13 | 52.0 | 12.9 | 28.3 | 23.9 | | 3.9 | | 1.1 | | 1.9 |
| Carcinomatosis | 6 | 1 | 16.7 | 12.2 | 25.2 | | | | | | | |
| Cardiac arrest | 8 | 2 | 25.0 | 14.2 | 33.9 | 20 | | 3 | | 0 | | 0 |
| Cardiac dysthrythmia | 11 | 4 | 36.4 | 9.1 | 25.5 | — | | 4.2 | | 0.8 | | 1.3 |
| Carotid stenosis | 3 | 2 | 66.7 | | | | | | | | | |
| Celiac | 103 | 6 | 5.8 | 11.3 | 27.5 | 25 | | 5.4 | | 0 | | 1.2 |
| Cerebro-vascular | 35 | 21 | 60.0 | 14.1 | 31.2 | 24 | | 4 | | | | |
| accident | | | | | | | | | | | | |
| Cholecystectomy | 9 | 3 | 33.3 | 14.4 | 30.8 | 21 | | | | | | |
| Cholecystitis | 9 | 1 | 11.1 | 12.9 | 28.8 | 21.5 | | 4.8 | | 0.6 | | 1.25 |
| Chronic obstructive | 101 | 19 | 18.8 | 11.9 | 29.5 | 26.3 | | 4.2 | | 5 | | 1.5 |
| airway disease | | | | | | | | | | | | |
| Cirrhosis all | 30 | 10 | 33.3 | 12.5 | 30.7 | 26.1 | | 3.3 | | 1.5 | | 0.5 |
| Claudication | 5 | 2 | 40.0 | 13.6 | 29.1 | 30 | | 3.5 | | 1 | | 1.5 |
| Coagulopathy | 3 | 2 | 66.7 | 10.2 | 30.7 | | | 3.7 | | 0.4 | | 1.3 |
| Congestive heart | 43 | 20 | 46.5 | 12.6 | 29.3 | 21.3 | | 4 | | 1 | | 1.4 |
| failure | | | | | | | | | | | | |
| Control | 216 | 11 | 5.1 | 13.5 | 30.3 | 21.3 | | 4.6 | | 0.1 | | 2.1 |
| Control women | 6 | 0 | 0.0 | | | | | | | | | |
| Cord | 17 | 0 | 0.0 | | | | | | | | | |
| Coronary artery | 48 | 15 | 31.3 | 12.6 | 28.4 | 22.4 | | 4 | | 1.1 | | 1.6 |
| disease | | | | | | | | | | | | |
| Crohns | 23 | 3 | 13.0 | 12.6 | 29.9 | 20.7 | | | | | | |
| Cushings + thal maj | 3 | 0 | 0.0 | 18.3 | 32.7 | | | 4 | | 5 | | 2 |
| Cystic fibrosis | 103 | 5 | 4.9 | 13.8 | 29.6 | 26 | | 5 | | 0 | | 1.5 |
| Cystic fibrosis hz | 10 | 2 | 20.0 | | | | | | | | | |
| Cystic fibrosis mec | 1 | 1 | 100.0 | 11.6 | | 26 | | | | | | |
| ileus | | | | | | | | | | | | |
| D & C | 9 | 2 | 22.2 | 13.5 | 28.2 | 23 | | | | | | |
| Deep vein thrombosis | 17 | 3 | 17.6 | 13.5 | 29.2 | | | 4.5 | | 1.5 | | 1 |
| Dehydration | 12 | 5 | 41.7 | 11 | 30.1 | | | 3.8 | | 0.6 | | 1.6 |
| Dementia | 5 | 4 | 80.0 | 13.4 | 27.7 | 23 | | 3.5 | | 1 | | 2 |
| Diabetes mellitus | 14 | 6 | 42.9 | 14.1 | 30.5 | 23.2 | | 4 | | 0 | | 1 |
| Diaphragmatic hernia | 5 | 0 | 0.0 | 14.6 | 31.1 | 25.3 | | | | | | |
| Disc lesion | 15 | 2 | 13.3 | 12.5 | 29.2 | — | | 4.2 | | 0.6 | | 1.2 |
| Down's syndrome | 3 | 0 | 0.0 | 13 | 31.3 | 19 | | 4 | | 0.5 | | 1.5 |
| Duodenal ulcer | 10 | 3 | 30.0 | 11.3 | 27.8 | 30 | | | | | | |
| Dysphagia | 3 | 1 | 33.3 | 11.8 | 29 | | | 4.5 | | 0.6 | | 1.5 |
| Dyspnoea | 115 | 14 | 12.2 | 11.6 | 20.6 | 26 | | 5.4 | | 0.4 | | |
| Emphesema | 11 | 2 | 18.2 | 11.6 | 29 | | | 4 | | 1.4 | | 1.4 |
| Epilepsy | 25 | 4 | 16.0 | 13.2 | 31 | 26.2 | | 4.1 | | 1 | | 1.3 |
| Esophagitis | 3 | 1 | 33.3 | 12.4 | 27.6 | | | 4.75 | | 2.5 | | 2 |
| Femoral popliteal | 11 | 6 | 54.5 | 11.4 | 28.7 | 23.7 | | 4 | | | | 1.5 |
| bypass | | | | | | | | | | | | |
| Fibroadenoma breast | 3 | 0 | 0.0 | 10.9 | 30.1 | | | | | | | |
| Fractures | 64 | 15 | 23.4 | 11.9 | 29.2 | 23 | | 3.9 | | 0.8 | | 1.4 |
| Gall stones | 14 | 3 | 21.4 | 13.3 | 29.6 | 17 | | 3.9 | | 0.7 | | 1.3 |
| Gangrene | 4 | 1 | 25.0 | 8.8 | 27 | 35 | | 4.5 | | 2.3 | | 1.75 |
| Gastric ulcer | 6 | 2 | 33.3 | 12.3 | 27.5 | 17 | | 4 | | 0.3 | | 1.2 |
| Gastro-intestinal | 55 | 18 | 32.7 | 11 | 27.4 | 23 | | 3.9 | | 0.8 | | 1.3 |
| bleed | | | | | | | | | | | | |
| Glandular fever | 5 | 2 | 40.0 | 14.2 | 28.7 | | | 22 | | | | |
| Guillain Barre | 3 | 0 | 0.0 | 12.7 | 29.5 | | | 4 | | 0.3 | | 1.3 |
| Hb AC | 2 | 0 | 0.0 | 13.9 | 19 | | | | | | | |
| Hb AE etc | 8 | 0 | 0.0 | | | 33 | | | | | | |
| Hb Agononi | 1 | 0 | 0.0 | | | | | | 3 | | 1 | 0 |
| hb CC | 16 | 0 | 0.0 | | | | | 4.9 | | 1.5 | | 0.1 |
| Hb H disease | 2 | 0 | 0.0 | | | | | | | | | |
| hb S b thal | 4 | 1 | 25.0 | 10.5 | 25.1 | | | 4.1 | | 0 | | 0 |
| Hb SC | 7 | 0 | 0.0 | 13.5 | 29.3 | | | 3.9 | | 0.5 | | 1 |
| hb SS all | 108 | 4 | 3.7 | 8.4 | 30.7 | 27 | | 3.4 | | 3 | | |
| hb ss crisis | 20 | 2 | 10.0 | 9.8 | 28.9 | 30.3 | | 3.7 | | 2.5 | | 0.5 |
| hb ss no crisis | 81 | 2 | 2.5 | | | 33 | | | | | | |
| Hb th maj or int never | 1 | 0 | 0.0 | | | | | 3 | | | | 0 |
| tx | | | | | | | | | | | | |
| Hb thal E various | 7 | 0 | 0.0 | 13.9 | 29 | 21 | | | | | | |
| hb thal I txed | 2 | 0 | 0.0 | 10.65 | 31.5 | | | 4.5 | | 0 | | 0 |
| Hb thal int | 12 | 0 | 0.0 | 8.51 | 25 | 40 | | 3.7 | | 0 | | 0 |
| hb thal maj ALL | 75 | 3 | 4.0 | 11.4 | 27.8 | 28 | | 4.9 | | 0.5 | | 0.5 |
| hb thal maj never tx | 2 | 0 | 0.0 | 11.1 | 30.9 | | | 3.8 | | 0.8 | | 1.4 |
| hb thal maj pre tx | 4 | 0 | 0.0 | | | | | 4.5 | | 0 | | 0.5 |
| Hb thal major txed | 15 | 0 | 0.0 | 11.44 | 28.6 | 23 | | 4 | | 0.8 | | 1.5 |
| Hematuria | 10 | 5 | 50.0 | | | | | | | | | |
| Hemoglobin AS | 9 | 1 | 11.1 | 13.1 | 27.2 | | | 4.5 | | | | |
| Hemolytic uremic | 2 | 1 | 50.0 | 12.7 | 29.6 | 24 | | 4.4 | | 0.6 | | 1 |
| syndrome | | | | | | | | | | | | |
| Hepatitis | 11 | 2 | 18.2 | 13.3 | 32.4 | | | | | | | |
| Hereditary | 17 | 3 | 17.6 | 14.1 | 30.4 | 20.6 | | 4.4 | | 0.4 | | 1.1 |
| spherocytosiss | | | | | | | | | | | | |
| Hereditary | 3 | 1 | 33.3 | 11.1 | 30.1 | 20 | | 4 | | 1 | | 2 |
| telangiectasia | | | | | | | | | | | | |
| Hernia | 17 | 8 | 47.1 | 14.2 | 34.6 | 15 | | 3.7 | | 0 | | 0.5 |
| Herpes simplex | 1 | 0 | 0.0 | 13.7 | 29.8 | | | 3.8 | | 0.9 | | 1 |
| Herpes zoster | 5 | 1 | 20.0 | 13.5 | 32.8 | 20.3 | | 3.5 | | 0 | | 0.2 |
| Herpes zoster | 4 | 0 | 0.0 | | | | | | | | | |
| Hgb alpha 1 thal | 3 | 0 | 0.0 | 12.8 | 21.5 | 13 | | | | | | |
| Hb thal E hz | 4 | 0 | 0.0 | | | 13 | | | | | | |
| Hip replacement | 7 | 5 | 71.4 | | | | | | | | | |
| Hiv | 8 | 5 | 62.5 | 11.9 | 27 | 20 | | 3.8 | | 1.3 | | 1.8 |
| Hurlers | 1 | 0 | 0.0 | 13.6 | 29.1 | 22 | | 3.5 | | 0.5 | | 1 |
| Hyaline membrane | 4 | 0 | 0.0 | 12.8 | 32.5 | | | | | | | |
| disease | | | | | | | | | | | | |
| Hydrocephalus | 10 | 3 | 30.0 | 12.8 | 28.9 | 29.3 | | 3.5 | | 1 | | 0.5 |
| Hypersplenism | 7 | 1 | 14.3 | | | | | | | | | |
| Hypertension | 21 | 3 | 14.3 | | 19 | | | | | | | |
| Hypertension | 25 | 0 | 0.0 | 14.1 | 28.1 | | | 3.5 | | 0.2 | | 1 |
| malignant | | | | | | | | | | | | |
| Hypotension | 2 | 0 | 0.0 | 11.1 | 19.5 | | | | | | | |
| Hysterectomy | 14 | 4 | 28.6 | | | | | | | | | |
| Idiopathic | 39 | 4 | 10.3 | 13.2 | 28.2 | 30 | | 4.3 | | 0.75 | | 0.95 |
| thrombocytopenic | | | | | | | | | | | | |
| purpura | | | | | | | | | | | | |
| Infected | 5 | 1 | 20.0 | | | | | | | | | |
| Interstitial lung | 3 | 0 | 0.0 | | | | | | | | | |
| disease | | | | | | | | | | | | |
| Intest obstructn | 12 | 3 | 25.0 | | | | | 3.7 | | 0.8 | | 1.4 |
| Ischemic bowel | 4 | 1 | 25.0 | 12.2 | 30.8 | | | | | | | |
| disease | | | | | | | | | | | | |
| Jaundice | 9 | 2 | 22.2 | 11.1 | 33.5 | | | 4.4 | | 0.9 | | 1.6 |
| Laminectomy | 6 | 0 | 0.0 | | | | | 4 | | 1.5 | | 1.6 |
| Leukemia acute | 5 | 2 | 40.0 | 8.8 | 33.8 | | | 3.5 | | 0.2 | | 0.7 |
| Leukemia acute | 25 | 15 | 60.0 | 10.6 | 28.7 | 23 | | 3.8 | | 0.25 | | |
| myeloid | | | | | | | | | | | | |
| Leukemia ALL | 2 | 0 | 0.0 | 13 | 29.4 | 20 | | | | | | |
| Leukemia AML | 25 | 15 | 60.0 | 10.5 | 28.5 | 21 | | 3.8 | | 0.3 | | 1.3 |
| Leukemia CGL | 5 | 2 | 40.0 | 12.9 | 31 | 31.5 | | 3 | | 0 | | 0 |
| Leukemia CLL | 13 | 5 | 38.5 | 12.9 | 30 | 22.3 | | | | | | |
| Leukemia CML | 2 | 1 | 50.0 | 8.6 | 29 | 22 | | | | | | |
| Leukemia nos | 11 | 3 | 27.3 | 11 | 33.4 | 21 | | 4 | | 0 | | 1 |
| Leukemia total | 71 | 34 | 47.9 | 11.6 | 29.5 | 23.4 | | 3.5 | | 0.4 | | 1.5 |
| Liver failure | 25 | 10 | 40.0 | 13.1 | 32.4 | 22 | | 3 | | 1 | | 1.4 |
| Lobectomy | 2 | 0 | 0.0 | 11.3 | 24.7 | 23 | | 4.5 | | 0.4 | | 1 |
| Lung lesion/nodule | 29 | 5 | 17.2 | 10.6 | 28.4 | 26.5 | | 3.9 | | 0.9 | | 1 |
| Lung tx | 4 | 1 | 25.0 | 13.6 | 30 | 21.7 | | 4.8 | | 0.4 | | 1.8 |
| Lver transplant | 9 | 1 | 11.1 | 11.6 | — | | | | | | | |
| Lymphoma H | 20 | 6 | 30.0 | — | | | | 5 | | 0.2 | | 1 |
| Lymphoma NH | 36 | 12 | 33.3 | 15 | 31.4 | | | | | 3.9 | | 1.4 |
| Malaise | 17 | 2 | 11.8 | 13.7 | 28.9 | 20 | | | | | | |
| Malaria | 11 | 0 | 0.0 | 10 | 34 | | | 4.7 | | 0 | | 2 |
| Meconium ileus | 1 | 1 | 100.0 | 11.6 | | 26 | | | | | | |
| (cystic fibrosis) | | | | | | | | | | | | |
| Menorrhagia | 47 | 1 | 2.1 | — | — | | | | | | | |
| Mitral valve disease | 17 | 2 | 11.8 | 12.8 | 28.1 | 32 | | 4.1 | | 1.8 | | 1.5 |
| Mitral valve disease | 33 | 4 | 12.1 | 8 | 34 | | | 5 | | 0.5 | | 1.5 |
| Motor neurone | 3 | 3 | 100.0 | 11.2 | 29.5 | | | | | | | |
| disease | | | | | | | | | | | | |
| mult myeloma | 4 | 0 | 0.0 | 13.3 | 30.6 | 30 | | 3.4 | | 0.6 | | 1 |
| mult myeloma | 14 | 17 | 121.4 | 12.8 | 30.5 | | | 5 | | 0 | | 3 |
| Multiple sclerosis | 8 | 2 | 25.0 | 13 | 30 | 31 | | 3.5 | | 0.5 | | 1.5 |
| Muscular dystrophy | 112 | 11 | 9.8 | 11.9 | 30 | 30 | | 4.2 | | 0.8 | | 1.4 |
| all | | | | | | | | | | | | |
| Muscular dystrophy | 7 | 1 | 14.3 | 13.9 | 31.6 | | | 4.4 | | 1.4 | | 1.1 |
| beckers | | | | | | | | | | | | |
| Muscular dystrophy | 4 | 2 | 50.0 | | | | | 5 | | 0.7 | | 1.3 |
| duchenne | | | | | | | | | | | | |
| Muscular dystrophy | 65 | 4 | 6.2 | 12.5 | 30.4 | | | 4 | | 1 | | 1 |
| nos | | | | | | | | | | | | |
| Muscular dystrophy | 4 | 2 | 50.0 | | | | | 4.2 | | 1 | | 1 |
| sma | | | | | | | | | | | | |
| Muscular dystrophy; | 20 | 1 | 5.0 | | | | | 4 | | 1 | | 1 |
| myotonic | | | | | | | | | | | | |
| Myasthenia gravis | 4 | 1 | 25.0 | 17.8 | 30.2 | 24 | | 4.1 | | 0.5 | | 1.4 |
| Myelodysplasia | 115 | 5 | 4.3 | 13.4 | 29.6 | 20 | | 3.9 | | 1 | | 1.4 |
| Myelofibrosis | 24 | 11 | 45.8 | 9.9 | 26.3 | | | 3.5 | | 2 | | 0.5 |
| Myocardial infarct | 18 | 7 | 38.9 | 13.1 | 30.3 | 36.3 | | 3.6 | | 0.7 | | 1.1 |
| Neonatal | 12 | 1 | 8.3 | 14.1 | 29 | | | 4 | | 1 | | 2 |
| Neoplasm benign | 13 | 0 | 0.0 | 8.4 | 23.6 | | | 3.5 | | 1 | | 0.3 |
| Neoplasm | 6 | 1 | 16.7 | 16.5 | | | | 4.4 | | 0.9 | | 1.9 |
| glioblastoma | | | | | | | | | | | | |
| New born | 110 | 1 | 0.9 | 11.7 | 27.4 | 32.5 | | 3.8 | | 0.5 | | 0.9 |
| Osteoarthritis | 28 | 6 | 21.4 | 11.3 | 26.3 | | | | | | | |
| ovarian cyst | 6 | 1 | 16.7 | 12.5 | 30.6 | | | 5 | | 0 | | 1.3 |
| Pancreatitis | 13 | 1 | 7.7 | 10.3 | 27.4 | | | 4.8 | | 0 | | 0.7 |
| Pancytopenia | 3 | 1 | 33.3 | 10.9 | 27.4 | 17 | | 4.4 | | 0 | | 0.8 |
| Parkinsons | 2 | 0 | 0.0 | 11 | 29.4 | 22.5 | | 4.6 | | 0.2 | | 1.2 |
| Peripheral vascular | 21 | 12 | 57.1 | 11.3 | 28.3 | | | 4.7 | | 0 | | 1 |
| disease | | | | | | | | | | | | |
| Pernicious anemia | 6 | 0 | 0.0 | 11 | 30.8 | 17 | | 4.6 | | 0.2 | | 1 |
| Platelets giant | 3 | 1 | 33.3 | 11.4 | 28.1 | 18 | | 4.8 | | 0 | | 0.75 |
| Platelets small | 9 | 1 | 11.1 | 11.7 | 28 | | | 4.9 | | 0.5 | | 1 |
| PN- | 41 | 21 | 51.2 | 12 | 27.4 | | | 4.6 | | 0 | | 1.1 |
| Polycythemia vera | 50 | 17 | 34.0 | 11.5 | 30.7 | 20 | | 4.5 | | 0.3 | | 1.5 |
| Polymyalgia | 3 | 1 | 33.3 | 11.6 | 27.6 | | | | | | | |
| Polyneuropathy | 2 | 2 | 100.0 | 11.3 | 28.9 | | | 4.4 | | 0.5 | | 1.3 |
| Pregnancy 0 | 68 | 2 | 2.9 | 10.5 | 30 | | | 4.3 | | | | 2 |
| Pregnancy 1 | 19 | 0 | 0.0 | 11.4 | 27.9 | 11 | | 4.5 | | 0.1 | | 1.4 |
| Pregnancy 2 | 14 | 2 | 14.3 | | 28.2 | 19 | | 4.4 | | 0.4 | | 1.4 |
| Pregnancy 3 | 65 | 3 | 4.6 | 10.6 | 28 | 27 | | 4.7 | | 0.7 | | 0.9 |
| Pregnancy 4 | 13 | 0 | 0.0 | 11 | 25.4 | 17.5 | | | | | | |
| Pregnancy 5-7 | 14 | 0 | 0.0 | 12.4 | 26.9 | 26 | | | | | | |
| Pregnancy 8, 9 | 4 | 0 | 0.0 | 12.6 | 34.1 | 19.5 | | | | | | |
| Pregnancy an nos | 53 | 1 | 1.9 | 13.9 | 34.8 | 40 | | | | | | |
| Pregnancy L | 50 | 0 | 0.0 | | | | | 4.5 | | 5 | | 1 |
| Pregnancy pn nos | 47 | 2 | 4.3 | 15.3 | 31.3 | | | 4.1 | | 0.9 | | 1.8 |
| Pregnancyan 10- | 10 | 1 | 10.0 | 12.3 | 27.7 | 28 | | | | | | |
| 20/40 | | | | | | | | | | | | |
| Pregnancyan 20- | 15 | 0 | 0.0 | 11.3 | 26 | | | 4.4 | | 0 | | 1.1 |
| 29/40 | | | | | | | | | | | | |
| Pregnancyan 21/40 | 5 | 0 | 0.0 | 10.8 | 28.8 | 23 | | 4 | | 0 | | 1 |
| Pregnancyan 30- | 18 | 0 | 0.0 | | | | | 4.2 | | 0.75 | | 1.8 |
| 34/40 | | | | | | | | | | | | |
| Pregnancyan 32/40 | 18 | 0 | | 11.5 | 30.7 | 20 | | 4.6 | | 0.6 | | 1 |
| Pregnancyan 35- | 24 | 2 | 8.3 | | | | | 3.9 | | 2 | | 1.25 |
| 36/40 | | | | | | | | | | | | |
| Pregnancyan 37- | 33 | 0 | 0.0 | 12.7 | 30.8 | | | 4 | | 1 | | 1 |
| 39/40 | | | | | | | | | | | | |
| Pregnancyan 40+ | 7 | 0 | 0.0 | — | — | | | 4.8 | | 1.5 | | 1.6 |
| Pregnancyan 40 n bp | 23 | 0 | 0.0 | 12.2 | 29.1 | 19 | | | | | | |
| Pregnancyan 40-42/40 + | 30 | | 0.0 | | | | | | | | | |
| BP | | | | | | | | | | | | |
| Pulmonary embolus | 12 | 1 | 8.3 | 11.2 | 28.2 | | | 4.6 | | 0 | | 0.9 |
| not on warf | | | | | | | | | | | | |
| Pulmonary embolus | 4 | 1 | 25.0 | | | | | | | | | |
| on warfarin | | | | | | | | | | | | |
| Pulmonary fibrosis | 4 | 0 | 0.0 | 9.1 | 30.1 | | | | | | | |
| Pulmonary | 5 | 0 | 0.0 | 11.7 | 31.7 | | | 4 | | 1 | | 2 |
| hypetension | | | | | | | | | | | | |
| Pyloric stenosis | 5 | 1 | 20.0 | 14.3 | 29.1 | | | 4.3 | | 1 | | 1.7 |
| Pyrexia of unknown | 16 | 6 | 37.5 | 11.4 | 30.1 | 16 | | 4.6 | | 0 | | 1.2 |
| origin | | | | | | | | | | | | |
| Quadriplegia | 5 | 0 | 0.0 | 11.4 | 31.6 | | | 4 | | 1 | | 0 |
| Reiters | 2 | 1 | 50.0 | 11.8 | 28.1 | 24 | — | 4 | | 1.5 | | 1.5 |
| Renal failure chronic | 275 | 135 | 49.1 | 10.2 | 28.7 | | | | | 5 | | |
| Renal failure: acute | 9 | 2 | | 10 | 27.8 | 21 | | 3.8 | | 2.7 | | 0.8 |
| Renal stone | 13 | 3 | 23.1 | | | | | 5 | | 1 | | 1.75 |
| Renal transplant | 19 | 9 | 47.4 | 12 | 28.7 | | | 4 | | 0.9 | | 1.6 |
| Respiratory distress | 4 | 1 | 25.0 | 10 | 31.4 | | | 3.7 | | 0.75 | | 0.8 |
| syndrome | | | | | | | | | | | | |
| Respiratory failure | 5 | 4 | 80.0 | | | | | | | | | |
| Rheumatoid arthritis | 18 | 1 | 5.6 | 10.3 | 27.8 | 26 | | 4 | | 0.2 | | 0.5 |
| pen | | | | | | | | | | | | |
| Rheumatoid arthritis | 63 | 5 | 7.9 | 12.4 | 26.9 | 23 | | | | | | |
| all | | | | | | | | | | | | |
| Rheumatoid arthritis | 9 | 2 | 22.2 | | | | | | | | | |
| au | | | | | | | | | | | | |
| Rheumatoid arthritis | 3 | 0 | 0.0 | 14 | 29.2 | 18 | | 4 | | 0.6 | | 1 |
| az | | | | | | | | | | | | |
| Rheumatoid arthritis | 3 | 0 | 0.0 | 9.6 | 30 | | | 5 | | | | |
| st | | | | | | | | | | | | |
| RT | 10 | 4 | 40.0 | 10.5 | 23.9 | 18 | | 4 | | 0.5 | | 1 |
| Sarcoid | 17 | 0 | 0.0 | 11.7 | 30.1 | 23 | | 3.5 | | 1 | | 1.5 |
| Sarcoma | 5 | 3 | 60.0 | 10.9 | 25.3 | 11 | | 4.7 | | 0 | | 1 |
| Satelitism | 1 | 0 | 0.0 | 12.7 | 30.2 | | | | | | | |
| Scleroderma | 6 | 0 | 0.0 | | | | | 4.5 | | 1.25 | | 1.8 |
| Scoliosis | 4 | 0 | 0.0 | 12.6 | 30.5 | | | 4.5 | | 0.2 | | 1.5 |
| Sepsis | 8 | 6 | 75.0 | 13.6 | 28.4 | | | | | | | |
| Sleep apnoea | 5 | 0 | 0.0 | | | | | | | | | |
| Spina bifida | 5 | 0 | 0.0 | | | | | | | | | |
| Splenectomy | 8 | 1 | 12.5 | | | 13 | | | | | | |
| Sprue | 1 | 1 | 100.0 | 11.3 | 27.8 | 23 | 4.8 | | | | | |
| Stem cells | 4 | 0 | 0.0 | | | | | | | | | |
| Subacute bacterial | 9 | 2 | 22.2 | | | | | 4 | | | | 1.5 |
| endocarditis | | | | | | | | | | | | |
| Syncope | 5 | 0 | 0.0 | | | | | | | | | |
| Systemic lupus | 7 | 0 | 0.0 | | | | | | | | | |
| erythethematosis | | | | | | | | | | | | |
| T's & As | 6 | 0 | 0.0 | 9.7 | 28 | 23 | | | | | | |
| Temperal arteritis | 4 | 3 | 75.0 | | | | | | | | | |
| Thalassemia beta trait | 81 | 1 | 1.2 | 9.7 | 28 | 26.4 | | 5.2 | | 0 | | 0.5 |
| Thrombocytopenia | 7 | 1 | 14.3 | 12.5 | 29.7 | 22 | | 4.2 | | 1 | | 1.5 |
| Thrombocytosis | 5 | 0 | 0.0 | 10.8 | 28.3 | | | 4.5 | | 1.3 | | 0.7 |
| Thrombotic | 2 | 0 | 0.0 | 12.1 | 32.6 | 20 | | 4 | | | | |
| thromb ocytopenic | | | | | | | | | | | | |
| purpura | | | | | | | | | | | | |
| Thyrotoxicosis | 7 | 0 | 0.0 | 12.8 | 28.4 | 24 | | 4.2 | | 0.5 | | 1.3 |
| Transient ischemic | 7 | 3 | 42.9 | 14.4 | 29 | 22 | | 4.2 | | 0.5 | | 1.2 |
| attacks | | | | | | | | | | | | |
| Turp | 17 | 14 | 82.4 | 12.5 | 29.9 | 24.6 | | 4.3 | | 0.1 | | 1.2 |
| Ulcerative colitis | 35 | 4 | 11.4 | 12.6 | 27.9 | 19.5 | | 3.9 | | 1 | | 1 |
| Urinary retention | 8 | 5 | 62.5 | 13.3 | 32.3 | | | 3.6 | | 0.9 | | 1.3 |
| Uterine fibroids | 22 | 1 | 4.5 | 14.4 | 30.9 | | | 3.8 | | 0.9 | | 1.1 |
| Ventric tachy | 12 | 3 | 25.0 | 13 | 30.9 | | | 4 | | 0.6 | | 1.4 |
| Volvulus sigmoid | 3 | 1 | 33.3 | 17.8 | 28.7 | 18 | | | | | | |
| Waldenstrom's | 1 | 0 | 0.0 | 9 | 30 | | 4 | | | 1.5 | | 0.5 |
| macroglobulinemia | | | | | | | | | | | | |

### EQUIVALENTS

The embodiments of the disclosure described above are intended to be merely exemplary; numerous variations and modifications will be apparent to those skilled in the art. All such variations and modifications are intended to be within the scope of the present invention as defined in any appended claims.

1. A method of identifying a subject in need of diagnostic assessment or therapeutic intervention, the method comprising steps of:
determining one or more RBC membrane permeability parameters from a sample of the subject's blood;
comparing the determined parameter to a reference control parameter selected from the group consisting of a negative reference control parameter, a positive reference control parameter, or both; and
identifying the subject as in need of when the determined parameter is not comparable to the negative reference control parameter and/or is comparable to the positive reference control parameter.

2. The method of claim 1, wherein the one or more RBC membrane permeability parameters are selected from coefficient of permeability (Cp), Pk0, isotonic volume (IsoV), spherical volume (SphV), maximum % change in cell volume (Inc %), peak height of Cell Scan Plot at 10% below maximum (W10), Pxmax, Pxmin, Pymax, Pymin, Py ratio, sphericity index, scaled sphericity index, slope of Fluid Flux Curve (slope_{FFC}), δ dynes, fragmentation grade, Cell Scan shape, FFC shape, and CPP.

3. The method of claim 1 or claim 2, wherein the one or more RBC membrane permeability parameters comprise Cp.

4. The method of claim 3, wherein the subject is identified as in need of when the determined Cp has a value that is at least 10% different from the negative reference control parameter and/or within 10% of the positive reference control parameter.

5. The method of claim 3 or claim 4, wherein the subject is identified as in need of when the determined Cp is less than about 3.5 mL/m² or greater than about 4.3 mL/m².

6. The method of any one of claims 1-5, wherein the one or more RBC membrane permeability parameters comprise Pk0.

7. The method of claim 6, wherein the subject is identified as in need of when the determined Pk0 has a value that is at least 4% different from the negative reference control parameter and/or within 4% of the positive reference control parameter.

8. The method of claim 6 or claim 7, wherein the subject is identified as in need of when the determined Pk0 is less than about 143 mOsm/kg or greater than about 153 mOsm/kg.

9. The method of any one of claims 1-8, wherein the one or more RBC membrane permeability parameters comprise spherical volume (SphV).

10. The method of claim 9, wherein the subject is identified as in need of when the determined SphV is at least 7% different from the negative reference control parameter and/or within 7% of the positive reference control parameter.

11. The method of claim 9 or claim 10, wherein the subject is identified as in need of when the determined SphV is less than about 158 femtoliters or greater than about 180 femtoliters.

12. The method of any one of claims 1-11, wherein the one or more RBC membrane permeability parameters comprise isotonic volume (IsoV).

13. The method of claim 12, wherein the subject is identified as in need of when the determined IsoV is at least 5% different from the negative reference control parameter and/or within 5% of the positive reference control parameter.

14. The method of claim 12 or claim 13, wherein the subject is identified as in need of when the determined IsoV is less than about 87 femtoliters or greater than about 96 femtoliters.

15. The method of any one of claims 1-14, wherein the one or more RBC membrane permeability parameters comprise Inc %.

16. The method of claim 15, wherein the subject is identified as in need of when the determined Inc % is at least 9% different from the negative reference control parameter and/or within 9% of the positive reference control parameter.

17. The method of claim 15 or claim 16, wherein the subject is identified as in need of when the determined Inc % is less than about 77% or greater than about 93%.

18. The method of any one of claims 1-17, wherein the one or more RBC membrane permeability parameters comprise W10.

19. The method of claim 18, wherein the subject is identified as in need of when the determined W10 is at least 7% different from the negative reference control parameter and/or within 7% of the positive reference control parameter.

20. The method of claim 18 or claim 19, wherein the subject is identified as in need of when the determined W10 is less than about 17 mOsm/kg or greater than about 20 mOsm/kg.

21. The method of any one of claims 1-20, wherein the one or more RBC membrane permeability parameters comprise Pxmax.

22. The method of claim 21, wherein the subject is identified as in need of when the determined Pxmax is at least 3% different from the negative reference control parameter and/or within 3% of the positive reference control parameter.

23. The method of claim 21 or claim 22, wherein the subject is identified as in need of when the determined Pxmax is less than about 159 mOsm/kg or greater than about 170 mOsm/kg.

24. The method of any one of claims 1-23, wherein the one or more RBC membrane permeability parameters comprise Pxmin.

25. The method of claim 24, wherein the subject is identified as in need of when the determined Pxmin is at least 5% different from the negative reference control parameter and/or within 5% of the positive reference control parameter.

26. The method of claim 24 or claim 25, wherein the subject is identified as in need of when the determined Pxmin is less than about 124 mOsm/kg or greater than about 137 mOsm/kg.

27. The method of any one of claims 1-26, wherein the one or more RBC membrane permeability parameters comprise Pymax.

28. The method of claim 27, wherein the subject is identified as in need of when the determined Pymax is at least 8% different from the negative reference control parameter and/or within 8% of the positive reference control parameter.

29. The method of claim 27 or claim 28, wherein the subject is identified as in need of when the determined Pymax is less than about 12 (fL·10⁻¹)/mOsm/kg or greater than about 14 (fL·10⁻¹)/mOsm/kg.

30. The method of any one of claims 1-29, wherein the one or more RBC membrane permeability parameters comprise Pymin.

31. The method of claim 30, wherein the subject is identified as in need of when the determined Pymin is at least 13% different from the negative reference control parameter and/or within 13% of the positive reference control parameter.

32. The method of claim 30 or claim 31, wherein the subject is identified as in need of when the determined Pymin is less than about −17 (fL·10⁻¹)/mOsm/kg or greater than about −22 (fL·10⁻¹)/mOsm/kg.

33. The method of any one of claims 1-32, wherein the one or more RBC membrane permeability parameters comprise Py ratio.

34. The method of claim 33, wherein the subject is identified as in need of when the determined Py ratio is at least 14% different from the negative reference control parameter and/or within 14% of the positive reference control parameter.

35. The method of claim 33 or claim 34, wherein the subject is identified as in need of when the determined Py ratio is less than about 0.6 or greater than about 0.8.

36. The method of any one of claims 1-35, wherein the one or more RBC membrane permeability parameters comprise sphericity index (SI).

37. The method of claim 36, wherein the subject is identified as in need of when the SI is at least 3% different from the negative reference control parameter and/or within at least 3% of the positive reference control parameter.

38. The method of claim 36 or claim 37, wherein the subject is identified as in need of when the SI is less than about 1.52 or greater than about 1.62.

39. The method of any one of claims 1-38, wherein the one or more RBC membrane permeability parameters comprise scaled sphericity index (sSI).

40. The method of claim 39, wherein the subject is identified as in need of when the sSI is at least 3% different from the negative reference control parameter and/or within at least 3% of the positive reference control parameter.

41. The method of claim 39 or claim 40, wherein the subject is identified as in need of when the sSI is less than about 15.2 or greater than about 16.2.

42. The method of any one of claims 1-41, wherein the one or more RBC membrane permeability parameters comprise slope_{FFC}.

43. The method of claim 42, wherein the subject is identified as in need of when the determined slope_{FFC} is less than about −0.1 (fL·10⁻¹)/(mOsm/kg)² or greater than about 1.5 (fL·10⁻¹)/(mOsm/kg)².

44. The method of any one of claims 1-43, wherein the one or more RBC membrane permeability parameters comprise δ dynes.

45. The method of claim 44, wherein the subject is identified as in need of when the δ dynes is at least 9% different from the negative reference control parameter and/or within at least 9% of the positive reference control parameter.

46. The method of claim 44 or claim 45, wherein the subject is identified as in need of when the δ dynes is less than about 31 dynes or greater than about 38 dynes.

47. The method of any one of claims 1-46, wherein the one or more RBC membrane permeability parameters comprise one or more features of Cell Scan shape.

48. The method of claim 47, wherein the subject is identified as in need of when the determined Cell Scan shape is greater than 1 on the scale described in Example 3.

49. The method of claim 47 or claim 48, wherein the subject is identified as in need of when the determined Cell Scan shape is not comparable to Cell Scan Shape N of FIG. 5.

50. The method of any one of claims 47-49, wherein the subject is identified as in need of when the determined Cell Scan shape is comparable to Cell Scan Shape L, Cell Scan Shape P, Cell Scan Shape G, Cell Scan Shape MF, Cell Scan Shape T, Cell Scan Shape HS, or Cell Scan Shape C of FIG. 5.

51. The method of claim 50, wherein the subject is identified as in need of diagnostic assessment or therapeutic intervention for leukemia or lymphoma when the Cell Scan shape is comparable to Cell Scan Shape L.

52. The method of claim 50, wherein the subject is identified as in need of diagnostic assessment or therapeutic intervention for pancreatic or lung cancer when the Cell Scan shape is comparable to Cell Scan Shape P.

53. The method of claim 50, wherein the subject is identified as in need of diagnostic assessment or therapeutic intervention for gastrointestinal tract malignancies when the Cell Scan shape is comparable to Cell Scan Shape G.

54. The method of claim 50, wherein the subject is identified as in need of diagnostic assessment or therapeutic intervention for preleukemic stage myelodysplasia when the Cell Scan shape is comparable to Cell Scan Shape MF.

55. The method of claim 50, wherein the subject is identified as in need of diagnostic assessment or therapeutic intervention for beta thalassemia heterozygotes, hemoglobin S homozygotes, and/or hemoglobin C homozygotes when the Cell Scan shape is comparable to Cell Scan Shape T.

56. The method of claim 50, wherein the subject is identified as in need of diagnostic assessment or therapeutic intervention for hereditary spherocytosis or hemolytic anemias when the Cell Scan shape is comparable to Cell Scan Shape HS.

57. The method of claim 50, wherein the subject is identified as in need of diagnostic assessment or therapeutic intervention for liver disease or cirrhosis when the Cell Scan shape is comparable to Cell Scan Shape C.

58. The method of any one of claims 1-57, wherein the one or more RBC membrane permeability parameters comprise one or more features of FFC shape.

59. The method of claim 58, wherein the subject is identified as in need of when the determined Cell Scan shape is not comparable to FFC Shape N of FIG. 6A.

60. The method of claim 58 or claim 59, wherein the subject is identified as in need of when the determined FFC shape is comparable to FFC Shape L of FIG. 6B, FFC Shape P of FIG. 6C, FFC Shape G of FIG. 6D, or FFC Shape T of FIG. 6E.

61. The method of claim 60, wherein the subject is identified as in need of diagnostic assessment or therapeutic intervention for leukemia or lymphoma when the FFC shape is comparable to FFC Shape L.

62. The method of claim 60, wherein the subject is identified as in need of diagnostic assessment or therapeutic intervention for pancreatic or lung cancer when the FFC shape is comparable to FFC Shape P.

63. The method of claim 60, wherein the subject is identified as in need of diagnostic assessment or therapeutic intervention for gastrointestinal tract malignancies when the FFC shape is comparable to FFC Shape G.

64. The method of claim 60, wherein the subject is identified as in need of diagnostic assessment or therapeutic intervention for beta thalassemia heterozygotes, hemoglobin S homozygotes, and/or hemoglobin C homozygotes when the FFC shape is comparable to FFC Shape T.

65. The method of any one of claims 1-64, wherein the one or more RBC membrane permeability parameters comprise fragmentation grade.

66. The method of claim 65, wherein the subject is identified as in need of when the determined fragmentation grade is greater than 2 on the scale described in Example 10.

67. The method of any one of claims 1-66, wherein the one or more RBC membrane permeability parameters comprise CPP.

68. The method of claim 67, wherein the subject is identified as in need of when the CPP is at least 20% different from the negative reference control parameter and/or within at least 20% of the positive reference control parameter.

69. The method of claim 67 or claim 68, wherein the subject is identified as in need of when the CPP is less than about 6.5 or greater than about 15.

70. The method of any one of claims 1-69, wherein the reference control parameter is a positive reference control parameter.

71. The method of any one of claims 1-69, wherein the reference control parameter is a negative reference control parameter.

72. The method of claim 71, wherein the negative reference control parameter is an average value determined from a population of healthy subjects.

73. The method of any one of claims 1-72, wherein the subject is susceptible to a particular disease, disorder, or condition.

74. The method of claim 73, wherein the disease disorder, or condition is cancer.

75. The method of claim 74, wherein the cancer is pancreatic cancer.

76. The method of claim 74, wherein the cancer is lung cancer.

77. The method of claim 74, wherein the cancer is brain cancer.

78. The method of claim 74, wherein the cancer is selected from pancreatic cancer, endometrial cancer, lymphoma, colon cancer, gall bladder cancer, prostate cancer, bladder cancer, rectal cancer, stomach cancer, ileum carcinoid carcinoma, acute myeloid leukemia, and bronchial cancer.

79. The method of claim 73, wherein the disease, disorder, or condition is pregnancy.

80. The method of claim 73, wherein the disease, disorder, or condition is a hemoglobinopathy.

81. The method of claim 73, wherein the disease, disorder, or condition is selected from thrombotic microangiopathy (TMA), glomerulonephritis, renal graft rejection, vasculitis, malignant hypertension, metastatic carcinoma, cardiac anomalies, heart valve hemolysis from pathological or prosthetic valves, severe burns, March hemoglobinuria, and HELLP syndrome.

82. The method of claim 73, wherein the disease, disorder, or condition is selected from Table 7.

83. The method of any one of claims 1-82, further comprising determining one or more clinical variables of the subject.

84. The method of any one of claims 1-83, further comprising administering suitable therapy to the subject identified as in need of.

85. A method of treating a disease, disorder, or condition in a subject, the method comprising administering suitable therapy to the subject in need thereof, wherein the subject has been identified as in need of based on one or more RBC membrane permeability parameters determined from a sample of the subject's blood.

86. A computer system for determining a quantitative probability that a subject has a particular disease, disorder, or condition, the computer system (i) being adapted to receive input relating to one or more RBC membrane permeability parameters determined from a sample of the subject's blood; (ii) optionally being further adapted to receive input relating to other clinical variables; (iii) comprising a processor for processing the received inputs by comparing them to a reference data set; and (iv) being adapted to display or transmit the quantitative probability.

87. A method of identifying RBC Permeability Modulating Agents, the method comprising steps of:
contacting a sample of blood from a healthy subject with an agent;
determining one or more RBC membrane permeability parameters from the sample of blood;
comparing the determined parameter to a reference control parameter selected from the group consisting of a positive reference control parameter, a negative reference control parameter, or both; and
identifying the agent as a RBC Permeability Modulating Agent when the determined parameter is not comparable to the negative reference control parameter and/or is comparable to the positive reference control parameter.

88. A method comprising steps of:
determining one or more RBC membrane permeability parameters from a sample of RBCs;
comparing the determined parameter to a reference control parameter selected from the group consisting of a positive reference control parameter, a negative reference control parameter, or both; and
identifying the sample of RBCs as not viable when the determined parameter is not comparable to the negative reference control parameter and/or is comparable to the positive reference control parameter.

89. The method of claim 88, wherein the sample of RBCs is a sample of blood.

90. The method of claim 88 or claim 89, wherein the sample of RBCs has been stored.

91. The method of any one of claims 88-90, wherein the sample of RBCs has been stored for greater than 6 weeks.

92. A method comprising steps of:
determining one or more RBC membrane permeability parameters from each of a plurality of blood samples obtained at different time points from a single subject; and
comparing the determined one or more parameters from a first time point with that from at least one later time point;
wherein a significant change in the determined one or more parameters over time indicates a material change in the subject's health state.

93. The method of claim 92, wherein the different time points are separated from one another by a reasonably consistent interval.

94. The method of claim 92 or claim 93, wherein the one or more RBC membrane permeability parameters are selected from coefficient of permeability (Cp), Pk0, isotonic volume (IsoV), spherical volume (SphV), maximum % change in cell volume (Inc %), peak height of Cell Scan Plot at 10% below maximum (W10), Pxmax, Pxmin, Pymax, Pymin, Py ratio, sphericity index, scaled sphericity index, slope of Fluid Flux Curve (slope_{FFC}), δ dynes, fragmentation grade, Cell Scan shape, FFC shape, and CPP.

95. The method of any one of claims 92-94, wherein a significant change is a change of 5% or greater in at least one of the determined one or more parameters.

96. The method of any one of claims 92-95, further comprising determining one or more clinical variables of the subject if a significant change in the determined one or more parameters over time is observed.

97. The method of any one of claims 92-96, further comprising administering suitable therapy to the subject if a significant change in the determined one or more parameters over time is observed.

98. A method comprising steps of:
determining one or more RBC membrane permeability parameters from a blood sample obtained from a subject for whom the one or more RBC membrane permeability parameters has previously been obtained at least once; and
comparing the determined one or more parameters with the previously obtained one or more parameters,
wherein a significant change in the determined one or more parameters compared to the previously obtained one or more parameters indicates a material change in the subject's health state.

99. The method of claim 98, wherein the one or more RBC membrane permeability parameters had previously been obtained for the subject at two or more distinct time points.

100. The method of claim 98 or claim 99, wherein the one or more RBC membrane permeability parameters are selected from coefficient of permeability (Cp), Pk0, isotonic volume (IsoV), spherical volume (SphV), maximum % change in cell volume (Inc %), peak height of Cell Scan Plot at 10% below maximum (W10), Pxmax, Pxmin, Pymax, Pymin, Py ratio, sphericity index, scaled sphericity index, slope of Fluid Flux Curve (slope_{FFC}), δ dynes, fragmentation grade, Cell Scan shape, FFC shape, and CPP.

101. The method of any one of claims 98-100, wherein a significant change is a change of 5% or greater in at least one of the determined one or more parameters.

102. The method of any one of claims 98-101, further comprising determining one or more clinical variables of the subject if a significant change in the determined one or more parameters compared to the previously obtained one or more parameters is observed.

103. The method of any one of claims 98-102, further comprising administering suitable therapy to the subject if a significant change in the determined one or more parameters compared to the previously obtained one or more parameters is observed.

104. A method comprising steps of:
determining one or more RBC membrane permeability parameters from a sample of a subject's blood;
comparing the determined parameter to a reference control parameter selected from the group consisting of a positive reference control parameter, a negative reference control parameter, or both; and
identifying a subject as likely to die within a time period when the determined parameter is not comparable to the negative reference control parameter and/or is comparable to the positive reference control parameter.

105. The method of claim 104, wherein the one or more RBC membrane permeability parameters are selected from are selected from coefficient of permeability (Cp), Pk0, isotonic volume (IsoV), spherical volume (SphV), maximum % change in cell volume (Inc %), peak height of Cell Scan Plot at 10% below maximum (W10), Pxmax, Pxmin, Pymax, Pymin, Py ratio, sphericity index, scaled sphericity index, slope of Fluid Flux Curve (slope_{FFC}), δ dynes, fragmentation grade, Cell Scan shape, FFC shape, and CPP.

106. The method of claim 105, wherein the one or more RBC membrane permeability parameters comprise Pk0.

107. The method of any one of claims 104-106, wherein the subject is identified as likely to die within a time period when the determined Pk0 has a value that is at least 5% different from the negative reference control parameter and/or within 5% of the positive reference control parameter.

108. The method of any one of claims 104-107, wherein the subject is identified as likely to die within about 70 months when the determined Pk0 is less than about 110 mOsm/kg or greater than about 170 mOsm/kg.

108. The method of any one of claims 104-106, wherein the subject is identified as likely to die within about 90 months when the determined Pk0 is less than about 125 mOsm/kg or greater than about 155 mOsm/kg.
